(19) United States
(12) Patent Application Publication
(10) Pub. No.: US 2022/0325301 A1
(43) Pub. Date: Oct. 13, 2022

(54) AUXOTROPHIC SELECTION METHODS

(71) Applicants: AUXOLYTIC LTD, London (GB); THE BOARD OF TRUSTEES OF THE LELAND STANFORD JUNIOR UNIVERSITY, Stanford, CA (US)

(72) Inventors: James Patterson, London, England (GB); Matthew Porteus, Stanford, CA (US); Volker Wiebking, Stanford, CA (US)

(73) Assignees: AUXOLYTIC LTD, London (GB); THE BOARD OF TRUSTEES OF THE LELAND STANFORD JUNIOR UNIVERSITY, Stanford, CA (US)

(21) Appl. No.: 17/608,741

(22) PCT Filed: May 8, 2020
(86) PCT No.: PCT/US2020/032114

§ 371 Date: Nov. 3, 2021

Related U.S. Application Data
(60) Provisional application No. 62/844,930, filed on May 8, 2019.
(60) Provisional application No. 62/904,725, filed on Sep. 24, 2019.

(51) Int. Cl. C12N 15/90; C12N 5/0783; C12N 5/078; C12N 5/077; C12N 5/071; C12N 9/22

(52) U.S. Cl. CPC C12N 15/907; C12N 5/0637; C12N 5/0644; C12N 5/0657; C12N 5/0676; C12N 5/069; C12N 9/22; C12Y 204/0201; C12Y 401/01023; C12N 2500/40; C12N 2501/115; C12N 2502/13; C12N 2506/45; C12N 2510/00; C12N 2533/90

(57) ABSTRACT

The present disclosure provides methods and compositions for generating populations of auxotrophic cells and populations of differentiated cells and selecting populations of transfected cells using split auxotrophy.

### CROSS-REFERENCE TO RELATED APPLICATIONS

The present application claims priority to U.S. Provisional Patent Application Nos. 62/844,930 filed May 8, 2019, and 62/904,725 filed Sep. 24, 2019, each of which is incorporated herein by reference in its entirety.

### SEQUENCE LISTING

The present application is being filed along with a Sequence Listing in electronic format. The Sequence Listing file, entitled 1191573PCT_SEQLST.txt, was created on Apr. 27, 2020, and is 66,244 bytes in size. The information in electronic format of the Sequence Listing is incorporated herein by reference in its entirety.

### FIELD OF THE DISCLOSURE

The present disclosure relates to methods of generating populations of differentiated cells and selecting populations of transfected cells.

### BACKGROUND OF THE DISCLOSURE

Cell therapies have been shown to provide promising treatments. Yet, reintroduction of modified cells into a human host carries risks including immune reactions, malignant transformation, or overproduction or lack of control of transgenes.

Several approaches of genetic engineering enable the control over functions of human cells like cell signaling, proliferation or apoptosis (see, e.g., Bonifant, Challice L., et al. “Toxicity and management in CAR T-cell therapy.” Molecular Therapy-Oncolytics 3 (2016): 16011; Sockolosky, Jonathan T., et al. “Selective targeting of engineered T cells using orthogonal IL-2 cytokine-receptor complexes.” Science 359.6379 (2018): 1037-1042; and Tey, Siok-Keen. “Adoptive T-cell therapy: adverse events and safety switches.” Clinical & translational immunology 3.6 (2014): e17; each of which is hereby incorporated by reference in its entirety) and make it possible to control even severe side effects of cell therapies (Bonifant et al., 2016). Despite these advances, other applications have been prevented from gaining widespread application, e.g. the use of engineered pluripotent cells for regenerative medicine (see, Ben-David and Benvenisty, 2011, Nat. Rev. Cancer 11, 268-277; Lee et al., 2013, Nat. Med. 19, 998-1004; Porteus, M. (2011) Mol. Ther. 19, 439-441; each of which is hereby incorporated by reference in its entirety), due to the fact that control systems that rely on the introduction of a genetically encoded control mechanism into the cell have multiple limitations (Tey, 2014).

Two of the major problems that can arise are “leakiness,” i.e. low-level activity of the mechanism in the absence of its trigger (see, Ando et al. (2015) Stem Cell Reports 5, 597-608, which is hereby incorporated by reference in its entirety), and the lack of removal of the entire cell population upon activation of the mechanism (see, Garin et al. (2001) Blood 97, 122-129; Di Stasi et al. (2011) N Engl J Med 365, 1673-1683; Wu et al. (2014) N Engl J Med 365, 1673-1683; Yagyu et al. (2015) Mol. Ther. 23, 1475-1485; each of which is hereby incorporated by reference in its entirety), due to several escape mechanisms from external control. For example, the transgene that is introduced by viral transduction can be silenced from expression by the cell (see, Sulkowski et al. (2018) Switch. Int. J. Mol. Sci. 19, 197, which is hereby incorporated by reference in its entirety) or the cell can develop resistance towards the effector mechanism (See, Yagyu et al. (2015) Mol. Ther. 23, 1475-1485, which are hereby incorporated by reference in its entirety). Another concern is the mutation of the transgene in cell types with genetic instability, e.g. cell lines that are cultured for prolonged periods of time or tumor cell lines (Merkle et al. (2017) Nature 545, 229-233; D'Antonio et al. (2018) Cell Rep. 24, 883-894; each of which is hereby incorporated by reference in its entirety). Moreover, primary cell populations often retain their functionality for only limited time in ex vivo culture and many types cannot be purified by clonal isolation.

Therefore, there is a long felt need, in the case of cell therapy products derived from proliferative progenitors, for a method of selection of differentiated non-proliferative cells in vitro, before their introduction into the patient. In general, production of differentiated cell products from multipotent progenitor cells has been hampered by an inability to select for the desired differentiated cell population of interest.

### SUMMARY OF THE DISCLOSURE

Various embodiments of the disclosure provide a method of generating a population of differentiated cells comprising: (a) contacting a plurality of progenitor cells with a CRISPR/Cas system comprising a guide RNA (gRNA) targeting an inessential portion of a promoter of a gene; (b) inserting biallelically by homologous recombination a construct comprising a tissue-specific promoter and at least a portion of the gene, wherein the gene is selected from the group consisting of: AACS, AADAT, AASDHPPT, AASS, ACAT1, ACCS, ACCSL, ACO1, ACO2, ACSS3, ADSL, ADSS, ADSSL1, ALAD, ALAS1, ALAS2, ALDH1A1, ALDH1A2, ALDH1A3, ALDH1B1, ALDH2, AMD1, ASL, ASS1, ATF4, ATF5, AZIN1, AZIN2, BCAT1, BCAT2, CAD, CBS, CBSL, CCBL1, CCBL2, CCS, CEBPA, CEBPB, CEBPD, CEBPE, CEBPG, CH25H, COQ6, CPS1, CTH, CYP51A1, DECR1, DHFR, DHFRL1, DHODH, DHRS7, DHRS7B, DHRS7C, DPYD, DUT, ETFDH, FAXDC2, FDFT1, FDPS, FDXR, FH, FPGS, G6PD, GCAT, GCH1, GCLC, GFPT1, GFPT2, GLRX5, GLUL, GMPS, GPT, GPT2, GSX2, H6PD, HAAO, HLCS, HMBS, HMGCL, HMGCLL1, HMGCS1, HMGCS2, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HRSP12, HSD11B1, HSD11B1L, HSD17B12, HSD17B3, HSD17B7, HSD17B7P2, HSDL1, HSDL2, IBA57, IDO1, IDO2, IL4I1, ILVBL, IP6K1, IP6K2, IP6K3, IPMK, IREB2, ISCA1, ISCA1P1, ISCA2, KATNA1, KATNAL1, KATNAL2, KDM1B, KDSR, KMO, KYNU, LGSN, LSS, MARS, MARS2, MAX, MITF, MLX, MMS19, MPC1, MPC1L, MPI, MSMO1, MTHFD1, MTHFD1L, MTHFD2, MTHFD2L, MTHFR, MTRR, MVK, MYB, MYBL1, MYBL2, NAGS, ODC1, OTC, PAICS, PAOX, PAPSS1, PAPSS2, PDHB, PDX1, PFAS, PIN1, PLCB1, PLCB2, PLCB3, PLCB4, PLCD1, PLCD3, PLCD4, PLCE1, PLCG1, PLCG2, PLCH1, PLCH2, PLCL1, PLCL2, PLCZ1, PM20D1, PPAT, PSAT1, PSPH, PYCR1, PYCR2, QPRT, RDH8, RPUSD2, SCD, SCD5, SLC25A19, SLC25A26, SLC25A34, SLC25A35, SLC7A10, SLC7A11, SLC7A13, SLC7A5, SLC7A6, SLC7A7, SLC7A8, SLC7A9, SMOX, SMS, SNAPC4, SOD1, SOD3, SQLE, SRM, TAT, TFE3, TFEB, TFEC, THNSL1, THNSL2, TKT, TKTL1, TKTL2, UMPS, UROD, UROS, USF1, USF2, VPS33A, VPS33B, VPS36, VPS4A, and VPS4B, resulting in the progenitor cells being auxotrophic for an auxotrophic factor; (c) contacting the plurality of progenitor cells with the auxotrophic factor; (d) stimulating differentiation of the progenitor cells into a tissue associated with the tissue-specific promoter, wherein the gene is expressed in response to differentiation; and (e) removing the auxotrophic factor, thereby selecting for differentiated cells to generate the population of differentiated cells. The portion of the promoter is inessential to ensure a simple IN/DEL in the region does not cause auxotrophy. Insertion of the tissue-specific promoter by homologous recombination will result in loss of nutrient-synthesizing gene expression in progenitor cells and thus auxotrophy. On differentiation, nutrient synthesizing gene expression will be switched on, and the nutrient can be removed with only differentiated cells surviving.

In certain embodiments, the method further comprises contacting the plurality of cells with 5-FOA. In certain embodiments, the gene is a UMPS gene. In certain embodiments, the tissue-specific promoter replaces the promoter for the UMPS gene. In certain embodiments, the auxotrophic factor is a source of uracil, e.g., uridine. In certain embodiments, the construct further comprises a nucleotide sequence encoding a therapeutic protein or therapeutic factor which is expressed in response to differentiation. In certain embodiments, the method further comprises expressing the therapeutic protein as a cassette with the at least a portion of the UMPS gene. In certain embodiments, the construct is polycistronic. In certain embodiments, the construct comprises an internal ribosome entry site (IRES) or a peptide 2A sequence (P2A). In certain embodiments, the at least a portion of the UMPS gene is a homology arm. In certain embodiments, the plurality of progenitor cells is selected from the group consisting of: hematopoietic stem cells (HSCs), embryonic stem cells, transdifferentiated stem cells, neural progenitor cells, mesenchymal stem cells, osteoblasts, and cardiomyocytes. In certain embodiments, the tissue is selected from the group consisting of: adipose tissue, adrenal gland, bladder, blood, bone, bone marrow, brain, cervix, connective tissue, ear, embryonic tissue, esophagus, eye, heart, hematopoietic tissue, intestine, kidney, larynx, liver, lung, lymph, lymph node, mammary gland, mouth, muscle, nerve, ovary, pancreas, parathyroid, pharynx, pituitary gland, placenta, prostate, salivary gland, skin, spleen, stomach, testis, thymus, thyroid, tonsil, trachea, umbilical cord, uterus, endocrine, neuronal tissue, and vascular tissue. In certain embodiments, the population of differentiated cells comprises immune cells. In certain embodiments, the immune cell is a T cell, a B cell, or a natural killer (NK) cell.

In certain embodiments, the tissue-specific promoter is selected from the group consisting of: WAS proximal promoter; CD4 mini-promoter/enhancer; CD2 locus control region; CD4 minimal promoter and proximal enhancer and silencer; CD4 mini-promoter/enhancer; GATA-1 enhancer HS2 within the LTR; Ankyrin-1 and α-spectrin promoters combined or not with HS-40, GATA-1, ARE and intron 8 enhancers; Ankyrin-1 promoter/β-globin HS-40 enhancer; GATA-1 enhancer HS1 to HS2 within the retroviral LTR; Hybrid cytomegalovirus (CMV) enhancer/β-actin promoter; MCH II-specific HLA-DR promoter; Fascin promoter (pFascin); Dectin-2 gene promoter; 5′ untranslated region from the DC-STAMP; Heavy chain intronic enhancer (Eμ) and matrix attachment regions; CD19 promoter; Hybrid immunoglobulin promoter (Igk promoter, intronic Enhancer and 3′ enhancer from Ig genes); CD68L promoter and first intron; Glycoprotein Ibα promoter; Apolipoprotein E (Apo E) enhancer/alpha1-antitrypsin (hAAT) promoter (ApoE/hAAT); HAAT promoter/Apo E locus control region; Albumin promoter; HAAT promoter/four copies of the Apo E enhancer; Albumin and hAAT promoters/al-microglobulin and prothrombin enhancers; HAAT promoter/Apo E locus control region; hAAT promoter/four copies of the Apo E enhancer; TBG promoter (thyroid hormone-binding globulin promoter and α1-microglobulin/bikunin enhancer); DC172 promoter (α1-antitrypsin promoter and α1-microglobulin enhancer); LCAT, kLSP-IVS, ApoE/hAAT and liver-fatty acid-binding protein promoters; RU486-responsive promoter; Creatine kinase promoter; Creatine kinase promoter; Synthetic muscle-specific promoter C5-12; Creatine kinase promoter; Hybrid enhancer/promoter regions of α-myosin and creatine kinase (MHCK7); Hybrid enhancer/promoter regions of α-myosin and creatine kinase; Synthetic muscle-specific promoter C5-12; Cardiac troponin-1 proximal promoter; E-selectin and KDR promoters; Prepro-endothelin-1 promoter; KDR promoter/hypoxia-responsive element; Flt-1 promoter; Flt-1 promoter; ICAM-2 promoter; Synthetic endothelial promoter; Endothelin-1 gene promoter; Amylase promoter; Insulin and human pdx-1 promoters; TRE-regulated insulin promoter; Enolase promoter; Enolase promoter; TRE-regulated synapsin promoter; Synapsin 1 promoter; PDGF-β promoter/CMV enhancer; PDGF-β, synapsin, tubulin-α and Ca2+/calmodulin-PK2 promoters combined with CMV enhancer; Phosphate-activated glutaminase and vesicular glutamate transporter-1 promoters; Glutamic acid decarboxylase-67 promoter; Tyrosine hydroxylase promoter; Neurofilament heavy gene promoter; Human red opsin promoter; Keratin-18 promoter; keratin-14 (K14) promoter; and Keratin-5 promoter. In certain embodiments of the method described herein, the construct is tagged with a conditional destabilization domain or a conditional ribozyme switch.

Various embodiments of the disclosure provide a method of generating a population of differentiated cells comprising: (a) contacting a plurality of progenitor cells with a DNA sequence encoding one or more progenitor cell-specific miRNA target sites, wherein the DNA sequence is knocked into an auxotrophy-inducing gene resulting in the progenitor cells being auxotrophic for an auxotrophic factor, and wherein a progenitor cell-specific miRNA that binds the miRNA target sites is expressed in the progenitor cells; (b) contacting the plurality of progenitor cells with the auxotrophic factor; (c) stimulating differentiation of the progenitor cells, wherein differentiation suppresses expression of the progenitor cell-specific miRNA and activates expression of the gene; and (d) removing the auxotrophic factor, thereby selecting for differentiated cells to generate a population of differentiated cells.

In certain embodiments, the method further comprises contacting the plurality of cells with 5-FOA. In certain embodiments, the auxotrophy-inducing gene is selected from the group consisting of: AACS, AADAT, AASDHPPT, AASS, ACAT1, ACCS, ACCSL, ACO1, ACO2, ACSS3, ADSL, ADSS, ADSSL1, ALAD, ALAS1, ALAS2, ALDH1A1, ALDH1A2, ALDH1A3, ALDH1B1, ALDH2, AMD1, ASL, ASS1, ATF4, ATF5, AZIN1, AZIN2, BCAT1, BCAT2, CAD, CBS, CBSL, CCBL1, CCBL2, CCS, CEBPA, CEBPB, CEBPD, CEBPE, CEBPG, CH25H, COQ6, CPS1, CTH, CYP51A1, DECR1, DHFR, DHFRL1, DHODH, DHRS7, DHRS7B, DHRS7C, DPYD, DUT, ETFDH, FAXDC2, FDFT1, FDPS, FDXR, FH, FPGS, G6PD, GCAT, GCH1, GCLC, GFPT1, GFPT2, GLRX5, GLUL, GMPS, GPT, GPT2, GSX2, H6PD, MAO, HLCS, HMBS, HMGCL, HMGCLL1, HMGCS1, HMGCS2, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HRSP12, HSD11B1, HSD11B1L, HSD17B12, HSD17B3, HSD17B7, HSD17B7P2, HSDL1, HSDL2, IBA57, IDO1, IDO2, IL4I1, ILVBL, IP6K1, IP6K2, IP6K3, IPMK, IREB2, ISCA1, ISCA1P1, ISCA2, KATNA1, KATNAL1, KATNAL2, KDM1B, KDSR, KMO, KYNU, LGSN, LSS, MARS, MARS2, MAX, MITF, MLX, MMS19, MPC1, MPC1L, MPI, MSMO1, MTHFD1, MTHFD1L, MTHFD2, MTHFD2L, MTHFR, MTRR, MVK, MYB, MYBL1, MYBL2, NAGS, ODC1, OTC, PAICS, PAOX, PAPSS1, PAPSS2, PDHB, PDX1, PFAS, PIN1, PLCB1, PLCB2, PLCB3, PLCB4, PLCD1, PLCD3, PLCD4, PLCE1, PLCG1, PLCG2, PLCH1, PLCH2, PLCL1, PLCL2, PLCZ1, PM20D1, PPAT, PSAT1, PSPH, PYCR1, PYCR2, QPRT, RDH8, RPUSD2, SCD, SCD5, SLC25A19, SLC25A26, SLC25A34, SLC25A35, SLC7A10, SLC7A11, SLC7A13, SLC7A5, SLC7A6, SLC7A7, SLC7A8, SLC7A9, SMOX, SMS, SNAPC4, SOD1, SOD3, SQLE, SRM, TAT, TFE3, TFEB, TFEC, THNSL1, THNSL2, TKT, TKTL1, TKTL2, UMPS, UROD, UROS, USF1, USF2, VPS33A, VPS33B, VPS36, VPS4A, and VPS4B.

In certain embodiments, the auxotrophy-inducing gene is uridine monophosphate synthetase (UMPS) and the one or more progenitor cell-specific miRNA target sites is present in an mRNA transcript transcribed from the UMPS gene. In certain embodiments, the one or more progenitor cell-specific miRNA target sites is in the 3′ untranslated region (UTR) of an mRNA transcript transcribed from the auxotrophy-inducing gene. In certain embodiments, the auxotrophic factor is a source of uracil, e.g., uridine. In certain embodiments, the method further comprises inserting into the genome of the progenitor cells a construct comprising a gene encoding a therapeutic factor, wherein expression of the therapeutic factor is controlled by the same promoter as the promoter controlling expression of the auxotrophy-inducing gene and the differentiated cells express the therapeutic factor. In certain embodiments, the method further comprises expressing the therapeutic protein as a cassette in-frame with the auxotrophy-inducing gene. In certain embodiments, the cassette comprises an internal ribosome entry site (IRES) or a peptide 2A sequence (P2A). In certain embodiments, the DNA sequence encoding the one or more progenitor cell-specific miRNA target sites further comprises a homology arm targeting the auxotrophy-inducing gene. In certain embodiments, the plurality of progenitor cells is selected from the group consisting of: hematopoietic stem cells (HSCs), embryonic stem cells, transdifferentiated stem cells, neural progenitor cells, mesenchymal stem cells, osteoblasts, and cardiomyocytes, and combinations thereof. In certain embodiments, the stimulating differentiation of the progenitor cells produces differentiated cells of a cell or tissue type selected from the group consisting of: adipose tissue, adrenal gland, ascites, bladder, blood, bone, bone marrow, brain, cervix, connective tissue, ear, embryonic tissue, esophagus, eye, heart, hematopoietic tissue, intestine, kidney, larynx, liver, lung, lymph, lymph node, mammary gland, mouth, muscle, nerve, ovary, pancreas, parathyroid, pharynx, pituitary gland, placenta, prostate, salivary gland, skin, spleen, stomach, testis, thymus, thyroid, tonsil, trachea, umbilical cord, uterus, endocrine, neuronal tissue, and vascular tissue. In certain embodiments, the population of differentiated cells comprises immune cells. For example, the immune cells can be selected from the group consisting of T cells, B cells, natural killer (NK) cells, and combinations thereof.

Various embodiments of the disclosure provide a method of treating a disease, disorder, or condition in a subject, the method comprising: administering to the subject a purified population of the differentiated cells.

Various embodiments of the disclosure provide a method of alleviating auxotrophy by producing an auxotrophic factor upon differentiation, the method comprising: (a) providing a plurality of auxotrophic progenitor cells, which have been generated by knockout of an auxotrophy-inducing gene; and (b) inserting a construct comprising an open reading frame of the auxotrophy-inducing gene into a tissue-specific gene locus, wherein expression of the tissue-specific gene is not disrupted, thereby producing the auxotrophic factor upon differentiation of the progenitor cells into the tissue associated with the tissue-specific gene locus.

In certain embodiments, the progenitor cells are selected from induced pluripotent stem cells (iPSCs) or embryonic stem cells (ESCs). In certain embodiments, the gene is selected from the group consisting of: a gene selected from the group consisting of: AACS, AADAT, AASDHPPT, AASS, ACAT1, ACCS, ACCSL, ACO1, ACO2, ACSS3, ADSL, ADSS, ADSSL1, ALAD, ALAS1, ALAS2, ALDH1A1, ALDH1A2, ALDH1A3, ALDH1B1, ALDH2,AMD1, ASL, ASS1, ATF4, ATF5, AZIN1, AZIN2, BCAT1, BCAT2, CAD, CBS, CBSL, CCBL1, CCBL2, CCS, CEBPA, CEBPB, CEBPD, CEBPE, CEBPG, CH25H, COQ6, CPS1, CTH, CYP51A1, DECR1, DHFR, DHFRL1, DHODH, DHRS7, DHRS7B, DHRS7C, DPYD, DUT, ETFDH, FAXDC2, FDFT1, FDPS, FDXR, FH, FPGS, G6PD, GCAT, GCH1, GCLC, GFPT1, GFPT2, GLRX5, GLUL, GMPS, GPT, GPT2, GSX2, H6PD, MAO, HLCS, HMBS, HMGCL, HMGCLL1, HMGCS1, HMGCS2, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HRSP12, HSD11B1, HSD11B1L, HSD17B12, HSD17B3, HSD17B7, HSD17B7P2, HSDL1, HSDL2, IBA57, IDO1, IDO2, IL4I1, ILVBL, IP6K1, IP6K2, IP6K3, IPMK, IREB2, ISCA1, ISCA1P1, ISCA2, KATNA1, KATNAL1, KATNAL2, KDM1B, KDSR, KMO, KYNU, LGSN, LSS, MARS, MARS2, MAX, MITF, MLX, MMS19, MPC1, MPC1L, MPI, MSMO1, MTHFD1, MTHFD1L, MTHFD2, MTHFD2L, MTHFR, MTRR, MVK, MYB, MYBL1, MYBL2, NAGS, ODC1, OTC, PAICS, PAOX, PAPSS1, PAPSS2, PDHB, PDX1, PFAS, PIN1, PLCB1, PLCB2, PLCB3, PLCB4, PLCD1, PLCD3, PLCD4, PLCE1, PLCG1, PLCG2, PLCH1, PLCH2, PLCL1, PLCL2, PLCZ1, PM20D1, PPAT, PSAT1, PSPH, PYCR1, PYCR2, QPRT, RDH8, RPUSD2, SCD, SCD5, SLC25A19, SLC25A26, SLC25A34, SLC25A35, SLC7A10, SLC7A11, SLC7A13, SLC7A5, SLC7A6, SLC7A7, SLC7A8, SLC7A9, SMOX, SMS, SNAPC4, SOD1, SOD3, SQLE, SRM, TAT, TFE3, TFEB, TFEC, THNSL1, THNSL2, TKT, TKTL1, TKTL2, UMPS, UROD, UROS, USF1, USF2, VPS33A, VPS33B, VPS36, VPS4A, and VPS4B. In certain embodiments, the gene is uridine monophosphate synthase (UMPS). In certain embodiments, the construct further comprises an internal ribosome entry site (IRES) or a peptide 2A sequence (P2A). In certain embodiments, the tissue-specific gene locus is an insulin locus. Certain embodiments further comprise differentiating the plurality of auxotrophic progenitor cells to immune cells. The immune cells can comprise T cells, B cells, or natural killer (NK) cells. In certain embodiments, the tissue-specific genes are not replaced during the inserting step. In certain embodiments, the method further comprises producing insulin upon differentiation of the progenitor cells. In certain embodiments of the method described herein, the gene is tagged with a conditional destabilization domain or a conditional ribozyme switch.

Various embodiments of the disclosure provide a method of selecting cells with plasmid integration or episomal expression, i.e., having functionally integrated at least an exogenous gene, the method comprising: (a) providing a plurality of cells with a knockout of an auxotrophy-inducing gene resulting in auxotrophy, i.e., resulting in a plurality of cells requiring the auxotrophic factor, wherein the plurality of cells is grown in a medium providing the auxotrophic factor to the plurality of cells; (b) transfecting the plurality of cells with a delivery system selected from the group consisting of a plasmid, a lentivirus, an adeno-associated virus (AAV), and a nanoparticle, wherein the delivery system expresses the auxotrophic factor; and (c) removing the medium, thereby selecting cells with plasmid integration or episomal expression, i.e., cells having functionally integrated the exogenous gene.

In certain embodiments, the delivery system expresses at least one transgene. In certain embodiments, the gene is selected from the group consisting of: AACS, AADAT, AASDHPPT, AASS, ACAT1, ACCS, ACCSL, ACO1, ACO2, ACSS3, ADSL, ADSS, ADSSL1, ALAD, ALAS1, ALAS2, ALDH1A 1, ALDH1A2, ALDH1A3, ALDH1B1, ALDH2, AMD1, ASL, ASS1, ATF4, ATF5, AZIN1, AZIN2, BCAT1, BCAT2, CAD, CBS, CBSL, CCBL1, CCBL2, CCS, CEBPA, CEBPB, CEBPD, CEBPE, CEBPG, CH25H, COQ6, CPS1, CTH, CYP51A1, DECR1, DHFR, DHFRL1, DHODH, DHRS7, DHRS7B, DHRS7C, DPYD, DUT, ETFDH, FAXDC2, FDFT1, FDPS, FDXR, FH, FPGS, G6PD, GCAT, GCH1, GCLC, GFPT1, GFPT2, GLRX5, GLUL, GMPS, GPT, GPT2, GSX2, H6PD, HAAO, HLCS, HMBS, HMGCL, HMGCLL1, HMGCS1, HMGCS2, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HRSP12, HSD11B1, HSD11B1L, HSD17B12, HSD17B3, HSD17B7, HSD17B7P2, HSDL1, HSDL2, IBA57, IDO1, IDO2, IL4I1, ILVBL, IP6K1, IP6K2, IP6K3, IPMK, IREB2, ISCA1, ISCA1P1, ISCA2, KATNA1, KATNAL1, KATNAL2, KDM1B, KDSR, KMO, KYNU, LGSN, LSS, MARS, MARS2, MAX, MITF, MLX, MMS19, MPC1, MPC1L, MPI, MSMO1, MTHFD1, MTHFD1L, MTHFD2, MTHFD2L, MTHFR, MTRR, MVK, MYB, MYBL1, MYBL2, NAGS, ODC1, OTC, PAICS, PAOX, PAPSS1, PAPSS2, PDHB, PDX1, PFAS, PIN1, PLCB1, PLCB2, PLCB3, PLCB4, PLCD1, PLCD3, PLCD4, PLCE1, PLCG1, PLCG2, PLCH1, PLCH2, PLCL1, PLCL2, PLCZ1, PM20D1, PPAT, PSAT1, PSPH, PYCR1, PYCR2, QPRT, RDH8, RPUSD2, SCD, SCD5, SLC25A19, SLC25A26, SLC25A34, SLC25A35, SLC7A10, SLC7A11, SLC7A13, SLC7A5, SLC7A6, SLC7A7, SLC7A8, SLC7A9, SMOX, SMS, SNAPC4, SOD1, SOD3, SQLE, SRM, TAT, TFE3, TFEB, TFEC, THNSL1, THNSL2, TKT, TKTL1, TKTL2, UMPS, UROD, UROS, USF1, USF2, VPS33A, VPS33B, VPS36, VPS4A, and VPS4B. In certain embodiments of the method described herein, the gene is tagged with a conditional destabilization domain or a conditional ribozyme switch.

Various embodiments of the disclosure provide a kit comprising the materials for performing the methods described herein.

In some embodiments, the methods described herein provide methods of generating a population of differentiated cells comprising contacting progenitor cells with a CRISPR/Cas system comprising a guide RNA (gRNA) targeting biallelically a portion of an auxotrophy-inducing gene. The biallelic targeting can knockout or knockdown the auxotrophy-inducing gene, for example, by interrupting the open reading frame or a regulatory sequence, or by introducing a target sequence for protein or nucleotide suppression or degradation. In embodiments where the auxotrophy-inducing gene comprises at least a first and a second independent functional domain, knockout or knockdown of the gene results in the progenitor cells being auxotrophic for each independent functional domain. Upon inducing auxotrophy in the progenitor cells, a first homologous recombination construct and a second homologous recombination construct can be introduced into the cells, the first homologous recombination construct comprising a first tissue-specific promoter and at least a portion of the first independent functional domain of the auxotrophy-inducing gene, and the second homologous recombination construct comprising a second tissue-specific promoter and at least a portion of the second independent functional domain of the auxotrophy-inducing gene. The progenitor cells can be grown in the presence of the auxotrophic factor and differentiation of the cells can be stimulated to produce differentiated cells (e.g., a cell type or tissue) expressing the first and the second tissue-specific promoters, resulting in the first and the second homologous recombination constructs being expressed in the differentiated cells. In this way, removing the auxotrophic factor eliminates cells lacking the first and the second independent functional domains and selects for cells having both domains functionally integrated.

In some embodiments, the auxotrophy-inducing gene has 2 or more independent functional domains, e.g., 3, 4, or 5 independent functional domains, or more than 5 independent functional domains, and re-expressing each independent functional domain in the auxotrophic cells is required to alleviate the auxotrophy, thereby enabling for selection of cells that express 2, 3, 4, 5, or more tissue-specific promoters by modifying the cells with 2, 3, 4, 5, or more homologous recombination constructs expressing the different independent functional domains under the regulation of different tissue-specific promoters expressed in the desired differentiated cell type or tissue.

In some embodiments, the auxotrophy-inducing gene is uridine monophosphate synthase (UMPS), the first independent functional domain comprises orotate phosphoribosyltransferase (OPRT), and the second independent functional domain comprises orotidine 5′-phosphate decarboxylase (ODC).

In some embodiments, the auxotrophy-inducing gene is carbamoyl-phosphate synthetase 2, aspartate transcarbamylase, and dihydroorotase (CAD), the first independent functional domain comprises carbamoyl-phosphate synthetase 2, the second independent functional domain comprises aspartate transcarbamylase, and the third independent functional domain comprises dihydroorotase.

In some embodiments, the methods further comprise contacting the cells with 5-FOA.

In some embodiments, one or more of the homologous recombination constructs are inserted into a safe harbor locus, e.g., CCR5.

In some embodiments, the auxotrophic factor is uridine.

In some embodiments, one or more of the homologous recombination constructs further comprise a nucleotide sequence encoding a therapeutic factor. One or more of the homologous recombination constructs can be polycistronic, e.g., with an internal ribosome entry site (IRES) or a peptide 2A sequence (P2A) separating, e.g., the coding sequence encoding the independent functional domain and the coding sequence encoding a therapeutic factor.

Exemplary progenitor cells for use in the methods described herein include, but are not limited to, hematopoietic stem cells (HSCs), embryonic stem cells, transdifferentiated stem cells, neural progenitor cells, mesenchymal stem cells, osteoblasts, and cardiomyocytes.

Examples of differentiated cell types or tissues for use in the methods described herein include, but are not limited to, adipose tissue, adrenal gland, ascites, bladder, blood, bone, bone marrow, brain, cervix, connective tissue, ear, embryonic tissue, esophagus, eye, heart, hematopoietic tissue, intestine, kidney, larynx, liver, lung, lymph, lymph node, mammary gland, mouth, muscle, nerve, ovary, pancreas, parathyroid, pharynx, pituitary gland, placenta, prostate, salivary gland, skin, spleen, stomach, testis, thymus, thyroid, tonsil, trachea, umbilical cord, uterus, endocrine, neuronal, and vascular.

In some embodiments, the differentiated cell is an immune cell, e.g., a T cell, a B cell, or a natural killer (NK) cell.

Examples of tissue-specific promoters for use in the methods described herein include, but are not limited to: WAS proximal promoter; CD4 mini-promoter/enhancer; CD2 locus control region; CD4 minimal promoter and proximal enhancer and silencer; CD4 mini-promoter/enhancer; GATA-1 enhancer HS2 within the LTR; Ankyrin-1 and α-spectrin promoters combined or not with HS-40, GATA-1, ARE and intron 8 enhancers; Ankyrin-1 promoter/β-globin HS-40 enhancer; GATA-1 enhancer HS1 to HS2 within the retroviral LTR; Hybrid cytomegalovirus (CMV) enhancer/β-actin promoter; MCH II-specific HLA-DR promoter; Fascin promoter (pFascin); Dectin-2 gene promoter; 5′ untranslated region from the DC-STAMP; Heavy chain intronic enhancer (Ep) and matrix attachment regions; CD19 promoter; Hybrid immunoglobulin promoter (Igk promoter, intronic Enhancer and 3′ enhancer from Ig genes); CD68L promoter and first intron; Glycoprotein Ibα promoter; Apolipoprotein E (Apo E) enhancer/alpha1-antitrypsin (hAAT) promoter (ApoE/hAAT); HAAT promoter/Apo E locus control region; Albumin promoter; HAAT promoter/four copies of the Apo E enhancer; Albumin and hAAT promoters/al-microglobulin and prothrombin enhancers; HAAT promoter/Apo E locus control region; hAAT promoter/four copies of the Apo E enhancer; TBG promoter (thyroid hormone-binding globulin promoter and α1-microglobulin/bikunin enhancer); DC172 promoter (α1-antitrypsin promoter and α1-microglobulin enhancer); LCAT, kLSP-IVS, ApoE/hAAT and liver-fatty acid-binding protein promoters; RU486-responsive promoter; Creatine kinase promoter; Creatine kinase promoter; Synthetic muscle-specific promoter C5-12; Creatine kinase promoter; Hybrid enhancer/promoter regions of α-myosin and creatine kinase (MHCK7); Hybrid enhancer/promoter regions of α-myosin and creatine kinase; Synthetic muscle-specific promoter C5-12; Cardiac troponin-1 proximal promoter; E-selectin and KDR promoters; Prepro-endothelin-1 promoter; KDR promoter/hypoxia-responsive element; Flt-1 promoter; Flt-1 promoter; ICAM-2 promoter; Synthetic endothelial promoter; Endothelin-1 gene promoter; Amylase promoter; Insulin and human pdx-1 promoters; TRE-regulated insulin promoter; Enolase promoter; Enolase promoter; TRE-regulated synapsin promoter; Synapsin 1 promoter; PDGF-β promoter/CMV enhancer; PDGF-β, synapsin, tubulin-α and Ca2+/calmodulin-PK2 promoters combined with CMV enhancer; Phosphate-activated glutaminase and vesicular glutamate transporter-1 promoters; Glutamic acid decarboxylase-67 promoter; Tyrosine hydroxylase promoter; Neurofilament heavy gene promoter; Human red opsin promoter; Keratin-18 promoter; keratin-14 (K14) promoter; and Keratin-5 promoter.

In some embodiments, one or more of the homologous recombination constructs further comprises a nucleotide sequence encoding a conditional destabilization domain or a conditional ribozyme switch. In this manner, the auxotrophy of the modified cells described herein can be further regulated by triggering a condition for destabilization of an independent functional domain or a condition for degradation of a message RNA encoding an independent functional domain. The condition can be, for example, the presence of a ligand that stabilizes the destabilization domain, or the absence of the ligand thereby inducing destabilization and degradation of the independent functional domain.

The differentiated population of cells generated using the methods described herein can be administered to a subject. In some embodiments, the differentiated cells are immune cells carrying a therapeutic factor and the subject is in need of or suspected to be in need of the therapeutic factor.

Also provided are methods of alleviating auxotrophy comprising providing a plurality of auxotrophic progenitor cells which have been generated by knockout or knockdown of an auxotrophy-inducing gene, wherein the gene comprises at least a first independent functional domain and a second independent functional domain, and inserting into the genome of the auxotrophic progenitor cells a first construct comprising an open reading frame of the first independent functional domain into a first tissue-specific gene locus, and inserting a second construct comprising an open reading frame of the second independent functional domain into a second tissue-specific gene locus. In some embodiments, expression of the tissue-specific genes at the first and second loci is not disrupted. Thus, auxotrophy is thereby alleviated upon differentiation of the progenitor cells into a cell type or tissue expressing the first and the second tissue-specific genes at the first and second loci.

Exploitation of auxotrophy-inducing genes comprising more than 2 independent functional domains is contemplated. For example, the auxotrophy-inducing genes can comprise, 2, 3, 4, 5, or more independent functional domains, such that re-expression of each of the 2, 3, 4, 5, or more independent functional domains is required to alleviate auxotrophy. Where the respective independent functional domains are inserted into the genome of the auxotrophic progenitor cells at respective tissue-specific gene loci, only cells expressing tissue-specific promoters corresponding to each of the first, second, third, fourth, and/or fifth tissue-specific loci having integrated respective independent functional domains will survive removal of the auxotrophic factor.

In some embodiments, the progenitor cells are induced pluripotent stem cells (iPSCs) or embryonic stem cells (ESCs).

In some embodiments, the auxotrophy-inducing gene is uridine monophosphate synthase (UMPS), the first independent functional domain comprises orotate phosphoribosyltransferase (OPRT), and the second independent functional domain comprises orotidine 5′-phosphate decarboxylase (ODC).

In some embodiments, the auxotrophy-inducing gene is carbamoyl-phosphate synthetase 2, aspartate transcarbamylase, and dihydroorotase (CAD), the first independent functional domain comprises carbamoyl-phosphate synthetase 2, the second independent functional domain comprises aspartate transcarbamylase, and the third independent functional domain comprises dihydroorotase.

In some embodiments, one or more of the constructs are polycistronic additionally encoding, for example, a therapeutic factor and further comprising an internal ribosome entry site (IRES) or a peptide 2A sequence (P2A) regulating expression of the cistrons of the construct(s).

In some embodiments, the tissue-specific gene locus is an insulin locus.

In some embodiments, the method further comprises differentiating the plurality of auxotrophic progenitor cells to immune cells, e.g., T cells, B cells, or natural killer (NK) cells.

In some embodiments, the tissue-specific genes are not replaced during the inserting step.

In some embodiments, differentiated cells produce insulin.

In some embodiments, one or more of the constructs comprise a nucleotide sequence encoding a conditional destabilization domain or a conditional ribozyme switch.

Also provided are methods of selecting cells having functionally integrated at least a first exogenous gene and a second exogenous gene. The methods can comprise providing a plurality of cells with a knockout or knockdown of an auxotrophy-inducing gene comprising at least a first and a second independent functional domain, resulting in auxotrophy for an auxotrophic factor in the plurality of cells. The cells can be grown in a medium providing the auxotrophic factor, and can be transfected with a first delivery system comprising a nucleotide sequence encoding the first exogenous gene and a nucleotide sequence encoding the first independent functional domain and a second delivery system comprising a nucleotide sequence encoding the second exogenous gene and a nucleotide sequence encoding the second independent functional domain. Upon replacement of the medium with a medium lacking the auxotrophic factor, cells that have not functionally integrated both the first and the second exogenous genes will remain auxotrophic and will not persist in culture, thereby selecting for cells that have functionally integrated the first and second exogenous genes.

The methods described further contemplate exploitation of auxotrophy-inducing genes having additional independent functional domains, e.g., auxotrophy-inducing genes having 2, 3, 4, 5, or more independent functional domains, such that re-expression of each of the independent functional domains is required to alleviate auxotrophy in the modified cells.

In some embodiments, the methods comprise transfecting the plurality of cells with a delivery system corresponding to each functional domain of the auxotrophy-inducing gene, wherein each delivery system comprises a nucleotide sequence encoding an exogenous gene and a nucleotide sequence encoding an independent functional domain. In some embodiments, one or more of the delivery systems comprises a plasmid, a lentivirus, an adeno-associated virus (AAV), or a nanoparticle.

In some embodiments, the auxotrophy-inducing gene is uridine monophosphate synthase (UMPS), the first independent functional domain comprises orotate phosphoribosyltransferase (OPRT), and the second independent functional domain comprises orotidine 5′-phosphate decarboxylase (ODC).

In some embodiments, the auxotrophy-inducing gene is carbamoyl-phosphate synthetase 2, aspartate transcarbamylase, and dihydroorotase (CAD), the first independent functional domain comprises carbamoyl-phosphate synthetase 2, the second independent functional domain comprises aspartate transcarbamylase, and the third independent functional domain comprises dihydroorotase.

Also provided are methods of generating a population of mature human beta cells comprising: (a) contacting a plurality of progenitor cells with a CRISPR/Cas system comprising a gRNA targeting biallelically a portion of a human UMPS gene resulting in the progenitor cells being auxotrophic for uridine; (b) contacting the plurality of progenitor cells with a first homologous recombination construct and a second homologous recombination construct, the first homologous recombination construct comprising a nucleotide sequence encoding insulin or an insulin-dependent expression control sequence operably linked to a first independent functional domain of UMPS, and the second homologous recombination construct comprising a nucleotide sequence encoding Nkx6.1 or an Nkx6.1-dependent expression control sequence operably linked to a second independent functional domain of UMPS, wherein the first and the second independent functional domains are selected from OPRT and ODC and are expressed only in progenitor cells expressing both insulin and Nkx6.1; (c) contacting the plurality of progenitor cells with uridine; (d) stimulating differentiation of the plurality of progenitor cells into mature beta cells; and (e) selecting for mature beta cells expressing both insulin and Nkx6.1 by removing uridine, thereby generating the population of mature human beta cells.

Also provided are methods of alleviating type 1 diabetes in a subject comprising: administering to the subject the mature human beta cells produced according to the methods described herein.

Also provided are mature human beta cells selected from a population of in vitro differentiated progenitor cells, the mature human beta cell comprising a biallelic genetic modification of an auxotrophy-inducing gene resulting in auxotrophy for an auxotrophic factor and one or more transgenes re-expressing the auxotrophy-inducing gene or one or more independent functional domains of the auxotrophy-inducing gene. The auxotrophy-inducing gene can be UMPS, the auxotrophic factor can be uridine, the independent functional domains can be selected from OPRT and ODC, and the one or more transgenes can further comprise a nucleotide sequence encoding insulin or an insulin-dependent expression control sequence and a nucleotide sequence encoding Nkx6.1 or an Nkx6.1-dependent expression control sequence.

Also provided are methods of generating a sub-population of human cardiomyocytes comprising: (a) contacting a plurality of progenitor cells with a CRISPR/Cas system comprising a gRNA targeting biallelically a portion of a human UMPS gene resulting in the progenitor cells being auxotrophic for uridine; (b) contacting the plurality of progenitor cells with a first homologous recombination construct and a second homologous recombination construct, the first homologous recombination construct comprising a nucleotide sequence encoding TBX5 or a TBX5-dependent expression control sequence operably linked to a first independent functional domain of UMPS, and the second homologous recombination construct comprising a nucleotide sequence encoding NKX2-5 or a NKX2-5-dependent expression control sequence operably linked to a second independent functional domain of UMPS, wherein the first and the second independent functional domains are selected from OPRT and ODC and are expressed only in progenitor cells expressing one or both of TBX5 and NKX2-5; (c) contacting the plurality of progenitor cells with uridine; (d) stimulating differentiation of the plurality of progenitor cells into cardiomyocytes; and (e) selecting for a sub-population of cardiomyocytes expressing one or both of TBX5 and NKX2-5 by removing uridine, thereby generating the sub-population of human cardiomyocytes.

In some embodiments, cells expressing both TBX5 and NKX2-5 represent a sub-population comprising ventricular cardiomyocytes.

In some embodiments, cells expressing TBX5 but not NKX2-5 represent a sub-population comprising nodal cardiomyocytes.

In some embodiments, cells not expressing TBX5 but expressing NKX2-5 represent a sub-population comprising atrial cardiomyocytes.

In some embodiments, cells expressing neither TBX5 nor NKX2-5 represent endothelial cells.

The disclosure further provides cardiomyocytes selected from a population of in vitro differentiated cardiomyocytes comprising a biallelic genetic modification of an auxotrophy-inducing gene resulting in auxotrophy for an auxotrophic factor and one or more transgenes re-expressing the auxotrophy-inducing gene or one or more independent functional domains of the auxotrophy-inducing gene. The auxotrophy-inducing gene can be UMPS, the auxotrophic factor can be uridine, the independent functional domains can be selected from OPRT and ODC, and the one or more transgenes can further comprise a nucleotide sequence encoding TBX5 or a TBX5-dependent expression control sequence and a nucleotide sequence encoding NKX2-5 or a NKX2-5-dependent expression control sequence.

In some embodiments, the cardiomyocyte belongs to a sub-population of cardiomyocytes selected from the group consisting of: first heart field lineage cells, ventricular cardiomyocytes, epicardial lineage cells, nodal cardiomyocytes, second heart field lineage cells, and atrial cardiomyocytes.

The disclosure also provides for use of the cardiomyocytes described herein in a method of in vitro drug testing.

Also provided are methods of generating a population of stable T reg cells comprising: (a) contacting a plurality of progenitor cells with a CRISPR/Cas system comprising a gRNA targeting biallelically a portion of a human UMPS gene resulting in the progenitor cells being auxotrophic for uridine; (b) contacting the plurality of progenitor cells with a first homologous recombination construct and a second homologous recombination construct, the first homologous recombination construct comprising a nucleotide sequence encoding FOXP3 or a FOXP3-dependent expression control sequence operably linked to a first independent functional domain of UMPS, and the second homologous recombination construct comprising a nucleotide sequence encoding a cell naïveté-associated promoter or an expression control sequence of a cell naïveté-associated promoter operably linked to a second independent functional domain of UMPS, wherein the first and the second independent functional domains are selected from OPRT and ODC and are expressed only in progenitor cells expressing both FOXP3 and a gene associated with the cell naïveté-associated promoter; (c) contacting the plurality of progenitor cells with uridine; (d) stimulating differentiation of the plurality of progenitor cells into stable T reg cells; and (e) selecting for stable T reg cells expressing both FOXP3 and the gene associated with the cell naïveté-associated promoter by removing uridine, thereby generating the population of stable T reg cells.

In some embodiments, the cell naïveté-associated promoter is a promoter associated with PTPRC or CCR7.

Also provided are methods of alleviating a disease, disorder, or condition in a subject comprising: administering to the subject the stable T reg cells produced according to the methods described herein, wherein the disease, disorder, or condition comprises an immune disease or cancer.

The disclosure also provides for use of the stable T reg cells produced by the methods herein in a method for treating a disease, disorder, or condition in a subject, wherein the disease, disorder, or condition comprises an immune disease or cancer.

Also provided is a population of stable T reg cells selected from a population T reg cells comprising a biallelic genetic modification of an auxotrophy-inducing gene resulting in auxotrophy for an auxotrophic factor and one or more transgenes re-expressing the auxotrophy-inducing gene or one or more independent functional domains of the auxotrophy-inducing gene. In some embodiments, the auxotrophy-inducing gene is UMPS, the auxotrophic factor is uridine, the independent functional domains are selected from OPRT and ODC, and the one or more transgenes further comprise a nucleotide sequence encoding FOXP3 or a FOXP3-dependent expression control sequence and a nucleotide sequence encoding a cell naïveté-associated promoter or a gene associated with a cell naïveté-associated promoter, optionally wherein the cell naïveté-associated promoter is a promoter associated with PTPRC or CCR7.

Provided herein are methods of generating a population of cells having incorporated a first and a second expression cassette, wherein the method comprises (a) culturing in the presence of uridine a plurality of cells genetically engineered to be auxotrophic for uridine; (b) contacting the plurality of cells with a first expression construct and a second expression construct; and (c) withdrawing the uridine from the plurality of cells, thereby generating the population of cells having incorporated a first and a second expression cassette. In some embodiments, the first expression construct comprises a first expression cassette comprising a nucleotide sequence encoding a first payload and a second expression cassette comprising a nucleotide sequence encoding a first independent functional domain of UMPS. The second expression construct can comprise a third expression cassette comprising a nucleotide sequence encoding a second payload and a fourth expression cassette comprising a nucleotide sequence encoding a second independent functional domain of UMPS. Thus, the first and the second expression constructs can comprise four expression cassettes, the expression cassettes including nucleotide sequences encoding the first and second independent functional domains of UMPS, and the remaining expression cassettes encoding one or more payload. In some embodiments, the methods comprise introducing additional expression constructs and/or expression constructs comprising nucleotide sequences encoding additional payloads.

In some embodiments, the first expression construct is a homologous recombination construct targeting a specific genetic locus. In some embodiments, the second expression construct is a homologous recombination construct targeting a specific genetic locus. The specific genetic locus can be a safe harbor locus. An example of a safe harbor locus is CCR5.

The plurality of cells genetically engineered to be auxotrophic for uridine can be UMPS knockout cells. In some embodiments, the plurality of cells is genetically engineered to be UMPS knockdown cells.

In some embodiments, the plurality of cells is derived from progenitor cells, e.g., pluripotent stem cells.

In some embodiments, the nucleotide sequence encoding the first payload is under the transcriptional control of a tissue-specific promoter. In some embodiments, the nucleotide sequence encoding the second payload is under the transcriptional control of a tissue-specific promoter. In some embodiments, the nucleotide sequence encoding the first payload and the nucleotide sequence encoding the second payload are each under the transcriptional control of a tissue-specific promoter.

In some embodiments, the nucleotide sequence encoding the first independent functional domain of UMPS is under the transcriptional control of a constitutive promoter. In some embodiments, the nucleotide sequence encoding the second independent functional domain of UMPS is under the transcriptional control of a constitutive promoter. In some embodiments, the nucleotide sequence encoding the first and the second independent functional domains of UMPS are under the transcriptional control of a constitutive promoter. In such embodiments, the first and second independent functional domains of UMPS are constitutively expressed, allowing for cells having stably incorporated the first and the second expression constructs to survive in the absence of uridine.

In some embodiments, the first and the second independent functional domains of UMPS are independently selected from OPRT and ODC.

In some embodiments, the methods described herein further comprise differentiating the cells in vitro to a desired cell type.

In some embodiments, the tissue-specific promoter is a megakaryocyte-specific promoter and the desired cell type is a megakaryocyte.

In some embodiments, differentiating the cells to the desired cell type leads to expression of the first payload, the second payload, or the first payload and the second payload, which can have tissue-specific promoter(s) corresponding to, i.e., upregulated in, the desired cell type.

Also provided herein are populations of cells comprising a first and a second expression cassette generated by the methods described herein.

Also provided are engineered cells comprising a knockout of an auxotrophy-inducing gene, a first expression construct, and a second expression construct, wherein the first expression construct and the second expression construct are stably integrated into the genome of the cell, and wherein the first expression construct and the second expression construct each comprise a nucleotide sequence encoding a first and a second independent functional domain of the auxotrophy-inducing gene.

In some embodiments of the engineered cells, the first and the second expression construct are integrated into the genome of the cells by homologous recombination.

Also provided are methods of generating megakaryocytes in vitro comprising (a) culturing in the presence of an auxotrophic factor a plurality of cells genetically engineered to be auxotrophic for the auxotrophic factor; (b) differentiating the cells to megakaryocytes; and (c) withdrawing the auxotrophic factor.

The methods of generating platelets in vitro can comprise starting with a plurality of cells comprising progenitor cells, e.g., pluripotent stem (PS) cells. The plurality of cells comprising PS cells can comprise UMPS knockout cells. The methods and/or cells wherein the auxotrophy-inducing gene is UMPS can comprise the use of uridine as an auxotrophic factor. Accordingly, withdrawing the uridine causes proliferative cells lacking the first and/or second independent functional domain of UMPS to die or to fail to propagate.

In some embodiments, the differentiated megakaryocytes generate platelets. The differentiated megakaryocytes can generate platelets in vitro. The platelets persist after withdrawing the auxotrophic factor, e.g., uridine. In some embodiments, a substantially pure population of platelets is generated. The substantially pure population of platelets can be devoid of nucleated cells, proliferative cells, megakaryocytes, pluripotent cells, and/or other cell types.

Accordingly, also provided herein are compositions comprising a substantially pure population of platelets generated by the methods provided herein.

Also provided are substantially pure populations of platelets generated in vitro from a plurality of cells genetically engineered to be auxotrophic.

Also provided herein are methods of generating a population of engineered platelets comprising: (a) culturing in the presence of an auxotrophic factor a plurality of cells genetically engineered to be auxotrophic for the auxotrophic factor, the plurality of cells having a knockout of an auxotrophy-inducing gene; (b) contacting the plurality of cells with a first expression construct and a second expression construct; and (c) withdrawing the auxotrophic factor from the plurality of cells.

In some embodiments, the first expression construct comprises a first expression cassette comprising a nucleotide sequence encoding a first payload and a second expression cassette comprising a nucleotide sequence encoding a first independent functional domain of the auxotrophy-inducing gene. In some embodiments, the second expression construct comprises a third expression cassette comprising a nucleotide sequence encoding a second payload and a fourth expression cassette comprising a nucleotide sequence encoding a second independent functional domain of the auxotrophy-inducing gene.

In some embodiments, the first expression construct, the second expression construct, or the first and the second expression construct can be homologous recombination construct(s) targeting a specific genetic locus. The specific genetic locus can be a safe harbor locus. The safe harbor locus can be CCR5.

In some embodiments, the auxotrophy-inducing gene is UMPS and the auxotrophic factor is uridine. Hence, the first and the second independent functional domains can be selected from UMPS independent functional domains OPRT and ODC.

In some embodiments, the plurality of cells is derived from progenitor cells, e.g., pluripotent stem cells.

In some embodiments, the nucleotide sequence encoding the first payload is under the transcriptional control of a tissue-specific promoter. In some embodiments, the nucleotide sequence encoding the second payload is under the transcriptional control of a tissue-specific promoter. In some embodiments, the nucleotide sequence encoding the first payload and the nucleotide sequence encoding the second payload are each under the transcriptional control of a tissue-specific promoter.

In some embodiments, the nucleotide sequence encoding the first independent functional domain is under the transcriptional control of a constitutive promoter. In some embodiments, the nucleotide sequence encoding the second independent functional domain is under the transcriptional control of a constitutive promoter. In some embodiments, the nucleotide sequence encoding the first and the second independent functional domains are each under the transcriptional control of a constitutive promoter.

Some embodiments of the methods of generating a population of engineered platelets further comprise differentiating the cells in vitro to a desired cell type. The tissue-specific promoter can be, for example, a megakaryocyte-specific promoter and the desired cell type can be a megakaryocyte. Differentiating the cells to the desired cell type can lead to expression of the first, the second, or the first payload and the second payload.

In some embodiments, megakaryocytes produced according to the methods provided herein produce platelets.

In some embodiments, the platelets are loaded with the first, the second, or the first and the second payload.

In some embodiments, differentiating the cells in vitro is performed in the presence of the auxotrophic factor. In some embodiments, the differentiated platelets do not express the first and the second independent functional domain.

In some embodiments, cells can be selected for by further culturing the cells with 5-FOA. The presence of 5-FOA can eliminate any residual non-edited cells, e.g., any residual PS cells, in the presence of the auxotrophic factor, e.g., uridine.

Some embodiments of generating a population of engineered platelets further comprise withdrawing the auxotrophic factor after differentiating the cells, wherein remaining nucleated, proliferating cells die or fail to propagate upon withdrawal of the auxotrophic factor.

Also provided herein are engineered cells comprising a knockout of UMPS, a first expression construct and a second expression construct, wherein the first expression construct and the second expression construct are stably integrated into the genome of the cell, and wherein the first expression construct and the second expression construct each comprise a nucleotide sequence encoding a first and a second independent functional domain of UMPS selected from OPRT and ODC. The first and the second expression construct can integrated into the genome of the cell by homologous recombination. In some embodiments, the first expression construct and the second expression construct each comprise homology arms targeting to a specific genetic locus. The specific genetic locus can be a safe harbor locus. The safe harbor locus can be CCR5 and the homology arms can be targeted to the CCR5 locus.

In some embodiments, the engineered cell comprises a first expression construct comprising an expression cassette further comprising a nucleotide sequence encoding a first payload.

In some embodiments, the engineered cell comprises a second expression construct comprising an expression cassette further comprising a nucleotide sequence encoding a second payload.

In some embodiments, the expression cassette further comprising the nucleotide sequence encoding a payload comprises a nucleotide sequence encoding an antisense RNA, an siRNA, an aptamer, a microRNA mimic, an anti-miR, a synthetic mRNA, or a polypeptide.

In some embodiments, the engineered cell comprises a first expression construct comprising a nucleotide sequence encoding a first payload and a second expression construct comprising a nucleotide sequence encoding a second payload.

The engineered cell can be derived from or differentiated from a progenitor cell, e.g., a pluripotent stem cell. In some embodiments, the engineered cell can be derived from or differentiated from a progenitor cell, e.g., a pluripotent stem cell cultured in vitro.

Also provided are engineered cells for use in methods of generating engineered platelets. In some embodiments, the engineered platelets can be loaded with the first payload, the second payload, or the first and the second payload.

Also provided are compositions comprising substantially pure populations of platelets prepared in vitro from cells engineered to be UMPS knockout cells. In some embodiments, the substantially pure population of platelets can be devoid or substantially devoid of nucleated or proliferative cells, such that any remaining nucleated or proliferative cells are senescent, dead, non-functional, non-proliferative, non-viable, and/or are present in sufficiently low numbers as to be effectively non-present.

In some embodiments, the substantially pure populations of platelets can be used in methods of treating a subject, the methods comprising administering the platelets to the subject.

In some embodiments, the substantially pure populations of platelets can be used in methods of delivering a therapeutic payload to a subject in need thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 is a schematic of an example process using split auxotrophic selection for optimizing expression vectors for use in PS cell-derived engineered megakaryocytes.

FIG. 2 is a schematic of an example process using uridine auxotrophy-based selection methods to generate platelets for in vivo applications from UMPS knockout (KO) pluripotent stem (PS) cells which have been differentiated in vitro to megakaryocytes (MKs).

FIG. 3 is a schematic of an example process using split auxotrophy to produce engineered platelets in vitro from pluripotent stem (PS) cells.

### DETAILED DESCRIPTION

### I. Introduction

Recent advances allow the precise modification of the genome of human cells. This genetic engineering enables a wide range of applications, but also requires new methods to control cell behavior. An alternative control system for cells is auxotrophy that can be engineered by targeting a gene in metabolism. The approach described herein of genetically engineering auxotrophy by disruption of a central gene of metabolism is an alternative paradigm to create an external control mechanism over cell function which has not been explored for human cells.

Auxotrophy has previously been engineered in microorganisms, e.g., towards an unnatural substance by introduction of an engineered gene circuit (see, Kato, Y. (2015) An engineered bacterium auxotrophic for an unnatural amino acid: a novel biological containment system. PeerJ 3, e1247, which is hereby incorporated by reference in its entirety) or towards pyrimidines by knockout of a bacterial gene (see, Steidler et al. (2003) Nat. Biotechnol. 21, 785-789, which is hereby incorporated by reference in its entirety). The latter concept is appealing, since it relies on the knockout of a gene instead of the introduction of complex expression cassettes, which impedes the cell from reversing the genetic modification or the development of resistance mechanisms, and therefore addresses this challenge of alternative systems. The fact that pyrimidine nucleosides and nucleotides play an important role in a wide array of cellular processes, including DNA and RNA synthesis, energy transfer, signal transduction and protein modification (see, van Kuilenburg, A. B. P. and Meinsma, R. (2016). Biochem. Biophys. Acta-Mol. Basis Dis. 1862, 1504-1512, which is hereby incorporated by reference in its entirety) makes their synthesis pathway a theoretically appealing target.

Human cells are naturally auxotrophic for certain compounds like amino acids that they have to acquire, either from external sources or symbiotic organisms (See, Murray, P. J. (2016). Nat. Immunol. 17, 132-139, which is hereby incorporated by reference in its entirety). Additionally, auxotrophy is a natural mechanism to modulate the function of immune cells, e.g. by differential supply or depletion of the metabolite that the cells are auxotrophic for (See, Grohmann et al., (2017). Cytokine Growth Factor Rev. 35, 37-45, which is hereby incorporated by reference in its entirety). Cellular auxotrophy also plays an important role in mechanisms of defense against malignant growth, e.g., in the case of macrophages that inhibit tumor growth by scavenging arginine (Murray, 2016). In addition, several malignant cell types have been shown to be auxotrophic for certain metabolites (see, Fung, M. K. L. and Chan, G. C. F. (2017). J. Hematol. Oncol. 10, 144, which is hereby incorporated by reference in its entirety), which is exploited by the therapeutic depletion of asparagine for the treatment of leukemia patients (See, Hill et al., (1967). JAMA 202, 882).

In addition to the previously developed containment strategies for microorganisms, the approach described herein using gene editing based on Cas9 ribonucleoprotein (RNP)/rAAV6 allows for highly efficient engineering of a primary and therapeutically relevant human cell type. Auxotrophy and resistance to 5-FOA are inherent to all cells with complete disruption of the UMPS gene, but to show proof-of-concept, the identification of the populations was facilitated with bi-allelic knockout by targeted integration of selection markers. The recent development of methods that allow the efficient targeted modification of primary human cells (see Bak, Rasmus O., et al. CRISPR/Cas9 and AAV6.” Elife 6 (2017): e27873; Bak, Rasmus O., et al. “Correction: Multiplexed genetic engineering of human hematopoietic stem and progenitor cells using CRISPR/Cas9 and AAV6.” Elife 7 (2018): e43690; Bak, Rasmus O., Daniel P. Dever, and Matthew H. Porteus. “CRISPR/Cas9 genome editing in human hematopoietic stem cells.” Nature protocols 13.2 (2018): 358; and Porteus, Matthew H., and David Baltimore. “Chimeric nucleases stimulate gene targeting in human cells.” Science 300.5620 (2003): 763-763; each of which is hereby incorporated by reference in its entirety) together with the establishment of metabolic auxotrophy lays the foundation for further development of therapeutic approaches in settings where the use of human cells is necessary, e.g., in the use of stem cells or stem-cell derived tissues or other autologous somatic cells with specific effector functions and reduced immunogenicity. Notably, constructs and reagents have been used that would facilitate expedited clinical translation, e.g., selection markers tNGFR and tEGFR in the targeting constructs, which avoid immunogenicity, and uridine supplied in the in vivo model using its FDA-approved prodrug.

Engineered mechanisms to control cell function have the additional challenge of selecting an entirely pure population of cells that express the proteins mediating the control mechanism. The possibility of selecting the engineered cells by rendering them resistant to a cytotoxic agent is particularly appealing since it can substantially increase efficiency by allowing the creation of a highly pure population of cells that can be controlled using a non-toxic substance, and the removal of a gene crucial for the function of a vital metabolic pathway prevents cells from developing escape mechanisms. Therefore, this method offers several advantages over existing control mechanisms in settings where genetic instability and the risk of malignant transformation play a role and where even small numbers of cells that escape their containment can have disastrous effects, e.g., in the use of somatic or pluripotent stem cells.

This concept has been explored for microorganisms (Steidler et al., 2003) and has been broadly used as a near universal research tool by yeast geneticists. It would be particularly powerful in mammalian cells if it is created by knockout of a gene instead of by introduction of a complex control mechanism, and if the auxotrophy is towards a non-toxic compound that is part of the cell's endogenous metabolism. This could be achieved by disruption of an essential gene in a metabolic pathway, allowing the cell to function only if the product of that pathway is externally supplied and taken up by the cell from its environment. Furthermore, if the respective gene is also involved in the activation of a cytotoxic agent, the gene knockout (KO) would render the cells resistant to that drug, thereby enabling the depletion of non-modified cells and purification of the engineered cells in a cell population. Several monogenic inborn errors of metabolism can be treated by supply of a metabolite and can therefore be seen as models of human auxotrophy.

In certain embodiments, auxotrophy is introduced to human cells by disrupting UMPS in the de-novo pyrimidine synthesis pathway through genome editing. This makes the cell's function dependent on the presence of exogenous uridine. Furthermore, this abolishes the cell's ability to metabolize 5-fluoroorotic acid into 5-FU, which enables the depletion of remaining cells with intact UMPS alleles. The ability to use a metabolite to influence the function of human cells by genetically engineered auxotrophy and to deplete other cells provides for the development of this approach for a range of applications where a pure population of controllable cells is necessary.

One example of an auxotrophy is hereditary orotic aciduria, in which mutations in the UMPS gene lead to a dysfunction that can be treated by supplementation with high doses of uridine (Fallon et al., 1964). Transferring this concept to a cell type of interest, genetic engineering was used to knock out the UMPS gene in human cells which makes the cells auxotrophic to uridine and resistant to 5-fluoroorotic acid (5-FOA). UMPS^{-/-} cell lines and primary cells are shown herein to survive and proliferate only in the presence of uridine in vitro, and that UMPS engineered cell proliferation is inhibited without supplementation of uridine in vivo. Furthermore, the cells can be selected from a mixed population by culturing in the presence of 5-FOA.

In certain embodiments, a tissue-specific promoter may be inserted into the UMPS locus to control expression of the gene in a progenitor cell, wherein differentiation of the progenitor cell into the type of tissue associated with the inserted promoter results in expression of the UMPS gene.

### II. Compositions of the Present Disclosure

Disclosed herein are some embodiments of methods and compositions for use in cell selection methods and selective expression of a transgene. In some instances, the methods comprise delivery of a construct, potentially including a transgene encoding a therapeutic factor or including a tissue-specific promoter, to cells in a manner that renders the cells auxotrophic, and the differentiated cells prototrophic (i.e., capable of synthesizing all nutrients or factors needed for survival and/or growth), and that can provide improved efficacy, potency, and/or safety of gene therapy through transgene expression.

Delivery of the construct to a specific auxotrophy-inducing locus creates an auxotrophic cell, for example, through disruption or knockout of a gene or downregulation of a gene's activity, that is now dependent on continuous administration of an auxotrophic factor for growth and reproduction. In some instances, the methods comprise employing nuclease systems targeting the auxotrophy-inducing locus, vectors for inserting the constructs disclosed herein, kits, and methods of using such systems, templates and vectors to produce modified cells that are auxotrophic and capable of expressing the introduced construct.

Also disclosed herein, in some embodiments, are methods, compositions and kits for use of the modified cells, including pharmaceutical compositions, therapeutic methods, and methods of administration of auxotrophic factors to control—increase, decrease or cease—the growth and reproduction of the modified cells and to control the expression of the transgene and to control levels of the therapeutic factor.

In some instances, delivery of the construct to the desired locus can be accomplished through methods such as homologous recombination. As used herein, “homologous recombination (HR)” refers to insertion of a nucleotide sequence during repair of double-strand breaks in DNA via homology-directed repair mechanisms. This process uses a “donor” molecule or “donor template” with homology to nucleotide sequence in the region of the break as a template for repairing a double-strand break. The presence of a double-stranded break facilitates integration of the donor sequence. The donor sequence may be physically integrated or used as a template for repair of the break via homologous recombination, resulting in the introduction of all or part of the nucleotide sequence. This process is used by a number of different gene editing platforms that create the double-strand break, such as meganucleases, such as zinc finger nucleases (ZFNs), transcription activator-like effector nucleases (TALENs), and the CRISPR-Cas9 gene editing systems.

In some embodiments, genes are delivered to two or more loci, for example, for the expression of multiple therapeutic factors, or for the introduction of a second gene that acts as a synthetic regulator or that acts to bias the modified cells towards a certain lineage (e.g. by expressing a transcription factor from the second locus). In some embodiments, genes are delivered to two or more auxotrophy-inducing loci. For example, a different gene or a second copy of the same gene is delivered to a second auxotrophy-inducing locus.

In some embodiments, the cell is auxotrophic because the cell no longer has the ability to produce the auxotrophic factor. As used herein, a “cell”, “modified cell” or “modified host cell” refers to a population of cells descended from the same cell, with each cell of the population having a similar genetic make-up and retaining the same modification.

In some embodiments, the auxotrophic factor comprises one or two or more nutrients, enzymes, altered pH, altered temperature, non-organic molecules, non-essential amino acids, or altered concentrations of a moiety (compared to normal physiologic concentrations), or combinations thereof. All references to auxotrophic factor herein contemplate administration of multiple factors. In any of the embodiments described herein, the auxotrophic factor is a nutrient or enzyme that is neither toxic nor bioavailable in the subject in concentrations sufficient to sustain the modified host cell, and it is to be understood that any references to “auxotrophic factor” throughout this application may include reference to a nutrient or enzyme.

In some instances, if the modified cell is not continuously supplied with the auxotrophic factor, the cell ceases proliferation or dies. In some instances, the modified cell provides a safety switch that decreases the risks associated with other cell-based therapies that include oncogenic transformation.

The methods and compositions disclosed herein provide a number of advantages, for example: consistent results and conditions due to integrating into the same locus rather than random integration such as with lentivectors; constant expression of transgene because areas with native promoters or enhancers or areas that are silenced are avoided; a consistent copy number of integration, 1 or 2 copies, rather than a Poisson distribution; and limited chance of oncogenic transformation. In some instances, the modified cells of the present disclosure are less heterogeneous than a product engineered by lentivector or other viral vector.

A. Therapeutic Factors

The following embodiments provide conditions to be treated by producing a therapeutic factor in an auxotrophic host cell.

Clotting disorders, for example, are fairly common genetic disorders where factors in the clotting cascade are absent or have reduced function due to a mutation. These include hemophilia A (factor VIII deficiency), hemophilia B (factor IX deficiency), or hemophilia C (factor XI deficiency).

Alpha-1 antitrypsin (A1AT) deficiency is an autosomal recessive disease caused by defective production of alpha 1-antitrypsin which leads to inadequate A1AT levels in the blood and lungs. It can be associated with the development of chronic obstructive pulmonary disease (COPD) and liver disorders.

Type I diabetes is a disorder in which immune-mediated destruction of pancreatic beta cells results in a profound deficiency of insulin production. Complications include ischemic heart disease (angina and myocardial infarction), stroke and peripheral vascular disease, diabetic retinopathy, diabetic neuropathy, and diabetic nephropathy, which may result in chronic kidney disease requiring dialysis.

Antibodies are secreted protein products used for neutralization or clearance of target proteins that cause disease as well as highly selective killing of certain types of cells (e.g. cancer cells, certain immune cells in autoimmune diseases, cells infected with virus such as human immunodeficiency virus (HIV), RSV, Flu, Ebola, CMV, and others). Antibody therapy has been widely applied to many human conditions including oncology, rheumatology, transplant, and ocular disease. In some instances, the therapeutic factor encoded by the compositions disclosed herein is an antibody used to prevent or treat conditions such as cancer, infectious diseases and autoimmune diseases. In certain embodiments, the cancer is treated by reducing the rate of growth of a tumor or by reducing the size of a tumor in the subject.

Monoclonal antibodies approved by the FDA for therapeutic use include Adalimumab, Bezlotoxumab, Avelumab, Dupilumab, Durvalumab, Ocrelizumab, Brodalumab, Reslizumab, Olaratumab, Daratumumab, Elotuzumab, Necitumumab, Infliximab, Obiltoxaximab, Atezolizumab, Secukinumab, Mepolizumab, Nivolumab, Alirocumab, Idarucizumab, Evolocumab, Dinutuximab, Bevacizumab, Pembrolizumab, Ramucirumab, Vedolizumab, Siltuximab, Alemtuzumab, Trastuzumab emtansine, Pertuzumab, Infliximab, Obinutuzumab, Brentuximab, Raxibacumab, Belimumab, Ipilimumab, Denosumab, Denosumab, Ofatumumab, Besilesomab, Tocilizumab, Canakinumab, Golimumab, Ustekinumab, Certolizumab pegol, Catumaxomab, Eculizumab, Ranibizumab, Panitumumab, Natalizumab, Catumaxomab, Bevacizumab, Omalizumab, Cetuximab, Efalizumab, Ibritumomab tiuxetan, Fanolesomab, Adalimumab, Tositumomab, Alemtuzumab, Trastuzumab, Gemtuzumab ozogamicin, Infliximab, Palivizumab, Necitumumab, Basiliximab, Rituximab, Votumumab, Sulesomab, Arcitumomab, Imiciromab, Capromab, Nofetumomab, Abciximab, Satumomab, and Muromonab-CD3. Bispecific antibody approved by the FDA for therapeutic use includes Blinatumomab. In some embodiments, the antibody is used to prevent or treat HIV or other infectious diseases. Antibodies for use in treatment of HIV include human monoclonal antibody (mAb) VRC-HIVMAB060-00-AB (VRC01); mAb VRC-HIVMAB080-00-AB (VRC01LS); mAb VRC-HIVMAB075-00-AB (VRC07-523LS); mAb F105; mAb C2F5; mAb C2G12; mAb C4E10; antibody UB-421 (targeting the HIV-1 receptor on the CD4 molecule (domain 1) of T-lymphocytes and monocytes); Ccr5mab004 (Human Monoclonal IgG4 antibody to Ccr5); mAb PGDM1400; mAb PGT121 (recombinant human IgG1 monoclonal antibodies that target a V1V2 (PGDM1400) and a V3 glycan-dependent (PGT121) epitope region of the HIV envelope protein); KD-247 (a humanized monoclonal antibody); PRO 140 (a monoclonal CCR5 antibody); mAb 3BNC117; and PG9 (anti-HIV-1 gp120 monoclonal antibody).

Therapeutic RNAs include antisense, siRNAs, aptamers, microRNA mimics/anti-miRs and synthetic mRNA, and some of these can be expressed by transgenes.

Lysosomal storage disorders (“LSDs”) are inherited metabolic diseases that are characterized by an abnormal build-up of various toxic materials in the body's cells as a result of enzyme deficiencies. There are nearly 50 of these disorders altogether, and they affect different parts of the body, including the skeleton, brain, skin, heart, and central nervous system. Common examples include Sphingolipidoses, Farber disease (ASAH1 deficiency), Krabbe disease (galactosylceramidase or GALC deficiency), Galactosialidosis, Gangliosidoses, Alpha-galactosidase, Fabry disease (a-galactosidase deficiency—GLA, or agalsidase alpha/beta), Schindler disease (alpha-NAGA deficiency), GM1 gangliosidosis, GM2 gangliosidoses (beta-hexosaminidase deficiency), Sandhoff disease (hexosaminidase-B deficiency), Tay-Sachs disease (hexosaminidase-A deficiency), Gaucher's disease Type 1/2/3 (glucocerebrosidase deficiency-gene name: GBA), Wolman disease (LAL deficiency), Niemann-Pick disease type A/B (sphingomyelin phosphodiesterase 1 deficiency—SMPD1 or acid sphingomyelinase), Sulfatidosis, Metachromatic leukodystrophy, Hurler syndrome (alpha-L iduronidase deficiency—IDUA), Hunter syndrome or MPS2 (iduronate-2-sulfatase deficiency-idursulfase or IDS), Sanfilippo syndrome, Morquio, Maroteaux-Lamy syndrome, Sly syndrome (β-glucuronidase deficiency), Mucolipidosis, I-cell disease, Lipidosis, =Neuronal ceroid lipofuscinoses, Batten disease (tripeptidyl peptidase-1 deficiency), Pompe (alglucosidase alpha deficiency), hypophosphatasia (asfotase alpha deficiency), MPS1 (laronidase deficiency), MPS3A (heparin N-sulfatase deficiency), MPS3B (alpha-N-acetylglucosaminidase deficiency), MPS3C (heparin-a-glucosaminide N-acetyltransferase deficiency), MPS3D (N-acetylglucosamine 6-sulfatase deficiency), MPS4 (elosulfase alpha deficiency), MPS6 (glasulfate deficiency), MPS7 (B-glucoronidase deficiency), phenylketonuria (phenylalanine hydroxylase deficiency), and MLD (arylsulphatase A deficiency). Collectively LSDs have an incidence in the population of about 1 in 7000 births and have severe effects including early death. While clinical trials are in progress on possible treatments for some of these diseases, there is currently no approved treatment for many LSDs. Current treatment options for some but not all LSDs include enzyme replacement therapy (ERT). ERT is a medical treatment which replaces an enzyme that is deficient or absent in the body. In some instances, this is done by giving the patient an intravenous (IV) infusion of a solution containing the enzyme.

Disclosed herein, in some embodiments, are methods of treating a LSD in an individual in need thereof, the method comprising providing to the individual enzyme replacement therapy using the compositions disclosed herein, such as the cells selected using an embodiment of a method described herein. In some instances, the method comprises a modified host cell ex vivo, comprising a construct encoding an enzyme integrated at an auxotrophy-inducing locus, wherein said modified host cell is auxotrophic for an auxotrophic factor and capable of expressing the enzyme that is deficient in the individual, thereby treating the LSD in the individual. In some instances, the auxotrophy-inducing locus is within a gene in Table 2 or within a region that controls expression of a gene in Table 2. In some instances, the auxotrophy-inducing locus is within a gene encoding uridine monophosphate synthetase (UMPS). In some instances, the auxotrophic factor is uridine. In some instances, the auxotrophy-inducing locus is within a gene encoding holocarboxylase synthetase (HLCS). In some instances, the auxotrophic factor is biotin. In some instances, the auxotrophy-inducing locus is within a gene encoding asparagine synthetase. In some instances, the auxotrophic factor is asparagine. In some instances, the auxotrophy-inducing locus is within a gene encoding aspartate transaminase. In some instances, the auxotrophic factor is aspartate. In some instances, the auxotrophy-inducing locus is within a gene encoding alanine transaminase. In some instances, the auxotrophic factor is alanine. In some instances, the auxotrophy-inducing locus is within a gene encoding cystathionine beta synthase. In some instances, the auxotrophic factor is cysteine. In some instances, the auxotrophy-inducing locus is within a gene encoding cystathionine gamma-lyase. In some instances, the auxotrophic factor is cysteine. In some instances, the auxotrophy-inducing locus is within a gene encoding glutamine synthetase. In some instances, the auxotrophic factor is glutamine. In some instances, the auxotrophy-inducing locus is within a gene encoding serine hydroxymethyltransferase. In some instances, the auxotrophic factor is serine or glycine. In some instances, the auxotrophy-inducing locus is within a gene encoding glycine synthase. In some instances, the auxotrophic factor is glycine. In some instances, the auxotrophy-inducing locus is within a gene encoding phosphoserine transaminase. In some instances, the auxotrophic factor is serine. In some instances, the auxotrophy-inducing locus is within a gene encoding phosphoserine phosphatase. In some instances, the auxotrophic factor is serine. In some instances, the auxotrophy-inducing locus is within a gene encoding phenylalanine hydroxylase. In some instances, the auxotrophic factor is tyrosine. In some instances, the auxotrophy-inducing locus is within a gene encoding argininosuccinate synthetase. In some instances, the auxotrophic factor is arginine. In some instances, the auxotrophy-inducing locus is within a gene encoding argininosuccinate lyase. In some instances, the auxotrophic factor is arginine. In some instances, the auxotrophy-inducing locus is within a gene encoding dihydrofolate reductase. In some instances, the auxotrophic factor is folate or tetrahydrofolate.

Further disclosed herein, in some embodiments, are methods of treating a disease or disorder in an individual in need thereof, the method comprising providing to the individual protein replacement therapy using the compositions disclosed herein. In some instances, the method comprises a modified host cell ex vivo, comprising a construct encoding a protein integrated at an auxotrophy-inducing locus, wherein said modified host cell is auxotrophic for an auxotrophic factor and capable of expressing the protein that is deficient in the individual, thereby treating the disease or disorder in the individual. In some instances, the auxotrophy-inducing locus is within a gene in Table 2 or within a region that controls expression of a gene in Table 2. In some instances, the auxotrophy-inducing locus is within a gene encoding uridine monophosphate synthetase (UMPS). In some instances, the auxotrophic factor is uridine. In some instances, the auxotrophy-inducing locus is within a gene encoding holocarboxylase synthetase (HLCS). In some instances, the auxotrophic factor is biotin. In some instances, the disease is Friedreich's ataxia, and the protein is frataxin. In some instances, the disease is hereditary angioedema and the protein is C1 esterase inhibitor (e.g., HAEGAARDA® subcutaneous injection). In some instances, the disease is spinal muscular atrophy and the protein is SMN1.

B. Auxotrophic Cell Populations

Disclosed herein, in some embodiments, are compositions comprising cells, preferably human cells, that are genetically engineered to be auxotrophic. Auxotrophy may be induced through insertion of a construct encoding an auxotrophy-inducing gene, or in some embodiments, an auxotrophic factor, and/or a therapeutic factor at an auxotrophy-inducing locus and are capable of expressing the therapeutic factor. Animal cells, mammalian cells, preferably human cells, modified ex vivo, in vitro, or in vivo are contemplated. Also included are cells of other primates; mammals, including commercially relevant mammals, such as cattle, pigs, horses, sheep, cats, dogs, mice, rats; birds, including commercially relevant birds such as poultry, chickens, ducks, geese, and/or turkeys.

In some embodiments, a tissue-specific promoter is inserted into the auxotrophy-inducing locus. For example, an auxotrophic factor, a re-expressed auxotrophy-inducing gene, and/or a therapeutic factor is expressed only when the progenitor cell is differentiated into the tissue associated with the tissue-specific promoter.

In some embodiments, the progenitor cell is an embryonic stem cell, a stem cell, a progenitor cell, a pluripotent stem cell, an induced pluripotent stem (iPS) cell, a somatic stem cell, a differentiated cell, a mesenchymal stem cell or a mesenchymal stromal cell, a neural stem cell, a hematopoietic stem cell or a hematopoietic progenitor cell, an adipose stem cell, a keratinocyte, a skeletal stem cell, a muscle stem cell, a fibroblast, an NK cell, a B-cell, a T cell, or a peripheral blood mononuclear cell (PBMC). For example, the cell may be engineered to express a CAR, thereby creating a CAR-T cell.

To prevent immune rejection of the modified cells when administered to a subject, the cells to be modified are preferably derived from the subject's own cells. Thus, preferably the mammalian cells are from the subject to be treated with the modified cells. In some instances, the mammalian cells are autologous cells. In some instances, the mammalian cells are allogeneic cells. In some instances, modified T cells can be further modified to prevent graft versus host disease, for example, by inactivating the T cell receptor locus. In some instances, modified cells can further be modified to be immune-inert, for example, by deleting B2M to remove MHC class I on the surface of the cell, or by deleting B2M and then adding back an HLA-G-B2M fusion to the surface to prevent NK cell rejection of cells that do not have MHC Class I on their surface.

The cell lines may include stem cells that were maintained and differentiated using the techniques below as shown in U.S. Pat. No. 8,945,862, which is hereby incorporated by reference in its entirety. In some embodiments, the stem cell is not a human embryonic stem cell. Furthermore, the cell lines may include stem cells made by the techniques disclosed in WO 2003/046141 or Chung et al. Cell Stem Cell, February 2008, Vol. 2, pages 113-117, each of which is hereby incorporated by reference in its entirety.

For example, the cells may be stem cells isolated from the subject for use in a regenerative medical treatment in any of epithelium, cartilage, bone, smooth muscle, striated muscle, neural epithelium, stratified squamous epithelium, and ganglia. Disease that results from the death or dysfunction of one or a few cell types, such as Parkinson's disease and juvenile onset diabetes, are also commonly treated using stem cells (see, Thomson et al., Science, 282:1145-1147, 1998, which is hereby incorporated by reference in its entirety).

In some embodiments, cells are harvested from the subject and modified according to the methods disclosed herein, which can include selecting certain cell types, optionally expanding the cells and optionally culturing the cells, and which can additionally include selecting cells that contain the construct integrated into the auxotrophy-inducing locus.

C. Constructs

Therapeutic entities encoded by the genome of the modified host cell may cause therapeutic effects, such as molecule trafficking, inducing cell death, recruitment of additional cells, or cell growth. In some embodiments, the therapeutic effect is expression of a therapeutic protein. In some embodiments, the therapeutic effect is induced cell death, including cell death of a tumor cell.

In some embodiments, after a nuclease system is used to cleave DNA, homology-directed repair mechanisms may be used to insert a construct during their repair of the break in the DNA. In some instances, the construct template comprises a region that is homologous to nucleotide sequence in the region of the break so that the donor template hybridizes to the region adjacent to the break and is used as a template for repairing the break.

In some embodiments, the construct is flanked on both sides by nucleotide sequences homologous to a fragment of the auxotrophy-inducing locus or the complement thereof. In some instances, the construct is single stranded, double stranded, a plasmid or a DNA fragment. In some instances, plasmids comprise elements necessary for replication, including a promoter and optionally a 3′ UTR.

Further disclosed herein are vectors comprising (a) one or more nucleotide sequences homologous to a fragment of the auxotrophy-inducing locus, or homologous to the complement of said auxotrophy-inducing locus, which can be a viral vector, such as a retroviral, lentiviral (both integration competent and integration defective lentiviral vectors), adenoviral, adeno-associated viral or herpes simplex viral vector. Viral vectors may further comprise genes necessary for replication of the viral vector.

In some embodiments, the targeting construct comprises: (1) a viral vector backbone, e.g. an AAV backbone, to generate virus; (2) arms of homology to the target site of at least 200 bp but ideally 400 bp on each side to assure high levels of reproducible targeting to the site (see, Porteus, Annual Review of Pharmacology and Toxicology, Vol. 56:163-190 (2016); which is hereby incorporated by reference in its entirety); (3) potentially a transgene encoding a therapeutic factor and capable of expressing the therapeutic factor; (4) an expression control sequence operably linked to the transgene; and optionally (5) an additional marker gene to allow for enrichment and/or monitoring of the modified host cells.

Suitable marker genes are known in the art and include Myc, HA, FLAG, GFP, mCherry, truncated NGFR, truncated EGFR, truncated CD20, truncated CD19, as well as antibiotic resistance genes.

Any AAV known in the art can be used. In some embodiments the primary AAV serotype is AAV6.

In any of the preceding embodiments, the construct or vector comprises a nucleotide sequence homologous to a fragment of the auxotrophy-inducing locus, optionally any of the genes in Table 2 below, wherein the nucleotide sequence is at least 85, 88, 90, 92, 95, 98, or 99% identical to at least 200, 250, 300, 350, or 400 consecutive nucleotides of the auxotrophy-inducing locus; up to 400 nucleotides is usually sufficient to assure accurate recombination. Any combination of the foregoing parameters is envisioned, e.g. at least 85% identical to at least 200 consecutive nucleotides, or at least 88% identical to at least 200 consecutive nucleotides, or at least 90% identical to at least 200 consecutive nucleotides, or at least 92% identical to at least 200 consecutive nucleotides, or at least 95% identical to at least 200 consecutive nucleotides, or at least 98% identical to at least 200 consecutive nucleotides, or at least 99% identical to at least 200 consecutive nucleotides, or at least 85% identical to at least 250 consecutive nucleotides, or at least 88% identical to at least 250 consecutive nucleotides, or at least 90% identical to at least 250 consecutive nucleotides, or at least 92% identical to at least 250 consecutive nucleotides, or at least 95% identical to at least 250 consecutive nucleotides, or at least 98% identical to at least 250 consecutive nucleotides, or at least 99% identical to at least 250 consecutive nucleotides, or at least 85% identical to at least 300 consecutive nucleotides, or at least 88% identical to at least 300 consecutive nucleotides, or at least 90% identical to at least 300 consecutive nucleotides, or at least 92% identical to at least 300 consecutive nucleotides, or at least 95% identical to at least 300 consecutive nucleotides, or at least 98% identical to at least 300 consecutive nucleotides, or at least 99% identical to at least 300 consecutive nucleotides, or at least 85% identical to at least 350 consecutive nucleotides, or at least 88% identical to at least 350 consecutive nucleotides, or at least 90% identical to at least 350 consecutive nucleotides, or at least 92% identical to at least 350 consecutive nucleotides, or at least 95% identical to at least 350 consecutive nucleotides, or at least 98% identical to at least 350 consecutive nucleotides, or at least 99% identical to at least 350 consecutive nucleotides, or at least 85% identical to at least 400 consecutive nucleotides, or at least 88% identical to at least 400 consecutive nucleotides, or at least 90% identical to at least 400 consecutive nucleotides, or at least 92% identical to at least 400 consecutive nucleotides, or at least 95% identical to at least 400 consecutive nucleotides, or at least 98% identical to at least 400 consecutive nucleotides, or at least 99% identical to at least 400 consecutive nucleotides.

The disclosure also contemplates a system for targeting integration of a construct to an auxotrophy-inducing locus comprising a cas9 protein and a guide RNA.

The disclosure further contemplates a system for targeting integration of a construct to an auxotrophy-inducing locus comprising said donor template or vector and an endonuclease specific for said auxotrophy-inducing locus. The endonuclease can be, for example, a ZFN, TALEN, or meganuclease.

The inserted construct can also include other safety switches, such as a standard suicide gene into the locus (e.g. iCasp9) in circumstances where rapid removal of cells might be required due to acute toxicity. In some embodiments, differentiated cells selected for using the methods described herein are prototrophic upon in vitro differentiation, but can be made auxotrophic again thereafter (for example, prior to implantation into a subject for therapeutic application) by inserting a conditional safety switch such as a conditional destabilization domain, or ribozyme, as described herein, so that any engineered cell transplanted into a body can be eliminated by removal of an auxotrophic factor. This is especially important if the engineered cell has transformed into a cancerous cell.

In some instances, the donor polynucleotide or vector optionally further comprises an expression control sequence operably linked to said transgene. In some embodiments, the expression control sequence is a promoter or enhancer, an inducible promoter, a constitutive promoter, a tissue-specific promoter or expression control sequence, a posttranscriptional regulatory sequence or a microRNA (miRNA).

D. Nuclease Systems

In some embodiments, the compositions disclosed herein comprise nuclease systems targeting the auxotrophy-inducing locus. For example, the disclosure contemplates (a) a endonuclease that targets and cleaves DNA at said auxotrophy-inducing locus, or (b) a polynucleotide that encodes said endonuclease, including a vector system for expressing said endonuclease. As one example, the endonuclease is a TALEN that is a fusion protein comprising (i) a Transcription Activator Like Effector (TALE) DNA binding domain that binds to the auxotrophy-inducing locus, wherein the TALE DNA binding protein comprises a plurality of TALE repeat units, each TALE repeat unit comprising an amino acid sequence that binds to a nucleotide in a target sequence in the auxotrophy-inducing locus, and (ii) a DNA cleavage domain.

Also disclosed herein are CRISPR/Cas or CRISPR/Cpf1 system that targets and cleaves DNA at said auxotrophy-inducing locus that comprises (a) a Cas (e.g. Cas9) or Cpf1 polypeptide or a nucleic acid encoding said polypeptide, and (b) a guide RNA that hybridizes specifically to said auxotrophy-inducing locus, or a nucleic acid encoding said guide RNA. In nature, the Cas9 system is composed of a cas9 polypeptide, a crRNA, and a trans-activating crRNA (tracrRNA). As used herein, “cas9 polypeptide” refers to a naturally occurring cas9 polypeptide or a modified cas9 polypeptide that retains the ability to cleave at least one strand of DNA. The modified cas9 polypeptide can, for example, be at least 75%, 80%, 85%, 90%, or 95% identical to a naturally occurring Cas9 polypeptide. Cas9 polypeptides from different bacterial species can be used; S. pyogenes is commonly sold commercially. The cas9 polypeptide normally creates double-strand breaks but can be converted into a nickase that cleaves only a single strand of DNA (i.e. produces a “single stranded break”) by introducing an inactivating mutation into the HNH or RuvC domain. Similarly, the naturally occurring tracrRNA and crRNA can be modified as long as they continue to hybridize and retain the ability to target the desired DNA, and the ability to bind the cas9. The guide RNA can be a chimeric RNA, in which the two RNAs are fused together, e.g. with an artificial loop, or the guide RNA can comprise two hybridized RNAs. The meganuclease or CRISPR/Cas or CRISPR/Cpf1 system can produce a double stranded break or one or more single stranded breaks within the auxotrophy-inducing locus, for example, to produce a cleaved end that includes an overhang.

In some instances, the nuclease systems described herein, further comprises a construct as described herein.

Various methods are known in the art for editing nucleic acid, for example to cause a gene knockout or expression of a gene to be downregulated. For example, various nuclease systems, such as zinc finger nucleases (ZFN), transcription activator-like effector nucleases (TALEN), meganucleases, or combinations thereof are known in the art to be used to edit nucleic acid and may be used in the present disclosure. Meganucleases are modified versions of naturally occurring restriction enzymes that typically have extended or fused DNA recognition sequences.

The CRISPR/Cas system is detailed in, for example WO 2013/176772, WO 2014/093635 and WO 2014/089290; each of which is hereby incorporated by reference in its entirety. Its use in T cells is suggested in WO 2014/191518, which is hereby incorporated by reference in its entirety. CRISPR engineering of T cells is discussed in EP 3004349, which is hereby incorporated by reference in its entirety.

The time-limiting factor for generation of mutant (knock-out, knock-in, or gene replaced) cell lines was the clone screening and selection before development of the CRISPR/Cas9 platform. The term “CRISPR/Cas9 nuclease system” as used herein, refers to a genetic engineering tool that includes a guide RNA (gRNA) (also, “single guide RNA” (sgRNA)) sequence with a binding site for Cas9 and a targeting sequence specific for the area to be modified. The Cas9 binds the gRNA to form a ribonucleoprotein that binds and cleaves the target area. In certain embodiments, the gRNA/sgRNA is selected from one described in U.S. 62/669,848 filed May 10, 2018:

gRNA sequences, including protospacer-adjacent motifs (PAMs), are provided in Table 1:

TABLE 1
gRNA Sequences

| | | SEQ |
|---|---|---|
| gRNA ID | Nucleotide Sequence | ID NO: |
| UMPS-7 | GCCCCGCAGAUCGAUGUAGAGUUUUAGAGCUAG | 10 |
| | AAAUAGCAAGUUAAAAUAAGGCUAGUCCGUUAU | |
| | CAACUUGAAAAAGUGGCACCGAGUCGGUGCUUU | |
| | U | |
| UMPS-3 | CCCCGCAGAUCGAUGUAGAUGGG | 8 |
| UMPS-6 | GGCGGUCGCUCGUGCAGCUUUGG | 9 |

In addition to the CRISPR/Cas 9 platform (which is a type II CRISPR/Cas system), alternative systems exist including type I CRISPR/Cas systems, type III CRISPR/Cas systems, and type V CRISPR/Cas systems. Various CRISPR/Cas9 systems have been disclosed, including Streptococcus pyogenes Cas9 (SpCas9), Streptococcus thermophilus Cas9 (StCas9), Campylobacter jejuni Cas9 (CjCas9) and Neisseria cinerea Cas9 (NcCas9) to name a few. Alternatives to the Cas system include the Francisella novicida Cpf1 (FnCpf1), Acidaminococcus sp. Cpf1 (AsCpf1), and Lachnospiraceae bacterium ND2006 Cpf1 (LbCpf1) systems. Any of the above CRISPR systems may be used in methods to generate the cell lines disclosed herein. For example, the CRISPR system used may be the CRISPR/Cas9 system, such as the S. pyogenes CRISPR/Cas9 system.

E. Auxotrophic Factors

In some embodiments, disruption of a single gene causes the desired auxotrophy. In alternative embodiments, disruption of multiple genes produces the desired auxotrophy.

In some embodiments, the auxotrophy-inducing locus is a gene encoding a protein that produces an auxotrophic factor, which includes proteins upstream in the pathway for producing the auxotrophic factor.

In some embodiments described herein, the auxotrophy-inducing locus is the gene encoding uridine monophosphate synthetase (UMPS) (and the corresponding auxotrophic factor is uridine), or the gene encoding holocarboxylase synthetase (and the corresponding auxotrophic factor is biotin). In some embodiments, auxotrophy-inducing loci are selected from the following genes in Table 2. The genes of Table 2 were collated by selecting S. cerevisiae genes with a phenotype annotated as “Auxotrophy” downloaded with “Chemical” data from the yeast phenotype ontology database on the Saccharomyces genome database (SGD) (See, Cherry et al. 2012, Nucleic Acids Res. 40:D700-D705, which is hereby incorporated by reference in its entirety). These genes were converted into human homologues using the YeastMine® database or, in alternative embodiments, the Saccharocyces Genome Database (SGD). The genes are identified by their ENSEMBL gene symbol and ENSG identifier, which are found in the ENSEMBL database (www.ensembl.org). The first five zeroes of the ENSG identifiers (e.g., ENSG00000) have been removed.

TABLE 2
Auxotrophy-inducing loci

| Gene | ENSG(s) | Auxotrophic factor |
|---|---|---|
| AACS | 081760 | lysine |
| AADAT | 109576 | histidine |
| AASDHPPT | 149313 | lysine |
| AASS | 008311 | lysine |
| ACAT1 | 075239 | ergosterol |
| ACCS | 110455 | histidine |
| ACCSL | 205126 | histidine |
| ACO1 | 122729 | leucine |
| ACO2 | 100412 | leucine |
| ACSS3 | 111058 | lysine |
| ADSL | 239900 | adenine |
| ADSS | 035687 | adenine |
| ADSSL1 | 185100 | adenine |
| ALAD | 148218 | cysteine |
| ALAS1 | 023330 | cysteine |
| ALAS2 | 158578 | cysteine |
| ALDH1A1 | 165092 | pantothenic acid |
| ALDH1A2 | 128918 | pantothenic acid |
| ALDH1A3 | 184254 | pantothenic acid |
| ALDH1B1 | 137124 | pantothenic acid |
| ALDH2 | 111275 | pantothenic acid |
| AMD1 | 123505 | 0.25 mM spermine |
| ASL | 126522 | arginine |
| ASS1 | 130707 | arginine |
| ATF4 | 128272 | methionine |
| ATF5 | 169136 | methionine |
| AZIN1 | 155096 | 0.25 mM putrescine |
| AZIN2 | 142920 | 0.25 mM putrescine |
| BCAT1 | 060982 | valine, leucine |
| BCAT2 | 105552 | valine, leucine |
| CAD | 084774 | uridine |
| CBS | 160200 | cysteine |
| CBSL | 274276 | cysteine |
| CCBL1 | 171097 | histidine |
| CCBL2 | 137944 | histidine |
| CCS | 173992 | methionine |
| CEBPA | 245848 | methionine |
| CEBPB | 172216 | methionine |
| CEBPD | 221869 | methionine |
| CEBPE | 092067 | methionine |
| CEBPG | 153879 | methionine |
| CH25H | 138135 | ergosterol |
| COQ6 | 119723 | nicotinic acid |
| CPS1 | 021826 | arginine |
| CTH | 116761 | cysteine |
| CYP51A1 | 001630 | ergosterol |
| DECR1 | 104325 | ergosterol |
| DHFR | 228716 | dIMP |
| DHFRL1 | 178700 | dIMP |
| DHODH | 102967 | uridine |
| DHRS7 | 100612 | lysine |
| DHRS7B | 109016 | lysine |
| DHRS7C | 184544 | lysine |
| DPYD | 188641 | uridine |
| DUT | 128951 | dIMP |
| ETFDH | 171503 | thiamine(1+) |
| FAXDC2 | 170271 | ergosterol |
| FDFT1 | 079459; | ergosterol |
| | 284967 | |
| FDPS | 160752 | ergosterol |
| FDXR | 161513 | uridine |
| FH | 091483 | arginine |
| FPGS | 136877 | methionine |
| G6PD | 160211 | methionine |
| GCAT | 100116 | cysteine |
| GCH1 | 131979 | 5-formyltetrahydrofolicacid |
| GCLC | 001084 | glutathione |
| GFPT1 | 198380 | D-glucosamine |
| GFPT2 | 131459 | D-glucosamine |
| GLRX5 | 182512 | glutamic acid |
| GLUL | 135821 | glutamine |
| GMPS | 163655 | guanine |
| GPT | 167701 | histidine |
| GPT2 | 166123 | histidine |
| GSX2 | 180613 | adenine |
| H6PD | 049239 | methionine |
| HAAO | 162882 | nicotinic acid |
| HLCS | 159267 | biotin |
| HMBS | 256269; | heme |
| | 281702 | |
| HMGCL | 117305 | lysine |
| HMGCLL1 | 146151 | lysine |
| HMGCS1 | 112972 | ergosterol |
| HMGCS2 | 134240 | ergosterol |
| HOXA1 | 105991 | adenine |
| HOXA10 | 253293 | adenine |
| HOXA11 | 005073 | adenine |
| HOXA13 | 106031 | adenine |
| HOXA2 | 105996 | adenine |
| HOXA3 | 105997 | adenine |
| HOXA4 | 197576 | adenine |
| HOXA5 | 106004 | adenine |
| HOXA6 | 106006 | adenine |
| HOXA7 | 122592 | adenine |
| HOXA9 | 078399 | adenine |
| HOXB1 | 120094 | adenine |
| HOXB13 | 159184 | adenine |
| HOXB2 | 173917 | adenine |
| HOXB3 | 120093 | adenine |
| HOXB4 | 182742 | adenine |
| HOXB5 | 120075 | adenine |
| HOXB6 | 108511 | adenine |
| HOXB7 | 260027 | adenine |
| HOXB8 | 120068 | adenine |
| HOXB9 | 170689 | adenine |
| HOXC10 | 180818 | adenine |
| HOXC11 | 123388 | adenine |
| HOXC12 | 123407 | adenine |
| HOXC13 | 123364 | adenine |
| HOXC4 | 198353 | adenine |
| HOXC5 | 172789 | adenine |
| HOXC6 | 197757 | adenine |
| HOXC8 | 037965 | adenine |
| HOXC9 | 180806 | adenine |
| HOXD1 | 128645 | adenine |
| HOXD10 | 128710 | adenine |
| HOXD11 | 128713 | adenine |
| HOXD12 | 170178 | adenine |
| HOXD13 | 128714 | adenine |
| HOXD3 | 128652 | adenine |
| HOXD4 | 170166 | adenine |
| HOXD8 | 175879 | adenine |
| HOXD9 | 128709 | adenine |
| HRSP12 | 132541 | isoleucine |
| HSD11B1 | 117594 | lysine |
| HSD11B1L | 167733 | lysine |
| HSD17B12 | 149084 | ergosterol |
| HSD17B3 | 130948 | ergosterol |
| HSD17B7 | 132196 | ergosterol |
| HSD17B7P2 | 099251 | ergosterol |
| HSDL1 | 103160 | ergosterol |
| HSDL2 | 119471 | ergosterol |
| IBA57 | 181873 | glutamic acid |
| IDO1 | 131203 | nicotinic acid |
| IDO2 | 188676 | nicotinic acid |
| IL4I1 | 104951 | 0.1 mM beta-alanine |
| ILVBL | 105135 | valine, isoleucine |
| IP6K1 | 176095 | arginine |
| IP6K2 | 068745 | arginine |
| IP6K3 | 161896 | arginine |
| IPMK | 151151 | arginine |
| IREB2 | 136381 | leucine |
| ISCA1 | 135070 | lysine |
| ISCA1P1 | 217416 | lysine |
| ISCA2 | 165898 | lysine |
| KATNA1 | 186625 | ethanolamine |
| KATNAL1 | 102781 | ethanolamine |
| KATNAL2 | 167216 | ethanolamine |
| KDM1B | 165097 | 0.1 mM beta-alanine |
| KDSR | 119537 | lysine |
| KMO | 117009 | nicotinic acid |
| KYNU | 115919 | nicotinic acid |
| LGSN | 146166 | glutamine |
| LSS | 281289; | ergosterol |
| | 160285 | |
| MARS | 166986 | methionine |
| MARS2 | 247626 | methionine |
| MAX | 125952 | methionine |
| MITF | 187098 | glutamate(1−) |
| MLX | 108788 | glutamate(1−) |
| MMS19 | 155229 | methionine |
| MPC1 | 060762 | valine, leucine |
| MPC1L | 238205 | valine, leucine |
| MPI | 178802 | D-mannose |
| MSMO1 | 052802 | ergosterol |
| MTHFD1 | 100714 | adenine |
| MTHFD1L | 120254 | adenine |
| MTHFD2 | 065911 | adenine |
| MTHFD2L | 163738 | adenine |
| MTHFR | 177000 | methionine |
| MTRR | 124275 | methionine |
| MVK | 110921 | ergosterol |
| MYB | 118513 | adenine |
| MYBL1 | 185697 | adenine |
| MYBL2 | 101057 | adenine |
| NAGS | 161653 | arginine |
| ODC1 | 115758 | 0.25 mM putrescine |
| OTC | 036473 | arginine |
| PAICS | 128050 | adenine |
| PAOX | 148832 | 0.1 mM beta-alanine |
| PAPSS1 | 138801 | methionine |
| PAPSS2 | 198682 | methionine |
| PDHB | 168291 | tryptophan |
| PDX1 | 139515 | adenine |
| PFAS | 178921 | adenine |
| PIN1 | 127445 | galactose |
| PLCB1 | 182621 | ornithine |
| PLCB2 | 137841 | ornithine |
| PLCB3 | 149782 | ornithine |
| PLCB4 | 101333 | ornithine |
| PLCD1 | 187091 | ornithine |
| PLCD3 | 161714 | ornithine |
| PLCD4 | 115556 | ornithine |
| PLCE1 | 138193 | ornithine |
| PLCG1 | 124181 | ornithine |
| PLCG2 | 197943 | ornithine |
| PLCHI | 114805 | ornithine |
| PLCH2 | 276429; | ornithine |
| | 149527 | |
| PLCL1 | 115896 | ornithine |
| PLCL2 | 154822; | ornithine |
| | 284017 | |
| PLCZ1 | 139151 | ornithine |
| PM20D1 | 162877 | leucine |
| PPAT | 128059 | adenine |
| PSAT1 | 135069 | serine |
| PSPH | 146733 | serine |
| PYCR1 | 183010 | proline |
| PYCR2 | 143811 | proline |
| | 104524 | proline |
| QPRT | 103485 | Nicotinic acid |
| RDH8 | 80511 | Lysine |
| RPUSD2 | 166133 | riboflavin |
| SCD | 99194 | oleic acid |
| SCD5 | 145284 | oleic acid |
| SLC25A19 | 125454 | thiamine |
| SLC25A26 | 144741; | biotin |
| | 282739 | |
| SLC25A34 | 162461 | leucine |
| SLC25A35 | 125434 | leucine |
| SLC7A10 | 130876 | L-arginine |
| SLC7A11 | 151012 | L-arginine |
| SLC7A13 | 164893 | L-arginine |
| SLC7A5 | 103257 | L-arginine |
| SLC7A6 | 103064 | L-arginine |
| SLC7A7 | 155465 | L-arginine |
| SLC7A8 | 092068 | L-arginine |
| SLC7A9 | 021488 | L-arginine |
| SMOX | 088826 | 0.1 mM beta-alanine |
| SMS | 102172 | 0.25 mM spermine |
| SNAPC4 | 165684 | adenine |
| SOD1 | 142168 | methionine |
| SOD3 | 109610 | methionine |
| SQLE | 104549 | ergosterol |
| SRM | 116649 | 0.25 mM spermine |
| TAT | 198650 | histidine |
| TFE3 | 068323 | glutamate(1−) |
| TFEB | 112561 | glutamate(1−) |
| TFEC | 105967 | glutamate(1−) |
| THNSL1 | 185875 | threonine |
| THNSL2 | 144115 | threonine |
| TKT | 163931 | tryptophan |
| TKTL1 | 007350 | tryptophan |
| TKTL2 | 151005 | tryptophan |
| UMPS | 114491 | uridine |
| UROD | 126088 | heme |
| UROS | 188690 | heme |
| USF1 | 158773 | glutamate(1−) |
| USF2 | 105698 | glutamate(1−) |
| VPS33A | 139719 | methionine |
| VPS33B | 184056 | methionine |
| VPS36 | 136100 | ethanolamine |
| VPS4A | 132612 | ethanolamine |
| VPS4B | 119541 | ethanolamine |

CCBL1 may also be referred to as KYAT1. CCBL2 may also be referred to as KYAT3. DHFRL1 may also be referred to as DHFR2. PYCRL may also be referred to as PYCR3. HRSP12 may also be referred to as RIDA.

The auxotrophic factor may be one or two or more nutrients, enzymes, altered pH, altered temperature, non-organic molecules, non-essential amino acids, or altered concentrations of a moiety (compared to normal physiologic concentrations), or combinations thereof. All references to auxotrophic factor herein contemplate administration of multiple factors. Any factor is suitable as long as it is not toxic to the subject and is not bioavailable or present in a sufficient concentration in an untreated subject to sustain growth and reproduction of the modified host cell.

For example, the auxotrophic factor may be a nutrient that is a substance required for proliferation or that functions as a cofactor in metabolism of the modified host cell. Various auxotrophic factors are disclosed in Table 2. In certain embodiments, the auxotrophic factor is selected from biotin, alanine, aspartate, asparagine, glutamate, serine, uridine, valine and cholesterol. Biotin, also known as vitamin B7, is necessary for cell growth. In some instances, valine is needed for the proliferation and maintenance of hematopoietic stem cells. In some instances, the compositions disclosed herein are used to express the enzymes in HSCs that relieve the need for valine supplementation and thereby give those cells a selective advantage when valine is removed from the diet compared to the unmodified cells.

F. Insertion of Constructs

In some embodiments, the auxotrophy-inducing locus is within a target gene selected from those disclosed in Table 2, or the region controlling expression of that gene. In some embodiments, the target gene is selected from UMPS (creating a cell line auxotrophic for uridine) and holocarboxylase synthetase (creating a cell line auxotrophic for biotin). In some embodiments, the auxotrophic factor is selected from biotin, alanine, aspartate, asparagine, glutamate, serine, uridine and cholesterol.

Further disclosed herein are methods of using said nuclease systems to produce the cells described herein, comprising introducing into the cell (a) the components of one or more nuclease systems that target and cleave DNA at an auxotrophy-inducing locus, e.g. meganuclease such as ZFN or TALEN, or CRISPR/Cas nuclease such as CRISPR/Cas9, and (b) a construct or vector as described herein. Each component can be introduced into the cell directly or can be expressed in the cell by introducing a nucleic acid encoding the components of said one or more nuclease systems. The methods can also comprise introducing a second nuclease system, e.g. a second meganuclease or second CRISPR/Cas nuclease that targets and cleaves DNA at a second locus, or a second guide RNA that targets DNA at a second locus, or a nucleic acid that encodes any of the foregoing, and (b) a second construct or vector. The second construct or vector can contain a different transgene, or a second copy of the same transgene, which will then be integrated at the second locus according to such methods described herein.

In certain embodiments, such methods will target integration of the construct containing transgene encoding the therapeutic factor to an auxotrophy-inducing locus in a host cell ex vivo.

Such methods can further comprise (a) introducing a construct or vector into the cell, optionally after expanding said cells, or optionally before expanding said cells, and (b) optionally culturing the cell.

In some embodiments, the disclosure contemplates a method of producing a modified mammalian cell comprising introducing into a mammalian cell: (a) a Cas9 polypeptide, or a nucleic acid encoding said Cas9 polypeptide, (b) a guide RNA specific to an auxotrophy-inducing locus, or a nucleic acid encoding said guide RNA, and (c) a construct or vector as described herein. The methods can also comprise introducing (a) a second guide RNA specific to a second auxotrophy-inducing locus and (b) a second construct or vector. In such methods, the guide RNA can be a chimeric RNA or two hybridized RNAs.

In any of these methods, the nuclease can produce one or more single stranded breaks within the auxotrophy-inducing locus, or a double stranded break within the auxotrophy-inducing locus. In these methods, the auxotrophy-inducing locus is modified by homologous recombination with said construct or vector to result in insertion of the construct into the locus.

The methods can further comprise (c) selecting cells that contain the transgene integrated into the auxotrophy-inducing locus. The selecting steps can include (i) selecting cells that require the auxotrophic factor to survive and optionally (ii) selecting cells that comprise the transgene integrated into the auxotrophy-inducing locus.

In some embodiments, the auxotrophy-inducing locus is a gene encoding uridine monophosphate synthetase (UMPS) and the cells are selected by contacting them with 5-FOA. The UMPS gene is required to metabolize 5-FOA into 5-FUMP, which is toxic to cells due to its incorporation into RNA/DNA. Thus, cells which have a disruption in the UMPS gene will survive 5-FOA treatment. The resulting cells will all be auxotrophic, although not all cells may contain the transgene. Subsequent positive selection for the transgene will isolate only modified host cells that are auxotrophic and that are also capable of expressing the transgene.

In some embodiments, the present disclosure provides a method of creating a modified human host cell comprising the steps of: (a) obtaining a pool of cells, (b) using a nuclease to introduce a construct to the auxotrophy-inducing locus, for example by knocking out or downregulating expression of a gene, and (c) screening for auxotrophy, and (d) screening for the presence of the transgene.

The screening step may be carried out by culturing the cells with or without one of the auxotrophic factors disclosed in Table 2.

Techniques for insertion of constructs comprising transgenes, including large transgenes, capable of expressing functional or therapeutic factors, antibodies, and cell surface receptors are known in the art (See, e.g. Bak and Porteus, Cell Rep. 2017 Jul. 18; 20(3): 750-756 (integration of EGFR); Kanojia et al., Stem Cells. 2015 October; 33(10):2985-94 (expression of anti-Her2 antibody); Eyquem et al., Nature. 2017 Mar. 2; 543(7643):113-117 (site-specific integration of a CAR); O'Connell et al., 2010 PLoS ONE 5(8): e12009 (expression of human IL-7); Tuszynski et al., Nat Med. 2005 May; 11(5):551-5 (expression of NGF in fibroblasts); Sessa et al., Lancet. 2016 Jul. 30; 388(10043):476-87 (expression of arylsulfatase A in ex vivo gene therapy to treat MLD); Rocca et al., Science Translational Medicine 25 Oct. 2017: Vol. 9, Issue 413, eaaj2347 (expression of frataxin); Bak and Porteus, Cell Reports, Vol. 20, Issue 3, 18 Jul. 2017, Pages 750-756 (integrating large transgene cassettes into a single locus), Dever et al., Nature 17 Nov. 2016: 539, 384-389 (adding tNGFR into hematopoietic stem cells (HSC) and HSPCs to select and enrich for modified cells); each of which is hereby incorporated by reference in its entirety.

G. Controlling Gene Expression

In some instances, the transgene is optionally linked to one or more expression control sequences, including the gene's endogenous promoter, or heterologous constitutive or inducible promoters, enhancers, tissue-specific promoters, or post-transcriptional regulatory sequences. For example, one can use tissue-specific promoters (transcriptional targeting) to drive transgene expression or one can include regulatory sequences (microRNA (miRNA) target sites) in the RNA to avoid expression in certain tissues (post-transcriptional targeting). In some instances, the expression control sequence functions to express the therapeutic transgene following the same expression pattern as in normal individuals (physiological expression) (See Toscano et al., Gene Therapy (2011) 18, 117-127 (2011), incorporated herein by reference in its entirety for its references to promoters and regulatory sequences).

Constitutive mammalian promoters include, but are not limited to, the promoters for the following genes: hypoxanthine phosphoribosyl transferase (HPTR), adenosine deaminase, pyruvate kinase, a-actin promoter and other constitutive promoters. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Commonly used promoters including the CMV (cytomegalovirus) promoter/enhancer, EF1a (elongation factor 1a), SV40 (simian virus 40), chicken β-actin and CAG (CMV, chicken β-actin, rabbit β-globin), Ubiquitin C and PGK, all of which provide constitutively active, high-level gene expression in most cell types. Other constitutive promoters are known to those of ordinary skill in the art.

Inducible promoters are activated in the presence of an inducing agent. For example, the metallothionein promoter is activated to increase transcription and translation in the presence of certain metal ions. Other inducible promoters include alcohol-regulated, tetracycline-regulated, steroid-regulated, metal-regulated, nutrient-regulated promoters, and temperature-regulated promoters.

In certain embodiments, the promoter is tissue-specific. For example, the promoter may be activated by differentiation of the cell into the associated tissue. For liver-specific targeting, natural and chimeric promoters and enhancers have been incorporated into viral and non-viral vectors to target expression of factor VIIa, factor VIII or factor IX to hepatocytes. Promoter regions from liver-specific genes such as albumin and human al antitrypsin (hAAT) are good examples of natural promoters. Alternatively, chimeric promoters have been developed to increase specificity and/or vectors efficiency. Good examples are the (ApoE)4/hAAT chimeric promoter/enhancer, harboring four copies of a liver-specific ApoE/hAAT enhancer/promoter combination and the DC172 chimeric promoter, consisting in one copy the hAAT promoter and two copies of the α(1)-microglobulin enhancer.

For muscle-specific targeting, natural (creatine kinase promoter-MCK, desmin) and synthetic (α-myosin heavy chain enhancer-/MCK enhancer-promoter (MHCK7)) promoters have been included in viral and non-viral vectors to achieve efficient and specific muscle expression.

For endothelium-specific targeting, both natural (vWF, FLT-1 and ICAM-2) and synthetic promoters have been used to drive endothelium-specific expression.

For myeloid cell targeting, a synthetic chimeric promoter that contains binding sites for myeloid transcription factors CAAT box enhancer-binding family proteins (C/EBPs) and PU.1, which are highly expressed during granulocytic differentiation, has been reported to direct transgene expression primarily in myeloid cells (See, Santilli et al., Mol Ther. 2011 January; 19(1):122-32, which is hereby incorporated by reference in its entirety. CD68 may also be used for myeloid targeting.

Examples of tissue-specific promoters and vectors for gene therapy of genetic diseases are shown in Table 3.

TABLE 3
Tissue-specific promoters

| Promoter | Vector type | Target cell/tissue |
|---|---|---|
| WAS proximal promoter | HIV-1-based vectors | Hematopoietic cells |
| CD4 mini-promoter/enhancer | MLV-based vectors | T cells |
| CD2 locus control region | MLV based and HIV-1-based | T cells |
| | vectors | |
| CD4 minimal promoter and proximal enhancer and silencer | HIV-1-based vectors | T cells |
| CD4 mini-promoter/enhancer | HIV-1-based vectors | T cells |
| GATA-1 enhancer HS2 within the LTR | SFCM retroviral vector | Erythroid linage |
| Ankyrin-1 and α-spectrin promoters combined or not with HS- | HIV-1-based vectors | Erythroid linage |
| 40, GATA-1, ARE and intron 8 enhancers | | |
| Ankyrin-1 promoter/β-globin HS-40 enhancer | HIV-1-based vectors | Erythroid linage |
| GATA-1 enhancer HS1 to HS2 within the retroviral LTR | SFCM retroviral vector | Erythroid linage |
| Hybrid cytomegalovirus (CMV) enhancer/β-actin promoter | Sleeping Beauty transposon | Erythroid linage |
| MCH II-specific HLA-DR promoter | HIV-1-based vectors | APCs |
| Fascin promoter (pFascin) | Plasmid | APCs |
| Dectin-2 gene promoter | HIV-1-based vectors | APCs |
| 5′ untranslated region from the DC-STAMP | HIV-1-based vectors | APCs |
| Heavy chain intronic enhancer (Eμ) and matrix attachment | HIV-1-based vectors | B cells |
| regions | | |
| CD19 promoter | HLV based vectors | B cells |
| Hybrid immunoglobulin promoter (Igk promoter, intronic | HIV-1-based vectors | B cells |
| Enhancer and 3′ enhancer from Ig genes) | | |
| CD68L promoter and first intron | MLV-based vectors | Megakaryocytes |
| Glycoprotein Ibα promoter | HIV-1-based vectors | Megakaryocytes |
| Apolipoprotein E (Apo E) enhancer/alpha1-antitrypsin (hAAT) | HLV based vectors | Hepatocytes |
| promoter (ApoE/hAAT) | | |
| HAAT promoter/Apo E locus control region | Plasmid | Hepatocytes |
| Albumin promoter | HIV-1-based vectors | Hepatocytes |
| HAAT promoter/four copies of the Apo E enhancer | AAV-2-based vectors | Hepatocytes |
| Albumin and hAAT promoters/α1-microglobulin and | Plasmid | Hepatocytes |
| prothrombin enhancers | | |
| HAAT promoter/Apo E locus control region | AAV8 | Hepatocytes |
| hAAT promoter/four copies of the Apo E enhancer | AAV2/8 | Hepatocytes |
| TBG promoter (thyroid hormone-binding globulin promoter | AAV | Hepatocytes |
| and α1-microglobulin/bikunin enhancer) | | |
| DC172 promoter (α1-antitrypsin promoter and α1- | Adenovirus, plasmid | Hepatocytes |
| microglobulin enhancer) | | |
| LCAT, kLSP-IVS, ApoE/hAAT and liver-fatty acid-binding | AAV1, AAV2, AAV6, AAV8 | Hepatocytes |
| protein promoters | | |
| RU486-responsive promoter | Adenovirus | Hepatocytes |
| Creatine kinase promoter | Adenovirus | Muscle |
| Creatine kinase promoter | AAV6 | Muscle |
| Synthetic muscle-specific promoter C5-12 | AV-1 | Muscle |
| Creatine kinase promoter | AAV2/6 | Muscle |
| Hybrid enhancer/promoter regions of α-myosin and creatine | AAV6 | Muscle |
| kinase (MHCK7) | | |
| Hybrid enhancer/promoter regions of α-myosin and creatine | AAV2/8 | Muscle |
| kinase | | |
| Synthetic muscle-specific promoter C5-12 | HIV-1-based vectors | Muscle |
| Cardiac troponin-I proximal promoter | HIV-1-based vectors | Cardiomyocytes |
| E-selectin and KDR promoters | MLV-based vectors | Endothelial cell |
| Prepro-endothelin-1 promoter | MLV-based vectors | Endothelial cell |
| KDR promoter/hypoxia-responsive element | MLV-based vectors | Endothelial cell |
| Flt-1 promoter | Adenovirus | Endothelial cell |
| Flt-1 promoter | Adenovirus | Endothelial cell |
| ICAM-2 promoter | Plasmid | Endothelial cell |
| Synthetic endothelial promoter | HIV-1-based vectors | Endothelial cell |
| Endothelin-1 gene promoter | Sleeping Beauty transposon | Endothelial cell |
| Amylase promoter | Adenovirus | Pancreas |
| Insulin and human pdx-1 promoters | Adenovirus | Pancreas |
| TRE-regulated insulin promoter | Plasmid | Pancreas |
| Enolase promoter | Herpesvirus | Neurons |
| Enolase promoter | Adenoviruses | Neurons |
| TRE-regulated synapsin promoter | Adenoviruses | Neurons |
| Synapsin 1 promoter | Adenoviruses | Neurons |
| PDGF-β promoter/CMV enhancer | Plasmid | Neurons |
| PDGF-β, synapsin, tubulin-α and Ca2+/calmodulin-PK2 | HIV-1-based vectors | Neurons |
| promoters combined with CMV enhancer | | |
| Phosphate-activated glutaminase and vesicular glutamate | Herpesvirus | Glutamatergic |
| transporter-1 promoters | | neurons |
| Glutamic acid decarboxylase-67 promoter | Herpesvirus | GABAergic neuron |
| Tyrosine hydroxylase promoter | Herpesvirus | Catecholaminergic |
| | | neurons |
| Neurofilament heavy gene promoter | Herpesvirus | Neurons |
| Human red opsin promoter | Recombinant AAV | Cone cells |
| Keratin-18 promoter | Adenovirus | Epithelial cells |
| keratin-14 (K14) promoter | Lentiviral vectors | Epithelial cells |
| Keratin-5 promoter | HIV-1-based vectors | Epithelial cells |

In some embodiments, the promoters for use in regulating transgene expression of the constructs described herein include promoters that are specific for T reg-like cells. Expression profiles of stable T reg cell populations have been described by Passerini et al, who have shown that conventional CD4+ T cells can be converted into fully functional T reg-like cells by introducing FOXP3 expression. (See Passerini, Laura, et al. “CD4+ T cells from IPEX patients convert into functional and stable regulatory T cells by FOXP3 gene transfer.” Science translational medicine 5.215 (2013): 215ra174-215ra174, the disclosure of which is incorporated by reference herein in its entirety.) Thus, in some embodiments, a construct as described herein can be regulated by an expression control sequence of FOXP3 (ENSG00000049768), for example using the FOXP3 promoter.

In some embodiments, the promoters for use in regulating transgene expression of the constructs described herein include promoters that are specific for a cell naïveté-associated promoter (e.g., CD45RA/RO [ENSG00000081237] or CCR7 [ENSG00000126353]). Thus, in some embodiments, a construct as described herein can be regulated by an expression control sequence or promoter of a CD45 receptor A or O, or CCR7.

Examples of physiologically regulated promoters and vectors for gene therapy of genetic diseases are shown in Table 4.

TABLE 4
Physiologically regulated vectors

| Promoter | Vector type | Target cell/tissue |
|---|---|---|
| WAS proximal promoter (1600 bp) | HIV-1-based vectors | Hematopoietic cells |
| WAS proximal promoter (500 bp) | HIV-1-based vectors | Hematopoietic cells |
| WAS proximal promoter (170 bp) | HIV-1-based vectors | Hematopoietic cells |
| WAS proximal promoter (500 bp)/WAS | HIV-1-based vectors | Hematopoietic cells |
| alternative promoter (386 bp) | | |
| CD40L promoter and regulatory sequences | Human artificial | Activated T cells |
| | chromosome (HAC) | |
| CD40L promoter | HIV-1-based vectors | Activated T cells |
| β-Globin promoter/LCR | HIV-1-based vectors | Erythroid linage |
| β-Globin and θ-globin promoters combined or | HIV-1-based vectors | Erythroid linage |
| not with HS-40, GATA-1, ARE, and intron 8 | | |
| enhancers | | |
| β-Globin, LCR HS4, HS3, HS2 and a truncated | HIV-1-based vectors | Erythroid linage |
| β-globin intron 2 | | |
| β-Globin promoter/LCR/cHS4 | HIV-1-based vectors | Erythroid linage |
| HSFE/LCR/β-globin promoter | MSCV retroviral vector | Erythroid linage |
| Integrin αIIb promoter (nucleotides −889 to +35) | MLV-based vectors | Megakaryocytes |
| Dystrophin promoter and regulatory sequences | HAC | Muscle |
| Endoglin promoter | Plasmid | Endothelial cells |
| RPE65 promoter | AAV2/4 | Retinal pigmented epithelium |
| TRE-regulated synapsin promoter | Adenoviruses | Neurons |

Tissue-specific and/or physiologically regulated expression can also be pursued by modifying mRNA stability and/or translation efficiency (post-transcriptional targeting) of the transgenes. Alternatively, the incorporation of miRNA target recognition sites (miRTs) into the expressed mRNA has been used to recruit the endogenous host cell machinery to block transgene expression (detargeting) in specific tissues or cell types. miRNAs are noncoding RNAs, approximately 22 nucleotides, that are fully or partially complementary to the 3′ UTR region of particular mRNA, referred to as miRTs. Binding of a miRNA to its particular miRTs promotes translational attenuation/inactivation and/or degradation. Regulation of expression through miRNAs is described in Geisler and Fechner, World J Exp Med. 2016 May 20, 6(2): 37-54; Brown and Naldini, Nat Rev Genet. 2009 August, 10(8):578-85; Gentner and Naldini, Tissue Antigens. 2012 November, 80(5):393-403; each of which is hereby incorporated by reference in its entirety. Engineering miRTs-vector recognized by a specific miRNA cell type has been shown to be an effective way for knocking down the expression of a therapeutic gene in undesired cell types (See, Toscano et al., supra, which is hereby incorporated by reference in its entirety).

The transgene expressing the knocked-out auxotrophy-inducing gene, thereby rescuing auxotrophy upon cell differentiation or plasmid transduction, can be tagged with a conditional destabilization domain. A destabilization domain as used herein refers to a peptide, protein, or fraction thereof which confers a destabilizing property to a gene product with which it is associated. Destabilization domains are known (see, for example, WO 2018/160993, the disclosure of which is incorporated by reference herein in its entirety). As described in WO 2018/160993, conditional destabilization domains can be activated to induce stability or instability of the gene product with which it is associated based on the presence or absence of a stimulus or ligand. In the present context, a destabilization domain can be genetically appended to, for example, a re-expressed auxotrophy-inducing gene such that, upon integration and expression of the transgene providing the re-expressed gene, a destabilization domain is attached to the gene. The gene and destabilization domain combination would remain “stable” during the in vitro selection process, where progenitor or untransduced cells are removed, by, for example, providing the ligand that confers a stability signal to the destabilization domain. The combination could then be made unstable by removing the ligand on or before introduction into the patient, thereby making the cells auxotrophic again. This provides the added benefit of an additional functional safety switch, whereby, the cells generated using auxotrophic selection methods described herein can be made, in some cases, to conditionally destabilize the re-expressed auxotrophy-inducing gene and in other cases to stabilize the re-expressed auxotrophy-inducing gene. Ribozymes, self-cleaving ribonucleotide elements, can be used to similar effect by encoding for ribozymes to trigger self-destruction of RNA transcripts of the transgene encoding the auxotrophy-inducing gene. Therefore, any of the auxotrophy-inducing genes described herein can be made conditional by at least one of a destabilization domain or a conditional ribozyme switch.

### III. Methods of the Present Disclosure

### A. Single Auxotrophic Systems

The present disclosure provides methods of using the constructs described herein to generate populations of differentiated cells. The methods provided can be used to generate pure and/or enriched populations of particular cell types. Generating pure and enriched populations of particular cell types can be useful in therapeutic and diagnostic applications. For example, purified or enriched populations of glucose-responsive mature beta cells derived from differentiated progenitor cells can be useful in the treatment of diabetes.

The differentiated cells produced by the methods described can be derived from progenitor cells. In some embodiments, the progenitor cells can be induced pluripotent stem cells (iPSCs), hematopoietic stem cells (HSCs), embryonic stem cells, transdifferentiated stem cells, neural progenitor cells, mesenchymal stem cells, osteoblasts, and cardiomyocytes.

In some embodiments, the methods comprise contacting a plurality of progenitor cells with a nuclease system to induce recombination or homologous recombination in the cells. In some embodiments, CRISPR/Cas is the nuclease system deployed to induce homologous recombination. The CRISPR/Cas system can comprise a guide RNA (gRNA) targeting an inessential portion of a promoter of an auxotrophy-inducing gene. As used herein, an “inessential portion” refers to a portion of a promoter of a gene which, when disrupted by a nuclease and/or when interrupted by a transgene insertion, the promoter remains functional and responsive to endogenous cellular stimuli, including transcription and other factors. Once the nuclease system has targeted the inessential portion of the promoter of an auxotrophy-inducing gene, a construct can be inserted to induce homologous recombination at the site targeted by the nuclease such that the construct is inserted into the genome of the cell. The construct can be inserted biallelically, resulting in homozygous knock-in cells. Auxotrophy-inducing loci that can be targeted for homologous recombination are provided in Table 2. The construct inserted (e.g., biallelically) can comprise all or a portion of a tissue-specific promoter and at least a portion of the gene selected from the group consisting of: AACS, AADAT, AASDHPPT, AASS, ACAT1, ACCS, ACCSL, ACO1, ACO2, ACSS3, ADSL, ADSS, ADSSL1, ALAD, ALAS1, ALAS2, ALDH1A1, ALDH1A2, ALDH1A3, ALDH1B1, ALDH2, AMD1, ASL, ASS1, ATF4, ATF5, AZIN1, AZIN2, BCAT1, BCAT2, CAD, CBS, CBSL, CCBL1, CCBL2, CCS, CEBPA, CEBPB, CEBPD, CEBPE, CEBPG, CH25H, COQ6, CPS1, CTH, CYP51A1, DECR1, DHFR, DHFRL1, DHODH, DHRS7, DHRS7B, DHRS7C, DPYD, DUT, ETFDH, FAXDC2, FDFT1, FDPS, FDXR, FH, FPGS, G6PD, GCAT, GCH1, GCLC, GFPT1, GFPT2, GLRX5, GLUL, GMPS, GPT, GPT2, GSX2, H6PD, HAAO, HLCS, HMBS, HMGCL, HMGCLL1, HMGCS1, HMGCS2, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HRSP12, HSD11B1, HSD11B1L, HSD17B12, HSD17B3, HSD17B7, HSD17B7P2, HSDL1, HSDL2, IBA57, IDO1, IDO2, IL4I1, ILVBL, IP6K1, IP6K2, IP6K3, IPMK, IREB2, ISCA1, ISCA1P1, ISCA2, KATNA1, KATNAL1, KATNAL2, KDM1B, KDSR, KMO, KYNU, LGSN, LSS, MARS, MARS2, MAX, MITF, MLX, MMS19, MPC1, MPC1L, MPI, MSMO1, MTHFD1, MTHFD1L, MTHFD2, MTHFD2L, MTHFR, MTRR, MVK, MYB, MYBL1, MYBL2, NAGS, ODC1, OTC, PAICS, PAOX, PAPSS1, PAPSS2, PDHB, PDX1, PFAS, PIN1, PLCB1, PLCB2, PLCB3, PLCB4, PLCD1, PLCD3, PLCD4, PLCE1, PLCG1, PLCG2, PLCH1, PLCH2, PLCL1, PLCL2, PLCZ1, PM20D1, PPAT, PSAT1, PSPH, PYCR1, PYCR2, QPRT, RDH8, RPUSD2, SCD, SCD5, SLC25A19, SLC25A26, SLC25A34, SLC25A35, SLC7A10, SLC7A11, SLC7A13, SLC7A5, SLC7A6, SLC7A7, SLC7A8, SLC7A9, SMOX, SMS, SNAPC4, SOD1, SOD3, SQLE, SRM, TAT, TFE3, TFEB, TFEC, THNSL1, THNSL2, TKT, TKTL1, TKTL2, UMPS, UROD, UROS, USF1, USF2, VPS33A, VPS33B, VPS36, VPS4A, and VPS4B. Cells produced in this way will be auxotrophic for an auxotrophic factor corresponding to the auxotrophy-inducing locus.

The cells can be propagated by contacting them with the auxotrophic factor. Only those cells having the transgene construct expressing the auxotrophy-inducing gene or portion thereof will survive withdrawal of the auxotrophic factor. Cells in the population not rendered auxotrophic due to failure of the nuclease and/or recombination steps will survive in some embodiments. In embodiments where the auxotrophy-inducing locus is a gene encoding uridine monophosphate synthetase (UMPS), the cells can be selected for by contacting them with 5-FOA. The UMPS gene is required to metabolize 5-FOA into 5-FUMP, which is toxic to cells due to its incorporation into RNA/DNA. Thus, cells which have a disruption in the UMPS gene will survive 5-FOA treatment. The resulting cells will all be auxotrophic, although not all cells will contain the transgene. Subsequent positive selection for the transgene will isolate only modified host cells that are auxotrophic and that are also capable of expressing the transgene.

The methods described herein can be used for stimulating differentiation of progenitor cells into a tissue associated with a tissue-specific promoter. In this context, the transgene construct re-expressing the auxotrophy-inducing gene can be regulated by endogenous tissue-specific factors that are specifically expressed in the desired differentiated cell or tissue type. Thus, in some embodiments, the constructs described herein are expressed in response to differentiation of a cell to the desired cell fate, cell type, or tissue type. In this way, the methods can be used to select for populations of, for example, in vitro differentiated cells which have differentiated to the desired cell type. The methods of using the constructs described herein to generate populations of differentiated cells can further comprise removing the auxotrophic factor, thereby selecting for differentiated cells.

In some embodiments, the tissue-specific promoter of the transgene replaces the promoter for the UMPS gene or other auxotrophy-inducing gene target.

In some embodiments, the construct inserted with the transgene can further comprise a therapeutic factor or a gene encoding a therapeutic factor. The therapeutic factor can be expressed as a cassette with targeted auxotrophy-inducing gene or portion thereof. Expression of the construct, including the therapeutic factor, can be optimized by creating a polycistronic construct having, for example, a linker between two or more expressed components, wherein the linker is an internal ribosome entry site (IRES) or a peptide 2A sequence (P2A) or the like. Exemplary linker sequences are provided as SEQ ID NOs: 20, 22, 24, 25, 26, 27, 28, 29, and 30.

Expression of the re-expressed auxotrophy-inducing gene or other transgene in some embodiments can be regulated by a eukaryotic promoter sequence such as EF1a (SEQ ID NO: 31 or SEQ ID NO: 32). Bicistronic or multicistronic constructs can be prepared by separating the expressed components of the construct with linkers as described or using an internal ribosome entry site (IRES) such as that of SEQ ID NO: 33.

Termination and polyadenylation signal sequences can be used to terminate and stabilize the transcript produced from the transgene constructs described herein. In some embodiments, transcription is terminated and stabilized using a bovine growth hormone (bGH) poly-adenylation signal sequence, such as that of SEQ ID NO: 39 or 40.

To direct homologous recombination at the targeted auxotrophy-inducing gene locus, the portion of the construct including nucleotide sequence of the auxotrophy-inducing gene locus can serve as a homology arm that is complimentary to the endogenous sequence, such that it will hybridize and initiate homologous recombination. In some embodiments, directed homologous recombination at the targeted auxotrophy-inducing gene locus entails inserting the construct into the auxotrophy-inducing gene locus such that expression of the gene is not disrupted. For example, the homologous recombination construct targeting an inessential portion of a promoter of an auxotrophy-inducing gene can be inserted in-frame with the auxotrophy-inducing gene, resulting in insertion of the construct including, e.g., a tissue-specific promoter, that leaves intact the open reading frame of the auxotrophy-inducing gene.

The methods described herein can be used to select for cells that have differentiated into a particular tissue. The tissue can be one selected from the group consisting of: adipose tissue, adrenal gland, ascites, bladder, blood, bone, bone marrow, brain, cervix, connective tissue, ear, embryonic tissue, esophagus, eye, heart, hematopoietic tissue, intestine, kidney, larynx, liver, lung, lymph, lymph node, mammary gland, mouth, muscle, nerve, ovary, pancreas, parathyroid, pharynx, pituitary gland, placenta, prostate, salivary gland, skin, spleen, stomach, testis, thymus, thyroid, tonsil, trachea, umbilical cord, uterus, endocrine, neuronal tissue, and vascular tissue. In some embodiments, the differentiated cell is an immune cell, and the immune cell can be differentiated into, for example, a T cell, a B cell, or a natural killer (NK) cell.

Tissue-specific promoters that can be utilized in the constructs and methods described herein can be selected from the group consisting of: WAS proximal promoter; CD4 mini-promoter/enhancer; CD2 locus control region; CD4 minimal promoter and proximal enhancer and silencer; CD4 mini-promoter/enhancer; GATA-1 enhancer HS2 within the LTR; Ankyrin-1 and α-spectrin promoters combined or not with HS-40, GATA-1, ARE and intron 8 enhancers; Ankyrin-1 promoter/β-globin HS-40 enhancer; GATA-1 enhancer HS1 to HS2 within the retroviral LTR; Hybrid cytomegalovirus (CMV) enhancer/β-actin promoter; MCH II-specific HLA-DR promoter; Fascin promoter (pFascin); Dectin-2 gene promoter; 5′ untranslated region from the DC-STAMP; Heavy chain intronic enhancer (Ep) and matrix attachment regions; CD19 promoter; Hybrid immunoglobulin promoter (Igk promoter, intronic Enhancer and 3′ enhancer from Ig genes); CD68L promoter and first intron; Glycoprotein Ibα promoter; Apolipoprotein E (Apo E) enhancer/alpha1-antitrypsin (hAAT) promoter (ApoE/hAAT); HAAT promoter/Apo E locus control region; Albumin promoter; HAAT promoter/four copies of the Apo E enhancer; Albumin and hAAT promoters/al-microglobulin and prothrombin enhancers; HAAT promoter/Apo E locus control region; hAAT promoter/four copies of the Apo E enhancer; TBG promoter (thyroid hormone-binding globulin promoter and α1-microglobulin/bikunin enhancer); DC172 promoter (α1-antitrypsin promoter and α1-microglobulin enhancer); LCAT, kLSP-IVS, ApoE/hAAT and liver-fatty acid-binding protein promoters; RU486-responsive promoter; Creatine kinase promoter; Creatine kinase promoter; Synthetic muscle-specific promoter C5-12; Creatine kinase promoter; Hybrid enhancer/promoter regions of α-myosin and creatine kinase (MHCK7); Hybrid enhancer/promoter regions of α-myosin and creatine kinase; Synthetic muscle-specific promoter C5-12; Cardiac troponin-1 proximal promoter; E-selectin and KDR promoters; Prepro-endothelin-1 promoter; KDR promoter/hypoxia-responsive element; Flt-1 promoter; Flt-1 promoter; ICAM-2 promoter; Synthetic endothelial promoter; Endothelin-1 gene promoter; Amylase promoter; Insulin and human pdx-1 promoters; TRE-regulated insulin promoter; Enolase promoter; Enolase promoter; TRE-regulated synapsin promoter; Synapsin 1 promoter; PDGF-β promoter/CMV enhancer; PDGF-β, synapsin, tubulin-α and Ca2+/calmodulin-PK2 promoters combined with CMV enhancer; Phosphate-activated glutaminase and vesicular glutamate transporter-1 promoters; Glutamic acid decarboxylase-67 promoter; Tyrosine hydroxylase promoter; Neurofilament heavy gene promoter; Human red opsin promoter; Keratin-18 promoter; keratin-14 (K14) promoter; and Keratin-5 promoter.

In some embodiments, the constructs described herein tag an expressed gene product with a conditional destabilization domain or insert a ribozyme switch in the transcribed message of the construct, leading to conditional destabilization of the gene product or destruction of the RNA message.

Some embodiments of the methods of selecting for populations of differentiated cells described herein can comprise contacting progenitor cells with a construct designed to knock-in a DNA sequence encoding one or more progenitor cell-specific miRNA target sites into a an auxotrophy-inducing gene. The miRNA target sites thus knocked-into the auxotrophy-inducing gene result in the progenitor cells being auxotrophic for an auxotrophic factor corresponding to the auxotrophy-inducing gene (see Table 2, for example). Differentiation of the progenitor cells into a non-progenitor cell fate results in the one or more progenitor cell-specific miRNAs no longer being expressed, thereby relieving the miRNA-mediated suppression of the auxotrophy-inducing gene and enabling survival of the cells upon withdrawal of the auxotrophic factor. Differentiated cell populations selected for using the methods described herein can be purified or enriched populations of the desired cell type. The differentiated cells can be administered to subjects in need of the cell type to treat a disease or condition. For instance, differentiated immune cells as described can be administered to treat patients in need of immunotherapy, or differentiated mature beta cells as described can be administered to treat patients having insulin disorders. Thus, provided herein are methods of providing a plurality of auxotrophic progenitor cells which have been generated by knockout of the auxotrophy-inducing gene; and inserting a construct comprising an open reading frame of the gene into a tissue-specific gene locus, wherein expression of the tissue-specific gene is not disrupted, thereby producing the auxotrophic factor or re-expressed auxotrophy-inducing gene upon differentiation of the progenitor cells into the tissue associated with the tissue-specific gene locus. The progenitor cells can be, for example, iPSCs or embryonic stem cells.

In some embodiments, the auxotrophy-inducing gene or other gene construct introduced into the cell can be via plasmid integration or via episomal expression. Introduction of the constructs described herein into the cells for selective propagation and differentiation can be achieved using, for example, a DNA plasmid, an adeno-associated virus (AAV) vector, or a nanoparticle delivery system.

B. Split Auxotrophic Systems

Cells and cell populations made auxotrophic using the methods described herein can be maintained (i.e., sustained in a viable and/or proliferative state) in vivo or in vitro by at least two distinct methods: 1) by providing the auxotrophic factor to the cells; or 2) by rescuing the auxotrophy by expressing in the cells the knocked out or downregulated auxotrophic gene. For example, in the case of UMPS^{-/-} cells as described herein, the cells can be maintained by providing uridine or by expressing an UMPS transgene (i.e., UMPS re-expression). As described herein, re-expression of the auxotrophic gene allows for selection of successfully transfected cells in a population of cells when the auxotrophic factor is removed or withdrawn. Placing re-expression of the auxotrophic gene under control of an expression control sequence comprising, e.g., a tissue-specific promoter as described herein, enables selection of successfully transfected cells and further enables selection of the desired differentiated cell population (i.e., cells expressing the factor(s) specific for the selected tissue-specific promoter(s)). In some embodiments of the methods described herein, the desired differentiated cells are indicated by their expression of at least one tissue-specific factor. In some embodiments, the desired differentiated cells are indicated by their expression of two or more tissue-specific factors. In some embodiments, the desired differentiated cells are indicated by their expression of three or more tissue-specific factors. In some embodiments, the desired differentiated cells are indicated by their expression of four or more tissue-specific factors. In some embodiments, the desired differentiated cells are indicated by their expression of five or more tissue-specific factors. In some embodiments, the desired differentiated cells are indicated by their expression of six or more tissue-specific factors. The specificity of selection for the desired differentiated cells can be increased by selecting for more than one tissue-specific factor. That is, selecting from a population of cells only those cells expressing two or more, three or more, four or more, five or more, or six or more tissue-specific factors indicative of the desired differentiated cell population improves the specificity of the selection method and increases the purity of the selected-for population of desired differentiated cells.

In some embodiments, selecting from a population of cells only those cells expressing two or more, three or more, four or more, five or more, or six or more tissue-specific factors indicative of the desired differentiated cell population comprises delivering auxotrophic genes to (i.e., re-expressing auxotrophic genes at) two or more auxotrophy-inducing loci, three or more auxotrophy-inducing loci, four or more auxotrophy-inducing loci, five or more auxotrophy-inducing loci, or six or more auxotrophy-inducing loci.

In some embodiments, it is unfavorable or undesirable to replace or disrupt more than one auxotrophy-inducing locus with more than one re-expressed auxotrophic gene, yet it is still desirable to select for more than one tissue-specific factor indicative of the desired differentiated cell population. Under these circumstances, auxotrophic genes having more than one independent functional domains or subunits can be exploited to introduce “split auxotrophy” and enable selection for more than one tissue-specific factor indicative of the desired differentiated cell population. For instance, an auxotrophic gene can have a first independent functional domain and a second independent functional domain. Re-expression of the auxotrophic gene can be achieved by expressing the auxotrophic gene as a whole functional gene or can be achieved by splitting the expression of the first and second independent functional domains with the first independent functional domain under control of a first expression control sequence and the second independent functional domain under control of a second expression control sequence. The first independent functional domain can be delivered to a first locus. In some embodiments, the second independent functional domain can be delivered to a second locus. The first locus can be the auxotrophy-inducing locus. The second locus can be, for example, a safe harbor locus such as CCR5. In some embodiments, the CCR5 locus is targeted using CCR5 homology arms, wherein the homology arms are defined as a left and a right homology arm. An exemplary CCR5 left homology arm is defined as SEQ ID NO: 11. Alternative CCR5 left homology arms are provided as SEQ ID NO: 13 and SEQ ID NO: 14. An exemplary CCR5 right homology arm is defined as SEQ ID NO: 12. An alternative CCR5 right homology arm is provided as SEQ ID NO: 15 Alternatively, both the first and second independent functional domains can be delivered to a safe harbor locus such as CCR5, for example, using CCR5 left and right homology arms of SEQ ID NO: 11 and SEQ ID NO: 12, respectively.

Left and right homology arms for CCR5 should have homology to the target CCR5 locus of at least 200 bp but ideally 400 bp on each side (left and right) to assure high levels of reproducible targeting to the locus. The CCR5 left and right homology arms described herein (i.e., SEQ ID NO: 11, 12, 13, 14, and 15) are provided as examples only. Effective homology arms can be designed to target the CCR5 locus using about 100, about 200, about 300, about 400, about 500, or about 600 nucleotides targeting the left (5′) side of the construct to a position in the target locus, and about 100, about 200, about 300, about 400, about 500, or about 600 nucleotides targeting the right (3′) side of the construct to a position in the target locus. Any other non-CCR5 genetic locus can be targeted for homologous recombination in similar fashion.

The first expression control sequence can be a first tissue-specific promoter regulated by, e.g., a first transcription factor specifically expressed in the desired differentiated cell population. Likewise, the second expression control sequence can be a second tissue-specific promoter regulated by, e.g., a second transcription factor specifically expressed in the desired differentiated cell population. In this manner, multiple tissue-specific factors can be selected for to improve the specificity of the desired differentiated cell population without the need to knockout or downregulate more than a single auxotrophy-inducing gene. Thus, the auxotrophy of the engineered cells is said to be “split,” requiring re-expression of each of the auxotrophic gene's independent functional domains in order to survive removal or withdrawal of the auxotrophic factor. This permits the use of one auxotrophic factor to select for multiple transgene integrations.

In some embodiments, the auxotrophy-inducing gene is human UMPS (ENSG00000114491) and the first independent functional domain comprises orotate phosphoribosyltransferase (orotic acid phosphoribosyltransferase or OPRT) and the second independent functional domain comprises orotidine 5′-phosphate decarboxylase (OMPdecase or ODC). In human UMPS, OPRT and ODC comprise separate independent functional domains within the same gene, whereas the two domains are expressed by separate genes in other organisms. Thus, in human UMPS^{-/-} cells, UMPS activity can be replaced by re-expression of UMPS cDNA (using, for example, the nucleotide sequence of SEQ ID NO: 1 or SEQ ID NO: 2) or by separate expression of OPRT activity (using, for example, the nucleotide sequence of SEQ ID NO: 4) and ODC activity (using, for example, the nucleotide sequence of SEQ ID NO: 6). In the absence of uridine, human UMPS^{-/-} cells will not survive without expression of OPRT and ODC activity, establishing the condition that both transgenes expressing both OPRT and ODC activity be present for maintenance and survival of the cells. In this example, placing OPRT and ODC independent functional domains under the control of separate expression control sequences enables use of more than one lineage-specific genes to select for the desired differentiated cell population. For example, OPRT can be delivered to a first locus and ODC can be delivered to a second locus. The first locus can be the auxotrophy-inducing locus. The second locus can be, for example, a safe harbor locus such as CCR5. In some embodiments, the second locus is targeted using CCR5 homology arms, wherein the homology arms are defined as a left and a right homology arm. An exemplary CCR5 left homology arm is defined as SEQ ID NO: 11. An exemplary CCR5 right homology arm is defined as SEQ ID NO: 12. Alternatively, both OPRT and ODC can be delivered to a safe harbor locus such as CCR5, for example, using CCR5 left and right homology arms of SEQ ID NO: 11 and SEQ ID NO: 12, respectively. OPRT can be under the expression control of a first expression control sequence regulated by, e.g., a first transcription factor specifically expressed in the desired differentiated cell population. Likewise, ODC can be under the expression control of a second expression control sequence regulated by, e.g., a second transcription factor specifically expressed in the desired differentiated cell population. In some embodiments, the OPRT and ODC sequences are independently linked to their respective first and second expression control sequences.

In some embodiments, the auxotrophic gene is human CAD (carbamoyl-phosphate synthetase 2, aspartate transcarbamylase, and dihydroorotase) (ENSG00000084774). Human CAD encodes a protein with three independent functional domains representing the first three enzymatic activities in the pyrimidine biosynthesis pathway. In some embodiments wherein the auxotrophic gene is human CAD, the first independent functional domain comprises carbamoyl-phosphate synthetase 2, the second independent functional domain comprises aspartate transcarbamylase, and the third independent functional domain comprises dihydroorotase. In the absence of uridine, human cells having inhibited CAD activity will not survive (see Swyryd, Elizabeth A., Sally S. Seaver, and George R. Stark. “N-(phosphonacetyl)-L-aspartate, a potent transition state analog inhibitor of aspartate transcarbamylase, blocks proliferation of mammalian cells in culture.” Journal of Biological Chemistry 249.21 (1974): 6945-6950, the contents of which are incorporated by reference in their entirety), establishing the condition that transgenes expressing each of carbamoyl-phosphate synthetase 2, aspartate transcarbamylase, and dihydroorotase be present for maintenance and survival of the cells. In this example, placing carbamoyl-phosphate synthetase 2, aspartate transcarbamylase, and dihydroorotase independent functional domains under the control of separate expression control sequences enables use of more three lineage-specific genes to select for the desired differentiated cell population. For example, carbamoyl-phosphate synthetase 2 can be delivered to a first locus, aspartate transcarbamylase can be delivered to a second locus, and dihydroorotase can be delivered to a third locus. The first locus can be the auxotrophy-inducing locus. The second and/or third locus can be, for example, a safe harbor locus such as CCR5. In some embodiments, the second locus is targeted using CCR5 homology arms, wherein the homology arms are defined as a left and a right homology arm. An exemplary CCR5 left homology arm is defined as SEQ ID NO: 12. An exemplary CCR5 right homology arm is defined as SEQ ID NO: 11. Alternatively, carbamoyl-phosphate synthetase 2, aspartate transcarbamylase, and dihydroorotase can each individually be delivered to a safe harbor locus such as CCR5, for example, using CCR5 left and right homology arms of SEQ ID NO: 11 and SEQ ID NO: 12, respectively. Carbamoyl-phosphate synthetase 2 can be under the expression control of a first expression control sequence regulated by, e.g., a first transcription factor specifically expressed in the desired differentiated cell population. Likewise, aspartate transcarbamylase can be under the expression control of a second expression control sequence regulated by, e.g., a second transcription factor specifically expressed in the desired differentiated cell population. Dihydroorotase can be under the expression control of a third expression control sequence regulated by, e.g., a third transcription factor specifically expressed in the desired differentiated cell population. In some embodiments, the carbamoyl-phosphate synthetase 2, aspartate transcarbamylase, and dihydroorotase sequences are independently linked to their respective first, second, and third expression control sequences.

Further contemplated herein is the knockout or down-regulation of more than one multi-domain auxotrophic gene. For example, knocking out or knocking down the function of both UMPS and CAD genes in a population of cells would enable selection for 5 different genetic modifications, e.g. transgene insertions, in the population of cells. In the absence of uridine as an auxotrophic factor, cells lacking both UMPS and CAD genes will require enzyme activity of each independent functional domain OPRT, ODC, carbamoyl-phosphate synthetase 2, aspartate transcarbamylase, and dihydroorotase, allowing for selection of 5 different genetic manipulations or transgene insertions independently capable of re-expressing each of the 5 independent functional domains.

Auxotrophic genes comprising more than one independent functional domains as described herein can be incorporated into the design of cellular Boolean switches. As used herein, a Boolean switch refers to a circuit that is designed to perform a logical operation based on one or more inputs and which produces an output. Logical operations performed by Boolean switches include but are not limited to, AND, OR, NOR, NAND, NOT, IMPLY, NIMPLY, XOR, and XNOR. For example, OR represents a scenario in which any of one or more inputs is required to produce an output; AND represents a scenario in which all of the inputs are required to generate an output; and NOT gates are inverters whose function is to invert the input. Compound Boolean switches that consist of multiple logical operations can also be generated. An example of a simple AND gate Boolean switch can comprise a human UMPS−/− cell having integrated a first transgene expressing OPRT and a second transgene expressing ODC, such that the presence of both OPRT activity AND ODC activity in the cell results in the cellular output of survival in the absence of uridine. In some embodiments, the provision of an auxotrophic factor OR the re-expression of a first independent functional domain AND a second independent functional domain comprises a compound Boolean switch requiring the satisfaction of one or more logical conditions to produce a cellular output. Logical conditions satisfactory to one or more Boolean switches (AND, OR, NOR, NAND, NOT, IMPLY, NIMPLY, XOR, and XNOR) can comprise, for example, presence/absence of an auxotrophic factor, presence/absence of one or more independent functional domains, presence/absence of one or more tissue-specific factors, and/or concentration/relative level/duration of the presence/absence of one or more auxotrophic factor, independent functional domains, or tissue-specific factors.

In some embodiments, the methods described herein include methods of generating a population of differentiated cells comprising contacting progenitor cells with a CRISPR/Cas system comprising a guide RNA (gRNA) targeting biallelically a portion of an auxotrophy-inducing gene. The targeting biallelically can knockout or knockdown the auxotrophy-inducing gene, for example by interrupting the open reading frame or a regulatory sequence, or by introducing a target sequence for protein or nucleotide suppression or degradation. In embodiments where the auxotrophy-inducing gene comprises at least a first and a second independent functional domain, knockout or knockdown of the gene results in the progenitor cells being auxotrophic for each independent functional domain. Upon inducing auxotrophy in the progenitor cells, a first homologous recombination construct and a second homologous recombination construct can be introduced into the cells, the first homologous recombination construct comprising a first tissue-specific promoter and at least a portion of the first independent functional domain of the auxotrophy-inducing gene, and the second homologous recombination construct comprising a second tissue-specific promoter and at least a portion of the second independent functional domain of the auxotrophy-inducing gene. The progenitor cells can be grown in the presence of the auxotrophic factor and differentiation of the cells can be stimulated to produce differentiated cells (e.g., a cell type or tissue) expressing the first and the second tissue-specific promoters, resulting in the first and the second homologous recombination constructs being expressed in the differentiated cells. In this way, removing the auxotrophic factor eliminates cells lacking the first and the second independent functional domains and selects for cells having both domains functionally integrated.

In some embodiments, the auxotrophy-inducing gene has 2 or more independent functional domains, e.g., 3, 4, or 5 independent functional domains, or more than 5 independent functional domains, and re-expressing each independent functional domain in the auxotrophic cells is required to alleviate the auxotrophy, thereby enabling for selection of cells that express 2, 3, 4, 5, or more tissue-specific promoters by modifying the cells with 2, 3, 4, 5, or more homologous recombination constructs expressing the different independent functional domains under the regulation of different tissue-specific promoters expressed in the desired differentiated cell type or tissue.

In some embodiments, the auxotrophy-inducing gene is uridine monophosphate synthase (UMPS), the first independent functional domain comprises orotate phosphoribosyltransferase (OPRT), and the second independent functional domain comprises orotidine 5′-phosphate decarboxylase (ODC).

In some embodiments, the auxotrophy-inducing gene is carbamoyl-phosphate synthetase 2, aspartate transcarbamylase, and dihydroorotase (CAD), the first independent functional domain comprises carbamoyl-phosphate synthetase 2, the second independent functional domain comprises aspartate transcarbamylase, and the third independent functional domain comprises dihydroorotase.

The methods can further comprise contacting the cells with 5-FOA.

One or more of the homologous recombination constructs can be insert into a safe harbor locus, e.g., CCR5. In some embodiments, the CCR5 locus can be targeted using homology arms, wherein the homology arms are defined as a left and a right homology arm. An exemplary CCR5 left homology arm is defined as SEQ ID NO: 11. An exemplary CCR5 right homology arm is defined as SEQ ID NO: 12.

The auxotrophic factor can be uridine.

In some embodiments, one or more of the homologous recombination constructs further comprise a nucleotide sequence encoding a therapeutic factor. One or more of the homologous recombination constructs can be polycistronic, e.g., with an internal ribosome entry site (IRES) or a peptide 2A sequence (P2A) separating, e.g., the coding sequence encoding the independent functional domain and the coding sequence encoding a therapeutic factor.

Example progenitor cells for use in the methods described herein include, but are not limited to, hematopoietic stem cells (HSCs), embryonic stem cells, transdifferentiated stem cells, neural progenitor cells, mesenchymal stem cells, osteoblasts, and cardiomyocytes.

Examples of differentiated cell types or tissues for use in the methods described herein include, but are not limited to adipose tissue, adrenal gland, ascites, bladder, blood, bone, bone marrow, brain, cervix, connective tissue, ear, embryonic tissue, esophagus, eye, heart, hematopoietic tissue, intestine, kidney, larynx, liver, lung, lymph, lymph node, mammary gland, mouth, muscle, nerve, ovary, pancreas, parathyroid, pharynx, pituitary gland, placenta, prostate, salivary gland, skin, spleen, stomach, testis, thymus, thyroid, tonsil, trachea, umbilical cord, uterus, endocrine, neuronal, and vascular.

In some embodiments, the differentiated cell is an immune cell, e.g., a T cell, a B cell, or a natural killer (NK) cell.

Examples of tissue-specific promoters for use in the methods described herein include, but are not limited to: WAS proximal promoter; CD4 mini-promoter/enhancer; CD2 locus control region; CD4 minimal promoter and proximal enhancer and silencer; CD4 mini-promoter/enhancer; GATA-1 enhancer HS2 within the LTR; Ankyrin-1 and α-spectrin promoters combined or not with HS-40, GATA-1, ARE and intron 8 enhancers; Ankyrin-1 promoter/β-globin HS-40 enhancer; GATA-1 enhancer HS1 to HS2 within the retroviral LTR; Hybrid cytomegalovirus (CMV) enhancer/β-actin promoter; MCH II-specific HLA-DR promoter; Fascin promoter (pFascin); Dectin-2 gene promoter; 5′ untranslated region from the DC-STAMP; Heavy chain intronic enhancer (Ep) and matrix attachment regions; CD19 promoter; Hybrid immunoglobulin promoter (Igk promoter, intronic Enhancer and 3′ enhancer from Ig genes); CD68L promoter and first intron; Glycoprotein Ibα promoter; Apolipoprotein E (Apo E) enhancer/alpha1-antitrypsin (hAAT) promoter (ApoE/hAAT); HAAT promoter/Apo E locus control region; Albumin promoter; HAAT promoter/four copies of the Apo E enhancer; Albumin and hAAT promoters/al-microglobulin and prothrombin enhancers; HAAT promoter/Apo E locus control region; hAAT promoter/four copies of the Apo E enhancer; TBG promoter (thyroid hormone-binding globulin promoter and α1-microglobulin/bikunin enhancer); DC172 promoter (α1-antitrypsin promoter and α1-microglobulin enhancer); LCAT, kLSP-IVS, ApoE/hAAT and liver-fatty acid-binding protein promoters; RU486-responsive promoter; Creatine kinase promoter; Creatine kinase promoter; Synthetic muscle-specific promoter C5-12; Creatine kinase promoter; Hybrid enhancer/promoter regions of α-myosin and creatine kinase (MHCK7); Hybrid enhancer/promoter regions of α-myosin and creatine kinase; Synthetic muscle-specific promoter C5-12; Cardiac troponin-1 proximal promoter; E-selectin and KDR promoters; Prepro-endothelin-1 promoter; KDR promoter/hypoxia-responsive element; Flt-1 promoter; Flt-1 promoter; ICAM-2 promoter; Synthetic endothelial promoter; Endothelin-1 gene promoter; Amylase promoter; Insulin and human pdx-1 promoters; TRE-regulated insulin promoter; Enolase promoter; Enolase promoter; TRE-regulated synapsin promoter; Synapsin 1 promoter; PDGF-β promoter/CMV enhancer; PDGF-β, synapsin, tubulin-α and Ca2+/calmodulin-PK2 promoters combined with CMV enhancer; Phosphate-activated glutaminase and vesicular glutamate transporter-1 promoters; Glutamic acid decarboxylase-67 promoter; Tyrosine hydroxylase promoter; Neurofilament heavy gene promoter; Human red opsin promoter; Keratin-18 promoter; keratin-14 (K14) promoter; and Keratin-5 promoter.

In some embodiments, one or more of the homologous recombination constructs further comprises a nucleotide sequence encoding a conditional destabilization domain or a conditional ribozyme switch. In this manner, the auxotrophy of the modified cells described herein can be further regulated by triggering a condition for destabilization of an independent functional domain or a condition for degradation of a message RNA encoding an independent functional domain. The condition can be, for example, the presence of a ligand that stabilizes the destabilization domain, or the absence of the ligand thereby inducing destabilization and degradation of the independent functional domain.

The differentiated population of cells generated using the methods described herein can be administered to a subject. In some embodiments, the differentiated cells are immune cells carrying a therapeutic factor and the subject is in need of or suspected to be in need of the therapeutic factor.

Also provided are methods of alleviating auxotrophy comprising providing a plurality of auxotrophic progenitor cells which have been generated by knockout or knockdown of an auxotrophy-inducing gene, wherein the gene comprises at least a first and a second independent functional domain, and inserting into the genome of the auxotrophic progenitor cells a first construct comprising an open reading frame of the first independent functional domain into a first tissue-specific gene locus, and inserting a second construct comprising an open reading frame of the second independent functional domain into a second tissue-specific gene locus. In some embodiments, expression of the tissue-specific genes at the first and second loci is not disrupted. Thus, auxotrophy is thereby alleviated upon differentiation of the progenitor cells into a cell type or tissue expressing the first and the second tissue-specific genes at the first and second loci.

Exploitation of auxotrophy-inducing genes comprising more than 2 independent functional domains is contemplated. For example, the auxotrophy-inducing gene can comprise, 2, 3, 4, 5, or more independent functional domains, such the re-expression of each of the 2, 3, 4, 5, or more independent functional domains is required to alleviate auxotrophy. Where the respective independent functional domains are inserted into the genome of the auxotrophic progenitor cells at respective tissue-specific gene loci, only cells expressing tissue-specific promoters corresponding to each of the first, second, third, fourth, and/or fifth tissue-specific loci having integrated respective independent functional domains will survive removal of the auxotrophic factor.

In some embodiments, the progenitor cells are induced pluripotent stem cells (iPSCs) or embryonic stem cells (ESCs).

The auxotrophy-inducing gene can be uridine monophosphate synthase (UMPS), the first independent functional domain comprises orotate phosphoribosyltransferase (OPRT), and the second independent functional domain comprises orotidine 5′-phosphate decarboxylase (ODC).

The auxotrophy-inducing gene can be carbamoyl-phosphate synthetase 2, aspartate transcarbamylase, and dihydroorotase (CAD), the first independent functional domain comprises carbamoyl-phosphate synthetase 2, the second independent functional domain comprises aspartate transcarbamylase, and the third independent functional domain comprises dihydroorotase.

One or more of the constructs can be polycistronic additionally encoding, for example, a therapeutic factor and further comprising an internal ribosome entry site (IRES) or a peptide 2A sequence (P2A) regulating expression of the cistrons of the construct(s).

In some embodiments, the tissue-specific gene locus is an insulin locus.

In some embodiments, the differentiated cell is an immune cell, e.g., a T cell, a B cell, or a natural killer (NK) cell.

In some embodiments, the tissue-specific gene is not replaced during the inserting step.

In some embodiments, differentiated cells produce insulin.

One or more of the constructs can comprise a nucleotide sequence encoding a conditional destabilization domain or a conditional ribozyme switch.

Also provided are methods of selecting cells having functionally integrated at least 2 exogenous genes. The methods can comprise providing a plurality of cells with a knockout or knockdown of an auxotrophy-inducing gene comprising at least a first and a second independent functional domain, resulting in auxotrophy for an auxotrophic factor in the plurality of cells. The cells can be grown in a medium providing the auxotrophic factor, and can be transfected with a first delivery system comprising a nucleotide sequence encoding the first exogenous gene and a nucleotide sequence encoding the first independent functional domain and a second delivery system comprising a nucleotide sequence encoding the second exogenous gene and a nucleotide sequence encoding the second independent functional domain. Upon replacement of the medium with a medium lacking the auxotrophic factor, cells that have not functionally integrated both the first and the second exogenous genes will remain auxotrophic and will not persist in culture, thereby selecting for cells that have functionally integrated the first and second delivery systems.

The methods described further contemplate exploitation of auxotrophy-inducing genes have additional independent functional domains, e.g., auxotrophy-inducing genes having 2, 3, 4, 5, or more independent functional domains, such that re-expression of each of the independent functional domains is required to alleviate auxotrophy in the modified cells.

The methods can comprise transfecting the plurality of cells with, a delivery system corresponding to each functional domain of the auxotrophy-inducing gene, wherein each delivery system comprises a nucleotide sequence encoding an exogenous gene and a nucleotide sequence encoding an independent functional domain. One or more of the delivery systems can be a plasmid, a lentivirus, an adeno-associated virus (AAV), or a nanoparticle.

In some embodiments, the auxotrophy-inducing gene is uridine monophosphate synthase (UMPS), the first independent functional domain comprises orotate phosphoribosyltransferase (OPRT), and the second independent functional domain comprises orotidine 5′-phosphate decarboxylase (ODC).

In some embodiments, the auxotrophy-inducing gene is carbamoyl-phosphate synthetase 2, aspartate transcarbamylase, and dihydroorotase (CAD), the first independent functional domain comprises carbamoyl-phosphate synthetase 2, the second independent functional domain comprises aspartate transcarbamylase, and the third independent functional domain comprises dihydroorotase.

### Methods of Selecting for Mature Beta Cells

Also provided are methods of generating a population of mature human beta cells comprising contacting a plurality of progenitor cells with a CRISPR/Cas system comprising a gRNA targeting biallelically a portion of a human UMPS gene resulting in the progenitor cells being auxotrophic for uridine. Alternatively, the methods can comprise knocking down or otherwise knocking out a human UMPS gene using non-CRISPR-based methodologies. The methods can further comprise contacting the plurality of progenitor cells with a first homologous recombination construct and a second homologous recombination construct, the first homologous recombination construct comprising a nucleotide sequence encoding insulin (ENSG00000254647, or a portion thereof) or an insulin-dependent expression control sequence operably linked to a first independent functional domain of UMPS, and the second homologous recombination construct comprising a nucleotide sequence encoding Nkx6.1 (ENSG00000163623, or a portion thereof) or an Nkx6.1-dependent expression control sequence operably linked to a second independent functional domain of UMPS. In some embodiments, the first and the second independent functional domains are selected from OPRT and ODC and are expressed only in cells expressing both insulin and Nkx6.1. Non-homologous recombination-based transgene insertion is also contemplated for use in the methods described herein. The cells can be grown in the presence of uridine until the and beyond the time the recombination constructs are introduced into the cells. In the presence of uridine, the cells can be stimulated into mature beta cells, using, for example the methods described in Ma, Haiting, et al. “Establishment of human pluripotent stem cell-derived pancreatic β-like cells in the mouse pancreas.” Proceedings of the National Academy of Sciences 115.15 (2018): 3924-3929; Pagliuca, Felicia W., et al. “Generation of functional human pancreatic β cells in vitro.” Cell 159.2 (2014): 428-439; and/or Rezania, Alireza, et al. “Reversal of diabetes with insulin-producing cells derived in vitro from human pluripotent stem cells.” Nature biotechnology 32.11 (2014): 1121; the disclosure of each of which is incorporated by reference herein in its entirety. The methods can further comprise selecting for mature beta cells expressing both insulin and Nkx6.1 by removing uridine. Uridine withdrawal or removal under these circumstances will inhibit proliferation or survival of cells that do not express both insulin and Nkx6.1.

In some embodiments, the one or more of the split auxotrophy constructs inserted with the independent functional domain transgene(s) can further comprise a therapeutic factor or a gene encoding a therapeutic factor. The therapeutic factor can be expressed as a cassette with targeted auxotrophy-inducing gene or portion thereof. Expression of the constructs, including the therapeutic factor, can be optimized by creating a polycistronic construct having, for example, a linker between two or more expressed components, wherein the linker is an internal ribosome entry site (IRES) or a peptide 2A sequence (P2A) or the like. Exemplary linker sequences are provided as SEQ ID NOs: 20, 22, 24, 25, 26, 27, 28, 29, and 30.

Expression of the re-expressed auxotrophy-inducing gene(s), independent functional domains thereof, or other transgene in some embodiments can be regulated by a eukaryotic promoter sequence such as EF1a (SEQ ID NO: 31 or SEQ ID NO: 32). Bicistronic or multicistronic constructs can be prepared by separating the expressed components of the construct with linkers as described or using an internal ribosome entry site (IRES) such as that of SEQ ID NO: 33.

Termination and polyadenylation signal sequences can be used to terminate and stabilize the transcripts produced from the constructs described herein. In some embodiments, transcription is terminated and stabilized using a bovine growth hormone (bGH) poly-adenylation signal sequence, such as that of SEQ ID NO: 39 or 40.

In some embodiments, the methods described herein are useful in alleviating type 1 diabetes in a subject. The methods can comprise administering to the subject the mature human beta cells produced by the methods described herein.

Mature human beta cells selected from a population of in vitro differentiated progenitor cells are also provided. The mature human beta cells can comprise a biallelic genetic modification of an auxotrophy-inducing gene resulting in auxotrophy for an auxotrophic factor as described herein. The mature human beta cells can further comprise one or more transgenes re-expressing the auxotrophy-inducing gene or one or more independent functional domains of the auxotrophy-inducing gene, such that the cells can survive after successful integration of the transgenes upon removal of the auxotrophic factor. In some embodiments, the mature human beta cells have a genetic manipulation of auxotrophy-inducing gene UMPS. Thus, the auxotrophic factor can be uridine, the independent functional domains can be selected from OPRT and ODC, and the one or more transgenes can further comprise a nucleotide sequence encoding insulin or an insulin-dependent expression control sequence and a nucleotide sequence encoding Nkx6.1 or an Nkx6.1-dependent expression control sequence. Where the OPRT and ODC independent functional domains are operably linked to expression of insulin and Nkx6.1, respectively as the case may be, cells expressing both insulin and Nkx6.1 will also express OPRT and ODC, and will effectively re-express the auxotrophy-inducing gene, thereby remaining viable even after withdrawal of uridine from the culture medium.

### Methods of Selecting for Differentiated Megakaryocytes

In some embodiments, the methods described herein are useful for generating engineered megakaryocytes and/or engineered platelets. The engineered megakaryocytes and/or engineered platelets can express a payload, e.g., a protein of interest, which can be a therapeutic protein or polypeptide. Megakaryocytes engineered and produced according to the selection methods described herein can express the payload, which is subsequently loaded into platelets produced from the megakaryocytes. Similarly, platelets engineered and produced according to the selection methods described herein can be loaded with a therapeutic payload for delivery to, e.g., a subject in need of the therapeutic effects of the payload.

In some embodiments, the engineered megakaryocytes and/or engineered platelets express a first payload and a second payload. The first and/or the second payload(s) can be a therapeutic, e.g., a therapeutic protein or polypeptide. Megakaryocytes engineered and produced according to the selection methods described herein can express the first and/or second payloads, which are subsequently loaded into platelets produced from the megakaryocytes. Similarly, platelets engineered and produced according to the selection methods described herein can be loaded with first and/or second therapeutic payload(s) for delivery to, e.g., a subject in need of the therapeutic effects of the payload(s).

In some embodiments, payloads as described herein may include, for example, factor VIII as a therapeutic for hemophelia as described in Du, Lily M., et al. “Platelet-targeted gene therapy with human factor VIII establishes haemostasis in dogs with haemophilia A.” Nature communications 4.1 (2013): 1-11, the contents of which are incorporated herein by reference in their entirety. In some embodiments, payloads as described herein may include, for example, PD-1 or anti-PD-L1 antibody to target engineered platelets to PD-L1-expressing tumor cells as described in Zhang, Xudong, et al. “Engineering PD-1-presenting platelets for cancer immunotherapy.” Nano letters 18.9 (2018): 5716-5725, the contents of which are incorporated herein by reference in their entirety.

Use of a megakaryocyte-specific promoter permits cell-type-specific expression of the payload(s) in megakaryocytes and/or platelets. Examples of megakaryocyte-specific promoters include, for example, human PGK, Pf4, GP1BA, GP6, or GP9 promoters (see, e.g., Latorre-Rey, L. J., et al. “Targeting expression to megakaryocytes and platelets by lineage-specific lentiviral vectors.” Journal of Thrombosis and Haemostasis 15.2 (2017): 341-355, incorporated herein by reference in its entirety) as well as CD68L promoter, glycoprotein Ibα promoter, and integrin αIIb promoter (see Tables 3 and 4).

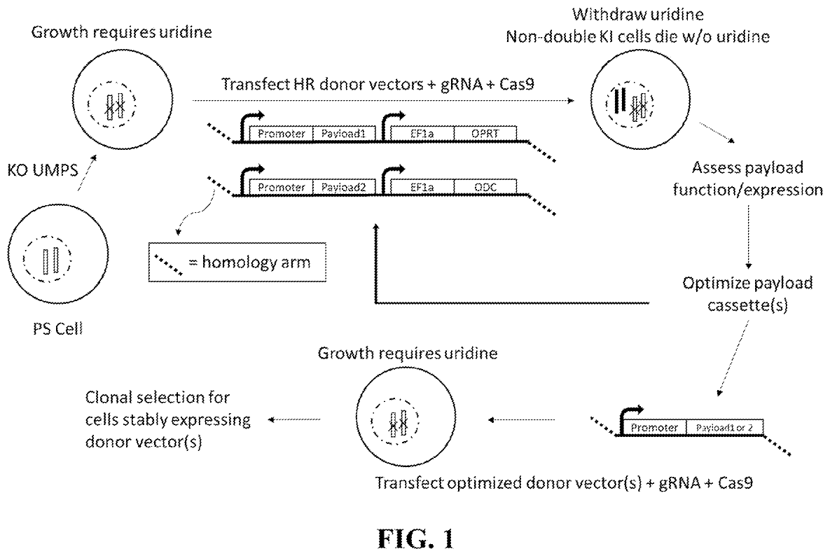

The selection methods provided herein enable purification and/or enrichment of populations of engineered megakaryocytes and platelets. For example, FIG. 1 shows a schematic of an example process using split auxotrophic selection for optimizing expression vectors for use in PS cell-derived engineered megakaryocytes. Progenitor cells such as pluripotent stem (“PS”) cells are engineered to be UMPS knockout (“KO UMPS”) using, e.g., CRISPR-based or other genetic engineering systems. UMPS knockout cells are cultured in uridine to promote survival and growth, and are transfected, e.g., electroporated, with homologous recombination (HR) donor vectors (also referred to herein as a first and a second expression construct), guide RNA (“gRNA”), and Cas9 for inserting donor vectors/expression constructs into, e.g., a safe harbor locus such as CCR5, yielding double knock-in (KI) cells which require uridine for survival and/or growth. HR donor vectors/expression constructs include first expression cassettes comprising a nucleotide sequence encoding a first payload driven by a megakaryocyte-specific promoter and a nucleotide sequence encoding a second payload driven by a megakaryocyte-specific promoter (depicted in FIG. 1 as “Payload1” and “Payload2,” respectively, each driven by “Promoter”). HR donor vectors/expression constructs contain second expression cassettes including a first independent functional domain of UMPS and a second independent functional domain of UMPS, respectively, such that UMPS is functionally re-expressed alongside Payload1 and Payload2 in cells bearing double KI under conditions sufficient to drive expression of a megakaryocyte-specific promoter. UMPS independent functional domains can be under transcriptional regulatory control of, for example, a constitutive mammalian promoter such as EF1a such that the UMPS independent functional domains are constitutively expressed. Functionality of polypeptides expressed from HR donor vectors/expression constructs (i.e., Payload1, Payload2, UMPS first independent functional domain, and/or UMPS second independent functional domain) can be assessed. Examples of assessing functionality of polypeptides expressed from HR donor vectors/expression constructs include detecting DNA corresponding to donor vectors/expression constructs (e.g., PCR), detecting RNA corresponding to donor vector transcription (e.g., rtPCR), detecting protein corresponding to donor vector expression (e.g., Western blot, immunocytology, cell sorting, etc.), or analyzing cellular morphology and/or function for evidence of functional expression of donor vectors. Optimized expression cassettes (e.g., for Payload1 alone, for Payload2 alone, or for Payload1 and Payload2) can then be generated and used for creation of cell lines stably expressing the payloads under control of the tissue-specific promoter, e.g., megakaryocyte-specific promoter.

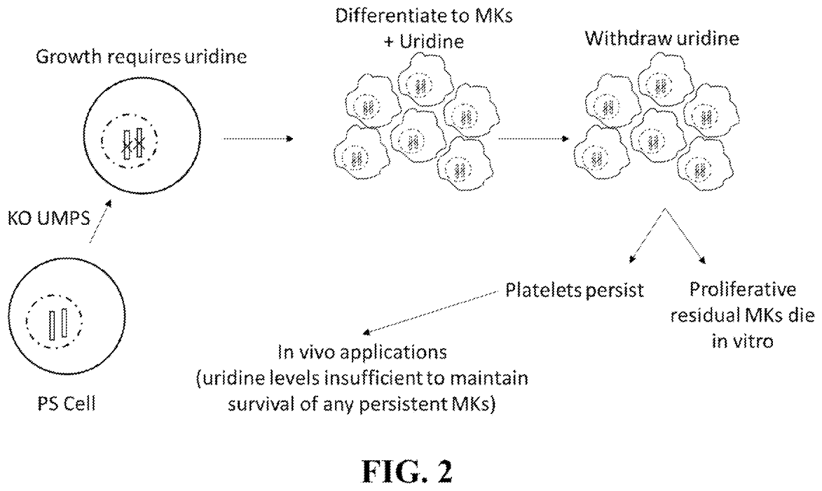

Additionally, FIG. 2 shows a schematic of an example process using uridine auxotrophy-based selection methods to generate platelets for in vivo applications from UMPS knockout (KO) pluripotent stem (PS) cells which have been differentiated in vitro to megakaryocytes (MKs). In one embodiment of the example process depicted in FIG. 2, nucleated and/or proliferative cells (including, for example, residual PS cells and/or proliferative megakaryocytes) die or fail to propagate after differentiation when uridine is withdrawn from the culture conditions. Platelets produced from the megakaryocytes persist in culture. The platelets can be used in downstream in vivo applications. In some embodiments, the methods produce a substantially pure population of platelets devoid or substantially devoid of proliferative cells. Upon administration to a subject, the platelets produced by the megakaryocytes remain functional, while any residual PS or megakaryocytes or other nucleated or proliferative cells die in vivo or fail to propagate due their being auxotrophic for uridine. In some embodiments, endogenous uridine levels in vivo are insufficient to maintain viability of any residual PS or megakaryocytes or other nucleated or proliferative cells following administration to a subject. In some embodiments, the auxotrophic nature of the cells permits only non-nucleated, non-proliferative platelets to persist.

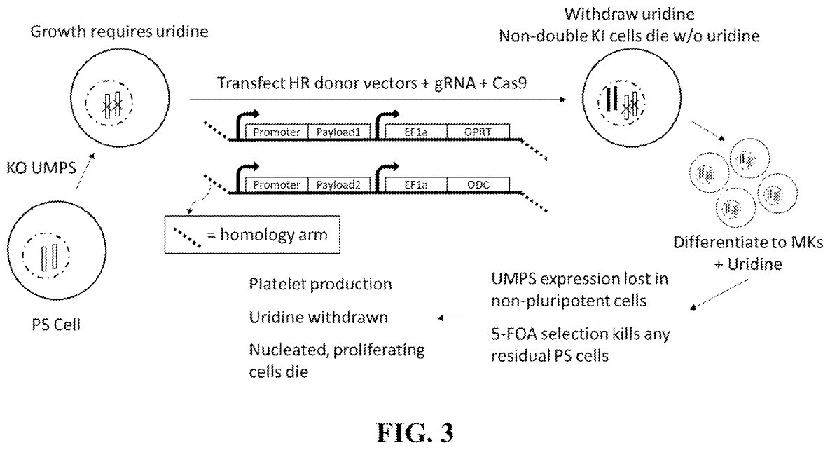

FIG. 3 shows a schematic of another embodiment using split auxotrophy to produce engineered platelets in vitro from pluripotent stem (PS) cells. Pluripotent stem (“PS”) cells are engineered to be UMPS knockouts (“KO UMPS”) using, e.g., CRISPR-based or other genetic engineering systems. UMPS knockout cells are cultured in uridine to promote survival and growth, and are transfected, e.g., electroporated, with homologous recombination (HR) donor vectors (e.g., expression constructs), guide RNA (“gRNA”), and Cas9 for inserting donor vectors into, e.g., a safe harbor locus such as CCR5, yielding double knock-in (KI) cells which require uridine for survival and/or growth. The first HR donor vector/expression construct includes a first expression cassette comprising a nucleotide sequence encoding a first payload (“Payload1”) driven by a megakaryocyte-specific promoter and a second expression cassette comprising a nucleotide sequence encoding a first independent functional domain of UMPS. The second HR donor vector/expression construct includes a third expression cassette encoding a nucleotide sequence encoding a second payload (“Payload2”) driven by a megakaryocyte-specific promoter (“Promoter”) and a fourth expression cassette including a nucleotide sequence encoding a second independent functional domain of UMPS, such that UMPS is functionally re-expressed alongside first and second payloads in cells bearing double KI under conditions sufficient to drive expression of a megakaryocyte-specific promoter. In some embodiments, UMPS independent functional domains can be under transcriptional regulatory control of, for example, a constitutive mammalian promoter such as EF1a such that the UMPS independent functional domains are constitutively expressed. Double knock-in cells are differentiated in vitro to megakaryocytes (MKs) in the presence of uridine to ensure survival of double knock-in cells. UMPS expression, e.g., expression of OPRT and ODC independent functional domains, is lost in differentiated cells. 5-FOA selection can be used to eliminate residual pluripotent cells. In some embodiments, platelets produced by the megakaryocytes are loaded with expressed payload polypeptides. In some embodiments, platelets produced by the megakaryocytes persist after uridine withdrawal, whereas nucleated or proliferating cells such as any residual PS cells or megakaryocytes die or fail to propagate after withdrawal of uridine.

Megakaryocytes produced and selected for according to the methods described herein can be engineered megakaryocytes. Engineered megakaryocytes can include, for example, nucleotide sequences encoding a payload. The payload can be, for example, a nucleotide sequence encoding a therapeutic RNA such as an antisense RNA, siRNAs, aptamers, microRNA mimics/anti-miRs and synthetic mRNA. The payload can be, for example, a nucleotide sequence encoding a payload polypeptide sequence. The payload polypeptide sequence can be, for example, a polypeptide to be delivered in vivo. The polypeptide can be a therapeutic polypeptide.

The methods provided herein can be used to generate substantially pure populations of functionally mature platelets. The substantially pure populations of platelets can be devoid or substantially devoid of nucleated and/or proliferative cells. The substantially pure populations of platelets according to the present disclosure can be administered to a subject. Upon administration to a subject, any residual proliferative and/or nucleated cells, such as residual non-differentiated cells, residual progenitor/pluripotent stem cells, or residual megakaryocytes which remain nucleated or proliferative will die in vivo due to the lack of a functional UMPS or other auxotrophy-inducing gene. In some embodiments, in vivo endogenous levels of uridine or other auxotrophic factor is insufficient to sustain cells engineered to be auxotrophic for uridine or other auxotrophic factor. Hence, administration of a population of cells produced according to the methods of the present description are non-viable, cannot proliferate, and/or cannot survive upon administration to a subject.

### C. Therapeutic Methods

Use of the cells described in the present disclosure for treatment of a disease, disorder, or condition is also encompassed.

Certain embodiments provide the disease, the disorder, or the condition as selected from the group consisting of cancer, Parkinson's disease, graft versus host disease (GvHD), autoimmune conditions, hyperproliferative disorder or condition, malignant transformation, liver conditions, genetic conditions including inherited genetic defects, juvenile onset diabetes mellitus and ocular compartment conditions.

In certain embodiments, the disease, the disorder, or the condition affects at least one system of the body selected from the group consisting of muscular, skeletal, circulatory, nervous, lymphatic, respiratory endocrine, digestive, excretory, and reproductive systems. Conditions that affect more than one cell type in the subject may be treated with more than one embodiment of the cells described in the present disclosure with each cell line activated by a different auxotrophic factor.

Certain embodiments provide the cell line as regenerative. In an aspect of the present disclosure, the subject may be contacted with more than one cell and/or with one or more auxotrophic factor. Certain embodiments provide localized release of the auxotrophic factor, e.g. nutrient or the enzyme. Alternative embodiments provide systemic delivery. For example, localized release is affected via utilization of a biocompatible device. In an aspect of the present disclosure, the biocompatible device may restrict diffusion of the cell line in the subject. Certain embodiments of the method provide removing the auxotrophic factor to deplete therapeutic effects of the modified host cell in the subject or to induce cell death in the modified host cell. Certain embodiments of the method provide the therapeutic effects as including at least one selected from the group consisting of: molecule trafficking, inducing cell death, cell death, and recruiting of additional cells. Certain embodiments of the method provide that the unmodified host cells are derived from the same subject prior to treatment of the subject with the modified host cells.

The disclosure contemplates kits comprising such compositions or components of such compositions, optionally with a container or vial.

The methods described herein can be used to select for cells that have differentiated into a particular tissue. The tissue can be one selected from the group consisting of: adipose tissue, adrenal gland, ascites, bladder, blood, bone, bone marrow, brain, cervix, connective tissue, ear, embryonic tissue, esophagus, eye, heart, hematopoietic tissue, intestine, kidney, larynx, liver, lung, lymph, lymph node, mammary gland, mouth, muscle, nerve, ovary, pancreas, parathyroid, pharynx, pituitary gland, placenta, prostate, salivary gland, skin, spleen, stomach, testis, thymus, thyroid, tonsil, trachea, umbilical cord, uterus, endocrine, neuronal tissue, and vascular tissue. In some embodiments, the differentiated cell is an immune cell, and the immune cell can be differentiated into, for example, a T cell, a B cell, or a natural killer (NK) cell.

The differentiated population of cells generated using the methods described herein can be administered to a subject. In some embodiments, the differentiated cells are immune cells carrying a therapeutic factor and the subject is in need of or suspected to be in need of the therapeutic factor.

As an example, the methods described herein are useful in alleviating type 1 diabetes in a subject. The methods can comprise administering to the subject the mature human beta cells produced by the methods described herein.

### D. Methods for Drug Screening

In addition to therapeutic methods, the differentiated cell populations produced using the methods described herein can be useful for drug screening in vitro. Known methods for preparing differentiated cell populations are hampered by inadequate methods of differentiating progenitor cells into desired differentiated cell or tissue types and/or inadequate methods of selecting for differentiated cells from a population of progenitor cells. (See, for example, Goversen, Birgit, et al. “The immature electrophysiological phenotype of iPSC-CMs still hampers in vitro drug screening: Special focus on IK1.” Pharmacology & therapeutics 183 (2018): 127-136, the disclosure of which is incorporated by reference herein in its entirety.) The methods of selecting for differentiated cell populations from a population of in vitro differentiated progenitor cells, therefore, can be used to improve efficiency and efficacy of in vitro drug screening methodologies. Candidate drugs or drug libraries can be applied to populations of differentiated cells to determine efficacy, tolerability, toxicity, dosage, bioavailability, absorption, half-life, molecular interactions, adverse effects, metabolic effects, genetic effects, physiological effects, electrophysiological effects, or other outcomes of drug exposure to the cell type of interest. In one embodiment, for example, candidate drug(s) or drug libraries can be administered to iPSC-derived cardiomyocytes or cardiomyocyte sub-populations differentiated and selected for using the methods described herein to determine drug outcomes in the specified cellular subtype. For instance, the differentiation methods described can be used to select for first heart field lineage cells which can be further differentiated into ventricular cardiomyocytes for in vitro testing of drugs in this sub-population. In other embodiments, the differentiation methods described herein can be used to select for epicardial lineage cells which can be further differentiated into nodal cardiomyocytes for in vitro drug testing in this sub-population. In other embodiments, the differentiation methods described herein can be used to select for second heart field lineage cells which can be further differentiated into atrial cardiomyocytes for in vitro drug testing in this sub-population. In still further embodiments, the differentiation methods described herein can be used to select for endothelial cells which can be for in vitro drug testing in this sub-population.

### IV. Pharmaceutical Compositions

Disclosed herein, in some embodiments, are methods, compositions and kits for use of the modified cells, including pharmaceutical compositions, therapeutic methods, and methods of administration of auxotrophic factors to control—increase, decrease or cease—the growth and reproduction of the modified cells and to control the expression of the therapeutic factor by the transgene. Further, the methods, compositions, and kits described herein may also be used for selection of transfected cells and generating a differentiated population of cells.

The modified mammalian host cell may be administered to the subject separately from the auxotrophic factor or in combination with the auxotrophic factor. Although the descriptions of pharmaceutical compositions provided herein are principally directed to pharmaceutical compositions which are suitable for administration to humans, it will be understood by the skilled artisan that such compositions are generally suitable for administration to any animals.

Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys. In some embodiments, compositions are administered to humans, human patients, or subjects.

In some instances, the pharmaceutical compositions described herein is used in a method of treating a disease, a disorder, or a condition in a subject, the method including: (i) generating a cell line which is auxotrophic for a nutrient, an enzyme, an altered pH, an altered temperature, an altered concentration of a moiety, and/or a niche environment, such that the nutrient, enzyme, altered pH, altered temperature, and niche environment is not present in the subject; (ii) contacting the subject with the resulting auxotrophic cell line of step (i); (iii) contacting the subject of (ii) with the auxotrophic factor which is selected from the nutrient, enzyme, moiety that alters pH and/or temperature, and a cellular niche environment in the subject, such that the auxotrophic factor activates the auxotrophic system or element resulting in the growth of the cell line and/or the expression of one or more therapeutic entities for the subject.

The pharmaceutical compositions of the present disclosure may also be used in a method of treating a disease, a disorder, or a condition in a subject, comprising (a) administering to the subject a modified host cell according to the present disclosure, and (b) administering the auxotrophic factor to the subject in an amount sufficient to promote growth of the modified host cell.

Compositions comprising a nutrient auxotrophic factor may also be used for administration to a human comprising a modified cell of the present disclosure.

Many present pharmaceutical compositions comprising stem cells are likely to give the patient cancer; therefore, a cell population needs to be differentiated. The methods described herein provide a purely differentiated cell population that does not contain any stem cells for administration to a patient.

### V. Formulations

The modified host cell is genetically engineered to insert the construct with a transgene encoding the therapeutic factor into the auxotrophy-inducing locus. Delivery of Cas9 protein/gRNA ribonucleoprotein complexes (Cas9 RNPs) targeting the desired locus may be performed by liposome-mediated transfection, electroporation, or nuclear localization. In some embodiments, the modified host cell is in contact with a medium containing serum following electroporation. In some embodiments, the modified host cell is in contact with a medium containing reduced serum or containing no serum following electroporation.

The modified host cell or auxotrophic factor of the present disclosure may be formulated using one or more excipients to: (1) increase stability; (2) alter the biodistribution (e.g., target the cell line to specific tissues or cell types); (3) alter the release profile of an encoded therapeutic factor; and/or (4) improve uptake of the auxotrophic factor.

Formulations of the present disclosure can include, without limitation, saline, liposomes, lipid nanoparticles, polymers, peptides, proteins, and combinations thereof.

Formulations of the pharmaceutical compositions described herein may be prepared by any method known or hereafter developed in the art of pharmacology. As used herein the term “pharmaceutical composition” refers to compositions including at least one active ingredient and optionally one or more pharmaceutically acceptable excipients. Pharmaceutical compositions of the present disclosure may be sterile.

In general, such preparatory methods include the step of associating the active ingredient with an excipient and/or one or more other accessory ingredients. As used herein, the phrase “active ingredient” generally refers to either (a) a modified host cell or construct including a transgene capable of expressing a therapeutic factor inserted into an auxotrophy-inducing locus, or (b) the corresponding auxotrophic factor, or (c) the nuclease system for targeting cleavage within the auxotrophy-inducing locus.

Formulations of the modified host cell or the auxotrophic factor and pharmaceutical compositions described herein may be prepared by a variety of methods known in the art. In some embodiments, a population of differentiated cells generated using the methods described herein may be administered to a subject.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition including a predetermined amount of the active ingredient.

Relative amounts of the active ingredient (e.g. the modified host cell or auxotrophic factor), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%, e.g., between 0.5 and 50%, between 1-30%, between 5-80%, or at least 80% (w/w) active ingredient.

A. Excipients and Diluents

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

B. Inactive Ingredients

In some embodiments, formulations may include at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present disclosure may be approved by the U.S. Food and Drug Administration (FDA).

C. Pharmaceutically Acceptable Salts

The auxotrophic factor may be administered as a pharmaceutically acceptable salt thereof. As used herein, “pharmaceutically acceptable salts” refers to derivatives of the disclosed compounds such that the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecyl sulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids.

### VI. Dosing and Administration

The modified host cells or auxotrophic factors of the present disclosure included in the pharmaceutical compositions described above may be administered by any delivery route, systemic delivery or local delivery, which results in a therapeutically effective outcome. These include, but are not limited to, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal, (through the eye), intracavernous injection (into a pathologic cavity), intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), or in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, and spinal.

A. Parenteral and Injectable Administration

In some embodiments, the cells described herein may be administered parenterally.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

B. Depot Administration

As described herein, in some embodiments, pharmaceutical compositions including the modified host cell of the present disclosure are formulated in depots for extended release. Generally, specific organs or tissues (“target tissues”) are targeted for administration. In some embodiments, localized release is affected via utilization of a biocompatible device. For example, the biocompatible device may restrict diffusion of the cell line in the subject.

In some aspects of the present disclosure, pharmaceutical compositions including the modified host cell of the present disclosure are spatially retained within or proximal to target tissues. Provided are methods of providing pharmaceutical compositions including the modified host cell or the auxotrophic factor, to target tissues of mammalian subjects by contacting target tissues (which include one or more target cells) with pharmaceutical compositions including the modified host cell or the auxotrophic factor, under conditions such that they are substantially retained in target tissues, meaning that at least 10, 20, 30, 40, 50, 60, 70, 80, 85, 90, 95, 96, 97, 98, 99, 99.9, 99.99, or greater than 99.99% of the composition is retained in the target tissues. For example, at least 1%, 5%, 10%, 20%, 30%, 40%, 50%, 60%, 70%, 80%, 85%, 90%, 95%, 96%, 97%, 98%, 99%, 99.9%, 99.99% or greater than 99.99% of pharmaceutical compositions including the modified host cell or the auxotrophic factor administered to subjects are present at a period of time following administration.

Certain aspects of the present disclosure are directed to methods of providing pharmaceutical compositions including the modified host cell or the auxotrophic factor of the present disclosure to target tissues of mammalian subjects, by contacting target tissues with pharmaceutical compositions including the modified host cell under conditions such that they are substantially retained in such target tissues. Pharmaceutical compositions including the modified host cell include enough active ingredient such that the effect of interest is produced in at least one target cell. In some embodiments, pharmaceutical compositions including the modified host cell generally include one or more cell penetration agents, although “naked” formulations (such as without cell penetration agents or other agents) are also contemplated, with or without pharmaceutically acceptable excipients.

C. Dose and Regimen

The present disclosure provides methods of administering modified host cells or auxotrophic factors in accordance with the present disclosure to a subject in need thereof. The pharmaceutical compositions including the cells described herein or the auxotrophic factor and compositions of the present disclosure may be administered to a subject using any amount and any route of administration effective for preventing, treating, managing, or diagnosing diseases, disorders and/or conditions. The exact amount required will vary from subject to subject, depending on the species, age, and general condition of the subject, the severity of the disease, the particular composition, its mode of administration, its mode of activity, and the like. The subject may be a human, a mammal, or an animal. The specific therapeutically effective, prophylactically effective, or appropriate diagnostic dose level for any particular individual will depend upon a variety of factors including the disorder being treated and the severity of the disorder; the activity of the specific payload employed; the specific composition employed; the age, body weight, general health, sex and diet of the patient; the time of administration, route of administration, and rate of excretion of the auxotrophic factor; the duration of the treatment; drugs used in combination or coincidental with the specific modified host cell or auxotrophic factor employed; and like factors well known in the medical arts.

In certain embodiments, cells described herein or the auxotrophic factor pharmaceutical compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect.

In certain embodiments, the cell described herein or auxotrophic factor pharmaceutical compositions in accordance with the present disclosure may be administered at about 10 to about 600 μl/site, 50 to about 500 μl/site, 100 to about 400 μl/site, 120 to about 300 μl/site, 140 to about 200 μl/site, about 160 μl/site. As non-limiting examples, the modified host cell or auxotrophic factor may be administered at 50 μl/site and/or 150 μl/site.

The desired dosage of the modified host cell or auxotrophic factor of the present disclosure may be delivered only once, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

The desired dosage of the cells of the present disclosure may be administered one time or multiple times. The auxotrophic factor is administered regularly with a set frequency over a period of time, or continuously as a “continuous flow”. A total daily dose, an amount given or prescribed in 24-hour period, may be administered by any of these methods, or as a combination of these methods.

In some embodiments, delivery of the cell(s) or auxotrophic factor of the present disclosure to a subject provides a therapeutic effect for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

The cells of the present disclosure may be used in combination with one or more other therapeutic, prophylactic, research or diagnostic agents, or medical procedures, either sequentially or concurrently. In general, each agent will be administered at a dose and/or on a time schedule determined for that agent. In some embodiments, the present disclosure encompasses the delivery of pharmaceutical, prophylactic, research, or diagnostic compositions in combination with agents that may improve their bioavailability, reduce and/or modify their metabolism, inhibit their excretion, and/or modify their distribution within the body.

For example, the cells of the present disclosure or auxotrophic factor is administered as a biocompatible device that restricts diffusion in the subject to increase bioavailability in the area targeted for treatment. The cell(s) of the present disclosure or auxotrophic factor may also be administered by local delivery.

The term “conditioning regime” refers to a course of therapy that a patient undergoes before stem cell transplantation. For example, before hematopoietic stem cell transplantation, a patient may undergo myeloablative therapy, non-myeloablative therapy or reduced intensity conditioning to prevent rejection of the stem cell transplant even if the stem cell originated from the same patient. The conditioning regime may involve administration of cytotoxic agents. The conditioning regime may also include immunosuppression, antibodies, and irradiation. Other possible conditioning regiments include antibody mediated conditioning (see e.g., Czechowicz et al., 318(5854) Science 1296-9 (2007); Palchaudari et al., 34(7) Nature Biotechnology 738-745 (2016); Chhabra et al., 10:8 (351) Science Translational Medicine 351ra105 (2016)) and CAR-T mediated conditioning (see, e.g., Arai et al., 26(5) Molecular Therapy 1181-1197 (2018); each of which is hereby incorporated by reference in its entirety). Conditioning needs to be used create space in the brain for microglia derived from engineered HSCs to migrate into to deliver the protein of interest (recent gene therapy trials for ALD and MLD). The conditioning regimen is also designed to create niche “space” to allow the transplanted cells to have a place in the body to engraft and proliferate. In hematopoietic stem cell transplantation, for example, the conditioning regimen creates niche space in the bone marrow for the transplanted hematopoietic stem cells to engraft into. Without a conditioning regimen the transplanted hematopoietic stem cells cannot engraft. In some embodiments, the cell lines are T cells that are genetically engineered to be auxotrophic. Engineered auxotrophic T cells may be used as CAR T cells to act as a living drug and administered to a patient along with an auxotrophic factor to condition the patient for a hematopoietic stem cell transplant. Prior to the delivery of the donor hematopoietic stem cells, the auxotrophic factor may be removed, which results in the elimination of the engineered auxotrophic T cells. In some embodiments, the cell lines are allogeneic T cells that are genetically engineered to be auxotrophic. Engineered auxotrophic allogeneic T cells may be administered to a patient along with an auxotrophic factor to provide a therapeutic effect. Upon the patient developing graft-versus-host disease (GvHD), the auxotrophic factor may be removed, which results in the elimination of the engineered auxotrophic allogeneic T cells which have become alloreactive.

Use of the cells described in the present disclosure for treatment of a disease, disorder, or condition is also encompassed by the disclosure.

Certain embodiments provide the disease, the disorder, or the condition as selected from the group consisting of cancer, Parkinson's disease, graft versus host disease (GvHD), autoimmune conditions, hyperproliferative disorder or condition, malignant transformation, liver conditions, genetic conditions including inherited genetic defects, juvenile onset diabetes mellitus and ocular compartment conditions.

In certain embodiments, the disease, the disorder, or the condition affects at least one system of the body selected from the group consisting of muscular, skeletal, circulatory, nervous, lymphatic, respiratory endocrine, digestive, excretory, and reproductive systems. Conditions that affect more than one cell type in the subject may be treated with more than one embodiment of the cells described in the present disclosure with each cell line activated by a different auxotrophic factor.

Certain embodiments provide the cell line as regenerative. In an aspect of the present disclosure, the subject may be contacted with more than one cell and/or with one or more auxotrophic factor. Certain embodiments provide localized release of the auxotrophic factor, e.g. nutrient or the enzyme. Alternative embodiments provide systemic delivery. For example, localized release is affected via utilization of a biocompatible device. In an aspect of the present disclosure, the biocompatible device may restrict diffusion of the cell line in the subject. Certain embodiments of the method provide removing the auxotrophic factor to deplete therapeutic effects of the modified host cell in the subject or to induce cell death in the modified host cell. Certain embodiments of the method provide the therapeutic effects as including at least one selected from the group consisting of: molecule trafficking, inducing cell death, cell death, and recruiting of additional cells. Certain embodiments of the method provide that the unmodified host cells are derived from the same subject prior to treatment of the subject with the modified host cells.

The disclosure contemplates kits comprising such compositions or components of such compositions, optionally with a container or vial.

### VII. Definitions

The term “about” in relation to a numerical value x means, for example, x+10%.

The term “active ingredient” generally refers to the ingredient in a composition that is involved in exerting a therapeutic effect. As used herein, it generally refers to (a) the modified host cell or construct including a transgene as described herein, (b) the corresponding auxotrophic factor as described herein, or (c) the nuclease system for targeting cleavage within the auxotrophy-inducing locus.

The term “altered concentration” as used herein, refers to an increase in concentration of an auxotrophic factor compared to the concentration of the auxotrophic factor in the subject prior to administration of the pharmaceutical compositions described herein.

The term “altered pH” as used herein, refers to a change in pH induced in a subject compared to the pH in the subject prior to administration of the pharmaceutical composition described herein.

The term “altered temperature” as used herein refers to a change in temperature induced in a subject compared to the temperature in the subject prior to administration of the pharmaceutical composition as described herein.

The term “auxotrophy” or “auxotrophic” as used herein, refers to a condition of a cell that requires the exogenous administration of an auxotrophic factor to sustain growth and reproduction of the cell.

The term “auxotrophy-inducing locus” or “auxotrophy-inducing gene” as used herein refers to a region of a chromosome in a cell that, when disrupted, causes the cell to be auxotrophic. For example, a cell can be rendered auxotrophic by disrupting a gene encoding an enzyme involved in synthesis, recycling or salvage of an auxotrophic factor (either directly or upstream through synthesizing intermediates used to make the auxotrophic factor), or by disrupting an expression control sequence that regulates the gene's expression without disrupting the open reading frame of the auxotrophy-inducing gene.

The term “bioavailability” as used herein, refers to systemic availability of a given amount of the modified host cell or auxotrophic factor administered to a subject.

The term “Cas9” as used herein, refers to CRISPR-associated protein 9, which is an endonuclease for use in genome editing.

The term “comprising” means “including” as well as “consisting” e.g. a composition “comprising” X may consist exclusively of X or may include something additional e.g. X+Y.

The term “conditioning regime” refers to a course of therapy that a patient undergoes before stem cell transplantation.

The term “continuous flow” as used herein, refers to a dose of therapeutic administered continuously for a period of time in a single route/single point of contact, i.e., continuous administration event.

The term “CRISPR” as used herein, refers to clustered regularly interspaced short palindromic repeats of DNA that deploy an enzyme that cuts the RNA nucleotides of an invading cell.

The term “CRISPR/Cas9 nuclease system” as used herein, refers to a genetic engineering tool that includes a guide RNA (gRNA) sequence with a binding site for Cas9 and a targeting sequence specific for the site to be cleaved in the target DNA. The Cas9 binds the gRNA to form a ribonucleoprotein complex that binds and cleaves the target site.

The term “expanding” when used in the context of cells refers to increasing the number of cells through generation of progeny.

The term “expression control sequence” refers to a nucleotide sequence capable of regulating or controlling expression of a nucleotide sequence of interest. Examples include a promoter, enhancer, transcription factor binding site, miRNA binding site, and the like.

The term “homologous recombination” (HR) refers to insertion of a nucleotide sequence during repair of breaks in DNA via homology-directed repair mechanisms. This process uses a “donor” molecule or “donor template” with homology to nucleotide sequence in the region of the break as a template for repairing the break. The inserted nucleotide sequence can be a single base change in the genome or the insertion of large sequence of DNA. The donor template can comprise one or more expression constructs comprising one or more nucleotide sequence encoding one or more functional components of an expression construct, e.g., encoding an mRNA or a polypeptide payload. For example, a “homologous recombination donor vector” (and like terms) as used herein refers to a donor molecule or donor template nucleic acid molecule which is incorporated or designed to be incorporated into a genome of a cell via homologous recombination. An expression construct can be polycistronic. In some embodiments, an expression construct and or an expression cassette within an expression construct comprises one or more linker sequences, e.g., an internal ribosome entry site (IRES) or a peptide 2A sequence (P2A), T2A (collectively, a “2A sequence”) or the like.

The term “expression construct” refers to a nucleotide sequence comprising the sequence elements necessary for expression in a eukaryotic cell, e.g., promoter sequence and a coding sequence. In some cases, an expression construct includes one or more “expression cassettes,” each expression cassette comprising an independent promoter operably linked to an independent coding sequence. The coding sequences referred to herein can code for DNA, RNA, or polypeptide payloads, for example.

The term “payload” as used herein refers to a biomolecule, e.g., a DNA, an RNA, or a polypeptide biomolecule. For example, a payload can be a therapeutic biomolecule. A payload can be, for example, an antisense RNA, an siRNA, an aptamer, a microRNA mimic, an anti-miR, a synthetic mRNA, or a polypeptide. In some embodiments, a payload acts within a cell to achieve a desired cellular function. In some embodiments, a payload acts at the surface of cell to achieve a desired cellular function. In some embodiments, a payload acts externally of a cell to achieve a desired cellular function. In some embodiments, a payload acts cell-intrinsically to achieve a desired cellular function. In some embodiments, a payload acts cell-extrinsically to achieve a desired cellular function.

The term “progenitor cell” as used herein refers to, for example, stem cells, embryonic stem cells (ESCs), pluripotent stem (PS) cells (PSCs), induced pluripotent stem (iPS) cells (iPSCs), hematopoietic stem cells (HSCs), somatic stem cells, transdifferentiated stem cells, differentiated cells, mesenchymal stem cells or mesenchymal stromal cells, neural progenitor cells or neural stem cells, hematopoietic stem cells or hematopoietic progenitor cells, adipose stem cells, keratinocytes, osteoblasts, skeletal stem cells, muscle stem cells, cardiomyocytes, fibroblasts, NK cells, B-cells, T cells, peripheral blood mononuclear cells (PBMCs).

The term “homologous” or “homology,” when used in the context of two or more nucleotide sequences, refers to a degree of base pairing or hybridization that is sufficient to specifically bind the two nucleotide sequences together in a cell under physiologic conditions. Homology can also be described by calculating the percentage of nucleotides that would undergo Watson-Crick base pairing with the complementary sequence, e.g. at least 70% identity, preferably at least 75%, 80%, 85%, 90%, 91%, 92%, 93%, 94%, 95%, 96%, 97%, 98%, 99%, or higher identity over a specified number of bases. With respect to donor templates, for example, the homology may be over 200-400 bases. With respect to guide sequences, for example, the homology may be over 15-20 bases.

The term “independent functional domain” refers to individual domains of a protein which each contribute a function to the full protein. For instance, certain proteins in nature comprise an enzyme or catalytic domain which is structurally and functionally distinct from the remainder of the protein. Such enzyme or catalytic domain can be deemed an independent functional domain if, when expressed separately and independently from the remainder of the protein sequence, it retains its enzymatic or catalytic activity under normal cellular or physiological conditions. “Independent functional domain” can also refer to individual subunits of a protein which each contribute a function to the full protein. Independent functional domains can be individual domains, subunits, or fragments of proteins which are expressed from a single gene, yet have an independent function that is separable from or is a component of the overall function of the gene/protein from which it derives.

The term “operatively linked” refers to functional linkage between a nucleic acid expression control sequence (such as a promoter, enhancer, signal sequence, or array of transcription factor binding sites) and a second nucleic acid sequence, wherein the expression control sequence affects transcription and/or translation of the second nucleic acid sequence.

The term “pharmaceutical composition” as used herein, refers to a composition including at least one active ingredient and optionally one or more pharmaceutically acceptable excipients.

The term “pharmaceutically acceptable salt” as used herein, refers to derivatives of the disclosed compounds such that the parent compound is modified by converting an existing acid or base moiety to its salt form (e.g., by reacting the free base group with a suitable organic acid). All references herein to compounds or components include the pharmaceutically acceptable salt thereof.

The term “regenerative” as used herein, refers to renewal or restoration of an organ or system of the subject.

The term “re-expression” as used herein, for example in the context of “re-expression of an auxotrophy-inducing gene,” refers to the expression of a transgene that replaces, rescues, supplements, or augments the expression of gene, e.g., an auxotrophy-inducing gene, in a cell.

The term “tissue-specific factor” as used herein refers to a gene or protein or a combination of genes or proteins that is/are uniquely or differentially expressed in differentiated cells of a particular tissue. In some instances, tissue-specific factors are genes or proteins that are uniquely or differentially expressed in a cell type that is an intermediate of a particular desired cell fate. The presence of a certain tissue-specific factor or a certain combination of tissue-specific factors in a cell or tissue can thus identify the cell or tissue as differentiated according to a desired cell fate or tissue type.

The term “tissue-specific promoter” as used herein refers to a nucleotide regulatory sequence that drives expression of a gene in a specific tissue or cell type. Various exemplary tissue-specific promoters are provided in Table 3. In some instances, the presence of a tissue-specific factor in a specific tissue or cell type drives expression of a target gene regulated by the corresponding tissue-specific promoter.

The term “therapeutic factor” refers to a product encoded by the inserted transgene that treats and/or alleviates symptoms of the disease, disorder, or condition of the subject.

The term “therapeutic amount” refers to an amount of therapeutic factor sufficient to exert a “therapeutic effect”, which means an alleviation or amelioration of symptoms of the disease, disorder or condition.

The term “unit dose” as used herein, refers to a discrete amount of the pharmaceutical composition including a predetermined amount of the active ingredient.

The details of one or more embodiments of the present disclosure are set forth in the accompanying description below. Although any materials and methods similar or equivalent to those described herein can be used in the practice or testing of the present disclosure, the preferred materials and methods are now described. Other features, objects and advantages of the present disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this present disclosure belongs. In the case of conflict, the present description will control.

The present disclosure is further illustrated by the following non-limiting examples.

### EXAMPLES

### Example 1. Culturing of Stem Cells

UMPS/uridine auxotrophy is engineered in human pluripotent cells. The modified host cells that are the subject matter of the disclosure herein may include stem cells that are maintained and differentiated using the techniques below as shown in U.S. Pat. No. 8,945,862, which is hereby incorporated by reference in its entirety.

Undifferentiated hESCs (H9 line from WICELL®, passages 35 to 45) are grown on an inactivated mouse embryonic fibroblast (MEF) feeder layer (Stem Cells, 2007. 25(2): p. 392-401, which is hereby incorporated by reference in its entirety). The cell is maintained at an undifferentiated stage on irradiated low-passage MEF feeder layers on 0.1% gelatin-coated plates. The medium is changed daily. The medium consists of Dulbecco's Modified Eagle Medium (DMEM)/F-12, 20% knockout serum replacement, 0.1 mM non-essential amino acids, 2 mM L-glutamine, 0.1 mM β-mercaptoethanol, and 4 ng/ml rhFGF-2 (R&D Systems Inc., Minneapolis). The undifferentiated hESCs are treated by 1 mg/ml collagenase type IV in DMEM/F12 and scraped mechanically on the day of passage. Prior to differentiation, hESCs are seeded onto MATRIGEL® protein mixture (Corning, Inc.)-coated plates in conditioned medium (CM) prepared from MEF as follows (Nat Biotechnol, 2001. 19(10): p. 971-4, which is hereby incorporated by reference in its entirety). MEF cells were harvested and irradiated with 50 Gy, and were cultured with hES medium without basic fibroblast growth factor (bFGF). CM is collected daily and supplemented with an additional 4 ng/ml of bFGF before feeding hES cells.

### Example 2. Insertion of Construct in to Auxotrophic Cells

The UMPS locus is disrupted in the hESCs by electroporation of Cas9 RNP to insert an expression control sequence comprising a tissue-specific promoter into the genomic locus. The promoter will begin to instigate transcription due to interaction with endothelial tissue. For gene editing, hESCs are treated with 10 μm ROCK inhibitor (Y-27632) for 24 hours before electroporation. Cells at 70-80% confluence are harvested with ACCUTASE® solution (Life Technologies). 500,000 cells were used per reaction with a SpCas9 concentration of 150 μg/mL (Integrated DNA Technologies) and a Cas9:sgRNA molar ratio of 1:3 and electroporation performed in P3 Primary Cell solution (Lonza) in 16-well NUCLEOCUVETTE™ Strips in the 4D NUCLEOFECTOR system (Lonza). Immediately after electroporation, cells are transferred into one well of a MATRIGEL® protein mixture (Corning, Inc.)-coated 24 well plate containing 500 μl of mTeSR™ media (STEMCELL Technologies) with 10 μM Y-27632. Media was changed 24 hours after editing and Y-27632 is removed 48 hours after.

### Example 3. In Vitro Differentiation of Human Embryonic Stem Cell (ESC)-Endothelial Cells (ECs)

To induce hESC differentiation, undifferentiated hESCs are cultured in differentiation medium containing Iscove's Modified Dulbecco's Medium (IMDM) and 15% defined fetal bovine serum (FBS) (Hyclone, Logan, Utah), 0.1 mM non-essential amino acids, 2 mM L-glutamine, 450 μM monothioglycerol (Sigma, St. Louis, Mo.), 50 U/ml penicillin, and 50 μg/ml streptomycin, either in ultra-low attachment plates for the formation of suspended embryoid bodies (EBs) as previously described (see, Proc Natl Acad Sci USA, 2002. 99(7): p. 4391-6 and Stem Cells, 2007. 25(2): p. 392-401; each of which is hereby incorporated by reference in its entirety). Briefly, hESCs cultured on MATRIGEL® protein mixture (Corning, Inc.) coated plate with conditioned media were treated by 2 mg/ml dispase (Invitrogen, Carlsbad, Calif.) for 15 minutes at 37° C. to loosen the colonies. The colonies were then scraped off and transferred into ultra-low-attachment plates (Corning Incorporated, Corning, N.Y.) for embryoid body formation.

### Example 4. Selection of a Pure Population of Differentiated Cells

Cells are grown in 5-FOA and uridine sources. 5-FOA is removed prior to start of differentiation. Once differentiation is performed as described in Example 3, the uridine is removed from the medium. Cells not expressing UMPS under the control of the inserted tissue-specific promoter die, thereby leaving a pure population of differentiated cells.

### Example 5. Selecting for Cells Re-Expressing UMPS in a UMPS−/− Background

Insertion of a construct expressing UMPS into a cell population having UMPS gene biallelically knocked out as described herein demonstrates selection in principle using the methods described herein. A construct (SEQ ID NO: 42) expressing mCherry and UMPS separated by a 2A linker sequence under control of a constitutive promoter (EF1a) delivered via DNA vector was inserted by electroporation into the CCR5 locus targeted using homologous recombination arms. Cells were grown in the presence of uridine to alleviate any selection pressure, then uridine was removed to select for cells successfully re-expressing UMPS. Cells were sorted by expression of mCherry using flow cytometry. After 14 days in culture, % mCherry+ cells was approximately 20% in the presence of uridine; % mCherry+ cells was approximately 90% in the absence of uridine. Thus, re-expression of UMPS in UMPS knockout cells demonstrates auxotrophy-based cellular selection.

### Example 6. Split Auxotrophy

Independent functional domains of UMPS, i.e. OPRT and ODC, are reintroduced into UMPS knockout cells according to Example 2 using two separate vectors. A first homologous recombination donor vector carrying OPRT and mCherry (SEQ ID NO: 43) comprises sequence encoding OPRT (SEQ ID NO: 4, encoding amino acid sequence of SEQ ID NO: 5), 2A linker (SEQ ID NO: 19, SEQ ID NO: 21, or SEQ ID NO: 23, encoding amino acid sequences of SEQ ID NO: 20, SEQ ID NO: 22, and SEQ ID NO: 24, respectively), and sequence encoding mCherry fluorescent protein (SEQ ID NO: 34 or SEQ ID NO: 35, each encoding amino acid sequence of SEQ ID NO: 36). Homologous recombination donor vector carrying ODC and CD19 (SEQ ID NO: 44) comprises sequence encoding tCD19 (SEQ ID NO: 37, encoding amino acid sequence of SEQ ID NO: 38), 2A linker, and sequence encoding ODC (SEQ ID NO: 6, encoding amino acid sequence of SEQ ID NO: 7). Expression of OPRT and ODC constructs can be driven by a eukaryotic promoter such as EF1a (SEQ ID NO: 31 or SEQ ID NO: 32). Homologous recombination vectors are co-targeted for integration into safe harbor locus CCR5 (using, for example, CCR5 left and right homology arms selected from SEQ ID NOs: 11-15 as described herein). Homologous recombination vectors can further include termination signals such as a bGH-PolyA termination signal (SEQ ID NO: 39 or SEQ ID NO: 40). Cells are cultured with and without uridine, and the percent of cells expressing both CD19 and mCherry (CD19+/mCherry+) is measured by flow cytometry over time. Results at day 16 are provided in Table 5.

TABLE 5
Split UMPS allows for selection of dual transgene integration

| | No Uridine | +Uridine |
|---|---|---|
| % CD19⁻/mCherry⁻ | 0.1 | 92.9 |
| % CD19⁺ only | 0.1 | 1.2 |
| % mCherry⁺ only | 5.1 | 5.6 |
| % CD19⁺/mCherry⁺ | 94.7 | 0.3 |

As shown in Table 5, withdrawal of uridine enriches CD19+/mCherry+ cells in culture of UMPS knockout cells, indicating that absence of uridine applies selection pressure for cells expressing dual transgenes that together replace UMPS function.

### Example 7. Split Auxotrophic System for Selection of Mature Beta Cells

Mature beta cells are marked by expression of one or more tissue- or cell-type specific factors. For instance, co-expression of insulin (“INS,” ENSG00000254647) and NKX6.1 (ENSG00000163623) indicates mature stem-cell-derived beta cells. (See, e.g., Ma, Haiting, et al. “Establishment of human pluripotent stem cell-derived pancreatic β-like cells in the mouse pancreas.” Proceedings of the National Academy of Sciences 115.15 (2018): 3924-3929; Pagliuca, Felicia W., et al. “Generation of functional human pancreatic β cells in vitro.” Cell 159.2 (2014): 428-439; and Rezania, Alireza, et al. “Reversal of diabetes with insulin-producing cells derived in vitro from human pluripotent stem cells.” Nature biotechnology 32.11 (2014): 1121; the disclosure of each of which is incorporated by reference herein in its entirety.) According to Rezania et al., “PDX1 (a pancreatic homeodomain transcription factor [ENSG00000139515]) and NKX6.1 (a homeobox transcription factor) are co-expressed in multipotent pancreatic progenitor cells, which give rise to all adult pancreatic endoderm cells,” and their co-expression is restricted to beta cells. NEUROD1 (ENSG00000162992), NKX2.2 (ENSG00000125820), and MAFA (ENSG00000182759) represent additional genes whose expression is limited to or differentially expressed in mature beta cells. (See, for example, Nishimura, Wataru, Satoru Takahashi, and Kazuki Yasuda. “MafA is critical for maintenance of the mature beta cell phenotype in mice.” Diabetologia 58.3 (2015): 566-574, the disclosure of which is incorporated by reference herein in its entirety.) Rezania et al. and Pagliuca et al. further detail multi-stage differentiation procedures to prepare mature beta cells from human pluripotent stem cells with expression profiles for each stage of differentiation, and show that thus-differentiated cells reverse diabetes in vivo.

It is now specifically contemplated that mature beta cells can be specifically selected for from a population of in vitro differentiated human cells by, for example, starting with UMPS/uridine auxotrophic human iPSCs and inserting transgenes expressing, for example, insulin or an insulin-dependent transgene, wherein the transgene is regulated by endogenous insulin expression control sequences and further comprises UMPS. The insulin gene can be targeted for homologous recombination using a homologous recombination vector carrying mCherry and UMPS (SEQ ID NO: 41), which vector comprises left homology arm comprising SEQ ID NO: 16 and/or SEQ ID NO: 17 and a right homology arm comprising SEQ ID NO: 18. Left and right homology arms of SEQ ID NO: 16 and SEQ ID NO: 18, respectively, rely on a nuclease system targeting a position in the insulin coding sequence that is upstream of the portion of the insulin coding sequence of SEQ ID NO: 17. The transgene inserted into the insulin locus can further comprise, for example, an IRES-driven mCherry reporter, wherein the IRES comprises SEQ ID NO: 33 or like sequence and mCherry comprises SEQ ID NO: 34 or SEQ ID NO: 35. Expression of the reporter protein can be linked to UMPS expression by providing a nucleotide sequence encoding UMPS (SEQ ID NO: 1 or SEQ ID NO: 2, encoding amino acid sequence of SEQ ID NO: 3). The UMPS coding sequence can be separated from the reporter using, for example, a T2A linker as described elsewhere herein. Thus, the INS-mCherry-UMPS construct comprises a tricistronic construct expressing insulin, mCherry, and UMPS under endogenous insulin regulatory control sequences. Expression of the tricistronic cassette can be terminated by including termination signals such as a bGH-PolyA termination signal (SEQ ID NO: 39 or SEQ ID NO: 40) following UMPS coding sequences.

Insertion of the INS-mCherry-UMPS transgene under control of endogenous insulin expression control sequences into the uridine-auxotrophic cells enables re-expression of UMPS only in cells that express insulin, i.e., mature beta cells. Thus, the methods described herein include methods of selecting for mature beta cells from a population of cells using single auxotrophic selection, where “single” refers to the use of one auxotrophy-inducing gene (i.e., UMPS).

The methods described herein further include dual-specific selection methods using split auxotrophy. For example, mature beta cells can be specifically selected for from a population of in vitro differentiated human cells by, for example, starting with UMPS/uridine auxotrophic human iPSCs and inserting a first transgene expressing, for example, insulin or an insulin-dependent transgene, wherein the transgene is regulated by endogenous insulin expression control sequences and further comprises a first UMPS independent functional domain (e.g., ODC). Cells expressing only ODC, for example, will remain auxotrophic for uridine. Therefore, a second transgene expressing, for example, OPRT, must be expressed to relieve auxotrophy and permit withdrawal of uridine. The expression of the second transgene can be under regulation of endogenous expression control sequences native to a second mature beta cell-specific factor, e.g., NKX6.1. Thus, upon withdrawal of the auxotrophic factor uridine from the population of in vitro differentiated cells, only those cells expressing both insulin and NKX6.1 will survive, thereby selecting for mature beta cells.

Similarly, it is specifically contemplated that mature beta cells can be specifically selected for from a population of in vitro differentiated human cells by, for example, starting with UMPS/uridine auxotrophic human iPSCs and inserting transgenes expressing, for example, insulin or an insulin-dependent transgene comprising a first UMPS independent functional domain (e.g., ODC) and MAFA or a MAFA-dependent transgene comprising a second UMPS independent functional domain (e.g., OPRT). Upon withdrawal of the auxotrophic factor uridine from the population of in vitro differentiated cells, only those cells expressing both insulin and MAFA will survive, thereby selecting for mature beta cells.

It is contemplated that cells selected for and enriched using the single and dual-specific selection methods described herein can be administered in vivo to alleviate diabetes in subjects in need of glucose-sensitive mature insulin-producing beta cells, including, for example, subjects having type 1 diabetes.

The methods of selecting for mature beta cells as provided herein are summarized in Table 6 below. As shown in Table 6, starting with UMPS/uridine auxotrophic human iPSCs, one or more beta cell-specific factors and/or one or more beta cell-specific promoters can be coopted to re-express the auxotrophic gene such that, upon uridine withdrawal, only cells expressing the beta cell-specific genes, and thus only mature beta cells, will survive and/or propagate.

TABLE 6
Methods for Selection of Differentiated Cells Using Auxotrophic
Selection Methods and Split Auxotrophic Selection Methods

| | Single auxotrophic selection | Dual specific auxotrophic selection |
|---|---|---|
| Step 1 | Generate iPSCs with Genetic Background: UMPS^{-/-} | |
| Step 2 | Propagate iPSCs in the presence of uridine | |
| Step 3 | Insert UMPS re-expression cassette | Insert first UMPS independent functional |
| | under control of a beta cell-specific | domain cassette under control of a first |
| | promoter | beta cell-specific promoter |
| | - or - | - or - |
| | Insert UMPS re-expression cassette | Insert first UMPS independent functional |
| | further comprising a beta cell-specific | domain cassette further comprising a first |
| | factor at the locus of the beta cell- | beta cell-specific factor at the locus of |
| | specific factor | the beta cell-specific factor |
| Step | | Insert second UMPS independent |
| 3b | | functional domain cassette under control |
| | | of a second beta cell-specific promoter |
| | | - or - |
| | | Insert second UMPS independent |
| | | functional domain cassette further |
| | | comprising a second beta cell-specific |
| | | factor at the locus of the second beta cell- |
| | | specific factor |

| | |
|---|---|
| Step 4 | Differentiate iPSCs to mature beta cell fate |
| Step 5 | Remove uridine to select for differentiated cells |

In the single auxotrophic selection context, re-expression of UMPS can be regulated by cellular expression of insulin, NEUROD1, NKX2.2, and/or MAFA to select for cells differentiated into mature beta cells.

In the dual-specific auxotrophic selection context, re-expression of ODC and OPRT can be regulated by cellular expression of insulin and NKX6.1, respectively; insulin and NEUROD1, respectively; insulin and NKX2.2, respectively; insulin and MAFA, respectively; or insulin and PDX1, respectively, to select for cells differentiated into mature beta cells. Alternatively, re-expression of OPRT and ODC can be regulated by cellular expression of insulin and NKX6.1, respectively; insulin and NEUROD1, respectively; insulin and NKX2.2, respectively; insulin and MAFA, respectively; or insulin and PDX1, respectively, to select for cells differentiated into mature beta cells.

As a proof of concept, UMPS knockout cells were engineered to express insulin and were differentiated to pancreatic progenitor cells using an appropriate method as described, for example, in Ma et al. (2018), Pagliuca et al. (2014), and/or Rezania et al. (2014) discussed and incorporated herein. The UMPS gene was knocked out in H9 human embryonic stem cells (hESCs) according to the methods described herein. A GFP-Luciferase expression construct under regulation of a constitutive promoter was integrated by homologous recombination into the HBB locus (see, for example, Dever, Daniel P., et al. “CRISPR/Cas9 β-globin gene targeting in human haematopoietic stem cells.” Nature 539.7629 (2016): 384-389, the contents of which are incorporated herein by reference in their entirety). A second homologous recombination donor vector carrying an mCherry-UMPS expression cassette operably linked to a coding sequence of the N-terminal portion of insulin (the INS-mCherry-UMPS construct described above) was integrated into the insulin locus in-frame, such that insulin, mCherry, and UMPS are all expressed from the modified insulin locus. GFP expression was verified in the cells to be constitutively expressed. The cells were subjected to a differentiation protocol whereby at day 1 following start of culture, Stage 1 of hESC differentiation was initiated to produce definitive endodermal cells. On day 3, Stage 2 was initiated to differentiate definitive endodermal cells to primitive gut tube cells. On day 6, Stage 3 was initiated to differentiate primitive gut tube cells to posterior foregut cells. On day 9, Stage 4 was initiated to differentiate posterior foregut cells to pancreatic progenitor cells around day 14. Cells were monitored and assessed for mCherry and UMPS expression on subsequent days up to day 25. The differentiation protocol was followed with cells either in the continuous presence of uridine through day 18 or through the end of the culture period, or through only day 12 (during Stage 4). mCherry and UMPS expression were assessed. An mCherry on vs. off threshold was defined to identify UMPS expressing cells (given UMPS expression is coupled to mCherry) using thresholding based on fluorescence images. Because GFP expression was from a ubiquitous promoter and was found to be independent of the cell's differentiation state. Thresholded mCherry-off cells can be considered undifferentiated, whilst mCherry-on cells can be considered to be differentiating into beta cells (as mCherry and UMPS expression are driven by the insulin promoter, a beta cell-specific gene).

In this experiment, uridine withdrawal (at day 12) should inhibit cell growth (and gene expression) in cells that do not express UMPS (and therefore do not express mCherry). Therefore, expression of GFP in mCherry negative cells, when uridine is present, was expected to be higher than the expression of GFP in mCherry negative cells when uridine is absent. Therefore, the ratio of [mean GFP(mCherry-Off)/mean GFP(mCherry-On)] in uridine withdrawn-conditions was compared to the ratio of [mean GFP(mCherry-Off)/mean GFP(mCherry-On)] in uridine-continued conditions. The results are shown in Table 7, where the “Auxotrophy Metric” was calculated as the mean GFP ratio in uridine-withdrawn conditions divided by the mean GFP ratio in uridine-continued conditions: [(mean GFP(mCherry-Off)/mean GFP(mCherry-On)) uridine-withdrawn-conditions]/[(mean GFP(mCherry-Off)/mean GFP(mCherry-On)) uridine-continued conditions]. This ratio was calculated across multiple fields of view from microscopy images, and the results demonstrated that uridine withdrawal inhibits GFP expression in UMPS/mCherry non-expressing, and therefore non-differentiated, cells. This demonstrates that lineage specific auxotrophy can be used to select for differentiated cells.

TABLE 7
Decreased GFP protein expression in non-insulin (UMPS)
expressing cells in uridine-withdrawn conditions

| Culture Day: | 12 | 18 | 25 |
|---|---|---|---|
| Auxotrophy Metric | 0.9904 | 0.9827 | 0.7261 |

It is contemplated that cells selected for and enriched in this manner can be administered in vivo to alleviate diabetes in subjects in need of glucose-sensitive mature insulin-producing beta cells, including, for example, subjects having type 1 diabetes. The use of auxotrophic selection methods in conjunction with the differentiation of mature beta cells can improve the purity, quantity, and efficacy of in vitro-differentiated mature beta cells, for example, for administration to subjects in need.

### Example 8. Differentiation of Defined Subsets of Cardiomyocytes

Using the paradigm set forth for differentiation of mature beta cells in Example 6, auxotrophic selection methods can be used to select for cells differentiated into defined subsets of cardiomyocytes, specifically ventricular cardiomyocytes.

TBX5 (ENSG00000089225) and NKX2-5 (ENSG00000183072) gene expression mark ventricular myocyte cells, and their differential expression identify at least four different lineage-specific subpopulations of human induced pluripotent stem cell-derived cardiomyocytes: TBX5-positive/NKX2-5-positive, TBX5-positive/NKX2-5-negative, TBX5-negative/NKX2-5-positive, and TBX5-negative/NKX2-5-negative. (See Zhang, Joe Z., et al. “A human iPSC double-reporter system enables purification of cardiac lineage subpopulations with distinct function and drug response profiles.” Cell stem cell 24.5 (2019): 802-811, the disclosure of which is incorporated by reference herein in its entirety). Specifically, TBX5-positive/NKX2-5-positive cells represent a lineage close to first heart field lineage cells useful in differentiating into ventricular cardiomyocytes; TBX5-positive/NKX2-5-negative cells represent an epicardial lineage useful in differentiating into nodal cardiomyocytes; TBX5-negative/NKX2-5-positive cells represent a subpopulation similar to second heart field lineage cells useful in differentiating into atrial cardiomyocytes; and TBX5-negative/NKX2-5-negative cells represent a subpopulation exhibiting endothelial cell properties.

It is now specifically contemplated that cardiomyocytes, including sub-populations of cardiomyocytes derived from human iPSCs. Specifically, epicardial lineage cells useful in differentiating into nodal cardiomyocytes can be specifically selected for from a population of in vitro differentiated human iPSCs by starting with UMPS/uridine auxotrophic human iPSCs and inserting transgenes expressing, for example, TBX5 or a TBX5-dependent transgene, wherein the transgene is regulated by endogenous TBX5 expression control sequences and further comprises UMPS. Insertion of the TBX5-UMPS transgene under control of endogenous TBX5 expression control sequences into the uridine-auxotrophic cells enables re-expression of UMPS only in cells that express TBX5.

A sub-population similar to second heart field lineage cells useful in differentiating into atrial cardiomyocytes can be specifically selected for from a population of in vitro differentiated human iPSCs by starting with UMPS/uridine auxotrophic human iPSCs and inserting transgenes expressing, for example, NKX2-5 or a NKX2-5-dependent transgene, wherein the transgene is regulated by endogenous NKX2-5 expression control sequences and further comprises UMPS. Insertion of the NKX2-5-UMPS transgene under control of endogenous NKX2-5expression control sequences into the uridine-auxotrophic cells enables re-expression of UMPS only in cells that express NKX2-5.

Dual-specific selection methods using split auxotrophy can be used to select for a sub-population of cells that differentiate into ventricular myocytes. For example, ventricular myocytes can be specifically selected for from a population of in vitro differentiated human cells by, for example, starting with UMPS/uridine auxotrophic human iPSCs and inserting a first transgene expressing, for example, TBX5 or an TBX5-dependent transgene, wherein the transgene is regulated by endogenous TBX5 expression control sequences and further comprises a first UMPS independent functional domain (e.g., ODC). Cells expressing only ODC will remain auxotrophic for uridine. Therefore, a second transgene expressing, for example, OPRT, must be expressed to relieve auxotrophy and permit withdrawal of uridine. The expression of the second transgene can be under regulation of endogenous expression control sequences native to a second ventricular cardiomyocyte cell-specific factor, e.g., NKX2-5. Thus, upon withdrawal of the auxotrophic factor uridine from the population of in vitro differentiated cells, only those cells expressing both TBX5 and NKX2-5 will survive, thereby selecting for ventricular myocytes.

### Example 9. Generation of Stable T Reg Cell Populations

Stable T reg cell populations can be generated using the selection methods employing the split auxotrophic systems described herein. Passerini et al have shown that conventional CD4+ T cells can be converted into fully functional T reg-like cells by introducing FOXP3 expression. Moreover, it has been shown that stable expression of FOXP3 in CD4+T regs indicates stable, as opposed to plastic, T reg cells. (See Passerini, Laura, et al. “CD4+ T cells from IPEX patients convert into functional and stable regulatory T cells by FOXP3 gene transfer.” Science translational medicine 5.215 (2013): 215ra174-215ra174, the disclosure of which is incorporated by reference herein in its entirety.)

It is now specifically contemplated that a first independent functional domain of UMPS (e.g., ODC) can be expressed under control of an expression control sequence of FOXP3 (ENSG00000049768), for example using the FOXP3 promoter, and a second independent functional domain of UMPS (e.g., OPRT) can be expressed under control of an expression control sequence of a cell naïveté-associated promoter (e.g., a protein tyrosine phosphatase receptor type C (PTPRC) [ENSG00000081237]: CD45RA or CD45RO; or CCR7 [ENSG00000126353]). Cells incorporating both the FOXP3 promoter-ODC and OPRT under control of a naïveté-associated promoter will represent stable T reg cells.

Split auxotrophic selection methods can also be used to select for and stabilize CAR T cell lines and to produce allogeneic cells. For example, T cells can be isolated and engineered to be auxotrophic by interrupting the UMPS gene. A first homologous recombination donor vector targeting the UMPS locus can be engineered to knock out endogenous UMPS expression and knock in a first independent functional domain of UMPS, e.g., OPRT. The first homologous recombination donor vector can include a FOXP3 coding sequence operably linked to the first independent functional domain of UMPS. The FOXP3 coding sequence and the sequence encoding the first independent functional domain of UMPS can be operably linked, for example, by an IRES sequence. The isolated T cells can further be engineered with a second homologous recombination donor vector targeting, e.g., the T cell receptor (TCR) alpha constant (TRAC) locus, encoding endogenous TCR alpha (TCRA). The second homologous recombination donor vector can include a sequence encoding a chimeric antigen receptor (CAR) and a sequence encoding the second independent functional domain of UMPS, e.g., ODC. The CAR coding sequence and the sequence encoding the second independent functional domain of UMPS can be operably linked, for example, by an IRES sequence. Double knock-in cells will functionally re-express UMPS, and will survive in the absence of uridine, whereas single knock-in or non-knock-in cells will starve or fail to proliferate in the absence of uridine. Thus, the split auxotrophy system ensures only CAR expressing, endogenous TCR knockout, FOXP3-positive cells survive and/or proliferate.

### Example 10. Split Auxotrophic Selection for Optimizing Expression Vectors for Use in PS Cell-Derived Engineered Megakaryocytes

Progenitor cells such as induced pluripotent stem cells are engineered according to the methods described herein to have uridine auxotrophy by generating UMPS knockout cells and selecting for knockout cells by culturing in uridine-containing medium according to the methods described herein (see, e.g., Example 1).

UMPS knockout cells are transfected with a first and a second homologous recombination donor vector. The first homologous recombination donor vector carries: 1) OPRT coding sequence (SEQ ID NO: 4, encoding amino acid sequence of SEQ ID NO: 5) under transcriptional regulation of an expression control sequence comprising a constitutive promoter such as EF1a and 2) a nucleotide sequence encoding a first payload under transcriptional regulation of an expression control sequence comprising of a megakaryocyte-specific promoter such as PF4. The second homologous recombination donor vector carries: 1) ODC (SEQ ID NO: 6, encoding amino acid sequence of SEQ ID NO: 7) under transcriptional regulation of an expression control sequence of a constitutive promoter such as EF1a and 2) a nucleotide sequence encoding a second payload under transcriptional regulation of an expression control sequence of a megakaryocyte-specific promoter such as PF4. First and second homologous recombination vectors can have homology arms targeting a safe harbor locus such as CCR5 (using, for example, CCR5 left and right homology arms selected from SEQ ID NOs: 11-15 as described herein).

Transfected cells are cultured in the absence of uridine. UMPS knockout cells successfully transfected with both first and second homologous recombination donor vectors survive uridine withdrawal, while cells not successfully expressing both first and second homologous recombination donor vectors die, thereby selecting for double knock-in cells expressing both independent functional domains of UMPS.

Double knock-in cells are assessed for expression and function of payload(s) using methods known in the art. Expression levels can be optimized by adjusting promoter or coding sequence, by incorporating linker, or other transcriptional regulatory sequences. Double knock-in cells expressing desired levels of payload are identified as having optimized first and second homologous recombination donor vectors. Optimized first and second homologous recombination donor vectors are subsequently used to design optimized first and second vectors lacking UMPS independent functional domains. That is, an optimized first vector includes a nucleotide sequence encoding a first payload under transcriptional regulation of an expression control sequence of a megakaryocyte-specific promoter such as PF4, and lacks a UMPS independent functional domain coding sequence/promoter; and an optimized second vector includes a nucleotide sequence encoding a second payload under transcriptional regulation of an expression control sequence of a megakaryocyte-specific promoter such as PF4, and lacks a UMPS independent functional domain coding sequence/promoter. Optimized first and second vectors include homologous recombination arms targeting a safe harbor locus such as CCR5 (using, for example, CCR5 left and right homology arms selected from SEQ ID NOs: 11-15 as described herein).

The optimized first and second vectors are transfected into UMPS knockout cells cultured in the presence of uridine. Clones expressing both first and second optimized vectors are selected. Select clonal populations can be differentiated into, e.g., megakaryocytes and/or further into platelets produced from megakaryocytes, whereupon megakaryocyte-specific promoters drive expression of payload(s). Megakaryocytes and/or engineered platelets described herein may be produced using a technique described in Moreau, Thomas, et al. “Large-scale production of megakaryocytes from human pluripotent stem cells by chemically defined forward programming.” Nature communications 7 (2016): 11208; Ito, Yukitaka, et al. “Turbulence activates platelet biogenesis to enable clinical scale ex vivo production.” Cell 174.3 (2018): 636-648; and/or Feng Q, et al. “Scalable generation of universal platelets from human induced pluripotent stem cells.” Stem cell reports. 2014; 3(5):817-831, each of which is hereby incorporated by reference in its entirety. These studies provide methods for generating immortalized megakaryocyte progenitor cell lines from iPSCs and clinical scale production of platelets.

### Example 11. Generation of Megakaryocytes

Progenitor cells such as induced pluripotent stem cells are engineered according to the methods described herein to have uridine auxotrophy by generating UMPS knockout cells and selecting for knockout cells by culturing in uridine-containing medium according to the methods described herein (see, e.g., Example 1).

UMPS knockout cells can be differentiated into megakaryocytes. Megakaryocytes and/or engineered platelets described herein may be produced using a technique described, for example, in Moreau et al, Ito et al, or Feng Q, et al. Upon differentiation into megakaryocytes, uridine is withdrawn, whereupon proliferative cells including residual megakaryocytes die, while platelets produced from megakaryocytes persist due to a reduced requirement for uridine metabolism. Thus, an enriched population of platelets is generated from, e.g., human pluripotent stem cells which can be used in in vivo applications. In vivo uridine levels are sufficiently low as to preclude UMPS knockout cells from surviving or proliferating.

### Example 12. Split Auxotrophs for Production of Engineered Platelets In Vitro

Progenitor cells such as induced pluripotent stem cells are engineered according to the methods described herein to have uridine auxotrophy by generating UMPS knockout cells and selecting for knockout cells by culturing in uridine-containing medium according to the methods described herein (see, e.g., Example 1).

UMPS knockout cells are transfected with a first and a second homologous recombination donor vector. The first homologous recombination donor vector carries: 1) OPRT coding sequence (SEQ ID NO: 4, encoding amino acid sequence of SEQ ID NO: 5) under transcriptional regulation of an expression control sequence of a constitutive promoter such as EF1a and 2) a nucleotide sequence encoding a first payload protein under transcriptional regulation of an expression control sequence of a megakaryocyte-specific promoter such as PF4. The second homologous recombination donor vector carries: 1) ODC (SEQ ID NO: 6, encoding amino acid sequence of SEQ ID NO: 7) under transcriptional regulation of an expression control sequence of a constitutive promoter such as EF1a and 2) a nucleotide sequence encoding a second payload protein under transcriptional regulation of an expression control sequence of a megakaryocyte-specific promoter such as PF4. First and second homologous recombination vectors can have homology arms targeting a safe harbor locus such as CCR5 (using, for example, CCR5 left and right homology arms selected from SEQ ID NOs: 11-15 as described herein).

Transfected cells are cultured in the absence of uridine. UMPS knockout cells successfully transfected with both first and second homologous recombination donor vectors survive uridine withdrawal, while cells not successfully expressing both first and second homologous recombination donor vectors die, thereby selecting for double knock-in cells expressing both independent functional domains of UMPS.

Double knock-in cells are differentiated in vitro to megakaryocytes using a technique described, for example, in Moreau et al, Ito, Y., et al, or Feng, Q., et al. Differentiated cells stop expressing EF1a-driven OPRT/ODC independent functional domains of UMPS. 5-FOA selection is used to eliminate any residual pluripotent cells. Remaining megakaryocytes produce platelets that can be used in downstream therapeutic applications. Uridine is withdrawn and any remaining nucleated, proliferating megakaryocytes or other proliferating cells die, leaving a pure population of platelets derived from progenitor cells in vitro.

### EQUIVALENTS AND SCOPE

Those skilled in the art will recognize, or be able to ascertain using no more than routine experimentation, many equivalents to the specific embodiments in accordance with the present disclosure. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the present disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the present disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present disclosure.

What is claimed is:

1. A method of generating a population of differentiated cells comprising:
(a) contacting a plurality of progenitor cells with a CRISPR/Cas system comprising a guide RNA (gRNA) targeting an inessential portion of a promoter of a gene;
(b) inserting biallelically by homologous recombination a construct comprising a tissue-specific promoter and at least a portion of the gene, wherein the gene is selected from the group consisting of: AACS, AADAT, AASDHPPT, AASS, ACAT1, ACCS, ACCSL, ACO1, ACO2, ACSS3, ADSL, ADSS, ADSSL1, ALAD, ALAS1, ALAS2, ALDH1A1, ALDH1A2, ALDH1A3, ALDH1B1, ALDH2, AMD1, ASL, ASS1, ATF4, ATF5, AZIN1, AZIN2, BCAT1, BCAT2, CAD, CBS, CBSL, CCBL1, CCBL2, CCS, CEBPA, CEBPB, CEBPD, CEBPE, CEBPG, CH25H, COQ6, CPS1, CTH, CYP51A1, DECR1, DHFR, DHFRL1, DHODH, DHRS7, DHRS7B, DHRS7C, DPYD, DUT, ETFDH, FAXDC2, FDFT1, FDPS, FDXR, FH, FPGS, G6PD, GCAT, GCH1, GCLC, GFPT1, GFPT2, GLRX5, GLUL, GMPS, GPT, GPT2, GSX2, H6PD, HAAO, HLCS, HMBS, HMGCL, HMGCLL1, HMGCS1, HMGCS2, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HRSP12, HSD11B1, HSD11B1L, HSD17B12, HSD17B3, HSD17B7, HSD17B7P2, HSDL1, HSDL2, IBA57, IDO1, IDO2, IL4I1, ILVBL, IP6K1, IP6K2, IP6K3, IPMK, IREB2, ISCA1, ISCA1P1, ISCA2, KATNA1, KATNAL1, KATNAL2, KDM1B, KDSR, KMO, KYNU, LGSN, LSS, MARS, MARS2, MAX, MITF, MLX, MMS19, MPC1, MPC1L, MPI, MSMO1, MTHFD1, MTHFD1L, MTHFD2, MTHFD2L, MTHFR, MTRR, MVK, MYB, MYBL1, MYBL2, NAGS, ODC1, OTC, PAICS, PAOX, PAPSS1, PAPSS2, PDHB, PDX1, PFAS, PIN1, PLCB1, PLCB2, PLCB3, PLCB4, PLCD1, PLCD3, PLCD4, PLCE1, PLCG1, PLCG2, PLCH1, PLCH2, PLCL1, PLCL2, PLCZ1, PM20D1, PPAT, PSAT1, PSPH, PYCR1, PYCR2, QPRT, RDH8, RPUSD2, SCD, SCD5, SLC25A19, SLC25A26, SLC25A34, SLC25A35, SLC7A10, SLC7A11, SLC7A13, SLC7A5, SLC7A6, SLC7A7, SLC7A8, SLC7A9, SMOX, SMS, SNAPC4, SOD1, SOD3, SQLE, SRM, TAT, TFE3, TFEB, TFEC, THNSL1, THNSL2, TKT, TKTL1, TKTL2, UMPS, UROD, UROS, USF1, USF2, VPS33A, VPS33B, VPS36, VPS4A, and VPS4B, resulting in the progenitor cells being auxotrophic for an auxotrophic factor;
(c) contacting the plurality of progenitor cells with the auxotrophic factor;
(d) stimulating differentiation of the progenitor cells into a tissue associated with the tissue-specific promoter, wherein the gene is expressed in response to differentiation; and
(e) removing the auxotrophic factor, thereby selecting for differentiated cells to generate the population of differentiated cells.

2. The method of claim 1, further comprising contacting the plurality of progenitor cells with 5-FOA.

3. The method of claim 1 or 2, wherein the gene is a UMPS gene.

4. The method of claim 3, wherein the tissue-specific promoter replaces the promoter of the UMPS gene.

5. The method of any one of claims 1-4, wherein the auxotrophic factor is uracil or a source of uracil.

6. The method of any one of claims 1-5, wherein the construct further comprises a nucleotide sequence encoding a therapeutic factor which is expressed in response to differentiation.

7. The method of claim 6, further comprising expressing the therapeutic factor as a cassette with the at least a portion of the UMPS gene.

8. The method of any one of claims 1-7, wherein the construct is polycistronic.

9. The method of claim 8, wherein the construct comprises an internal ribosome entry site (IRES) or a peptide 2A sequence (P2A).

10. The method of any one of claims 3-9, wherein the at least a portion of the UMPS gene is a homology arm.

11. The method of any one of claims 1-10, wherein the plurality of progenitor cells is selected from the group consisting of hematopoietic stem cells (HSCs), embryonic stem cells, transdifferentiated stem cells, neural progenitor cells, mesenchymal stem cells, osteoblasts, cardiomyocytes, and combinations thereof.

12. The method of any one of claims 1-11, wherein the tissue is selected from the group consisting of adipose tissue, adrenal gland, ascites, bladder, blood, bone, bone marrow, brain, cervix, connective tissue, ear, embryonic tissue, esophagus, eye, heart, hematopoietic tissue, intestine, kidney, larynx, liver, lung, lymph, lymph node, mammary gland, mouth, muscle, nerve, ovary, pancreas, parathyroid, pharynx, pituitary gland, placenta, prostate, salivary gland, skin, spleen, stomach, testis, thymus, thyroid, tonsil, trachea, umbilical cord, uterus, endocrine, neuronal tissue, and vascular tissue.

13. The method of any one of claims 1-12, wherein the the population of differentiated cells comprises immune cells.

14. The method of claim 13, wherein the immune cells are selected from the group consisting of T cells, B cells, natural killer (NK) cells, and combinations thereof.

15. The method of any one of claims 1-14, wherein the tissue-specific promoter is selected from the group consisting of WAS proximal promoter; CD4 mini-promoter/enhancer; CD2 locus control region; CD4 minimal promoter and proximal enhancer and silencer; CD4 mini-promoter/enhancer; GATA-1 enhancer HS2 within the LTR; Ankyrin-1 and α-spectrin promoters combined or not with HS-40, GATA-1, ARE and intron 8 enhancers; Ankyrin-1 promoter/β-globin HS-40 enhancer; GATA-1 enhancer HS1 to HS2 within the retroviral LTR; Hybrid cytomegalovirus (CMV) enhancer/β-actin promoter; MCH II-specific HLA-DR promoter; Fascin promoter (pFascin); Dectin-2 gene promoter; 5′ untranslated region from the DC-STAMP; Heavy chain intronic enhancer (Ep) and matrix attachment regions; CD19 promoter; Hybrid immunoglobulin promoter (Igk promoter, intronic Enhancer and 3′ enhancer from Ig genes); CD68L promoter and first intron; Glycoprotein Ibα promoter; Apolipoprotein E (Apo E) enhancer/alpha1-antitrypsin (hAAT) promoter (ApoE/hAAT); HAAT promoter/Apo E locus control region; Albumin promoter; HAAT promoter/four copies of the Apo E enhancer; Albumin and hAAT promoters/al-microglobulin and prothrombin enhancers; HAAT promoter/Apo E locus control region; hAAT promoter/four copies of the Apo E enhancer; TBG promoter (thyroid hormone-binding globulin promoter and α1-microglobulin/bikunin enhancer); DC172 promoter (α1-antitrypsin promoter and α1-microglobulin enhancer); LCAT, kLSP-IVS, ApoE/hAAT and liver-fatty acid-binding protein promoters; RU486-responsive promoter; Creatine kinase promoter; Creatine kinase promoter; Synthetic muscle-specific promoter C5-12; Creatine kinase promoter; Hybrid enhancer/promoter regions of α-myosin and creatine kinase (MHCK7); Hybrid enhancer/promoter regions of α-myosin and creatine kinase; Synthetic muscle-specific promoter C5-12; Cardiac troponin-1 proximal promoter; E-selectin and KDR promoters; Prepro-endothelin-1 promoter; KDR promoter/hypoxia-responsive element; Flt-1 promoter; Flt-1 promoter; ICAM-2 promoter; Synthetic endothelial promoter; Endothelin-1 gene promoter; Amylase promoter; Insulin and human pdx-1 promoters; TRE-regulated insulin promoter; Enolase promoter; Enolase promoter; TRE-regulated synapsin promoter; Synapsin 1 promoter; PDGF-β promoter/CMV enhancer; PDGF-β, synapsin, tubulin-α and Ca2+/calmodulin-PK2 promoters combined with CMV enhancer; Phosphate-activated glutaminase and vesicular glutamate transporter-1 promoters; Glutamic acid decarboxylase-67 promoter; Tyrosine hydroxylase promoter; Neurofilament heavy gene promoter; Human red opsin promoter; Keratin-18 promoter; keratin-14 (K14) promoter; and Keratin-5 promoter.

16. The method of any one of claims 1-15, wherein the construct is tagged with a conditional destabilization domain or a conditional ribozyme switch.

17. A method of generating a population of differentiated cells comprising:
(a) contacting a plurality of progenitor cells with a DNA sequence encoding one or more progenitor cell-specific miRNA target sites, wherein the DNA sequence is knocked into an auxotrophy-inducing gene resulting in the progenitor cells being auxotrophic for an auxotrophic factor, and wherein a progenitor cell-specific miRNA that binds the miRNA target sites is expressed in the progenitor cells;
(b) contacting the plurality of progenitor cells with the auxotrophic factor;
(c) stimulating differentiation of the progenitor cells, wherein differentiation suppresses expression of the progenitor cell-specific miRNA and activates expression of the gene; and
(d) removing the auxotrophic factor, thereby selecting for differentiated cells to generate the population of differentiated cells.

18. The method of claim 17, further comprising contacting the plurality of progenitor cells with 5-FOA.

19. The method of claim 17 or 18, wherein the auxotrophy-inducing gene is selected from the group consisting of: AACS, AADAT, AASDHPPT, AASS, ACAT1, ACCS, ACCSL, ACO1, ACO2, ACSS3, ADSL, ADSS, ADSSL1, ALAD, ALAS1, ALAS2, ALDH1A1, ALDH1A2, ALDH1A3, ALDH1B1, ALDH2, AMD1, ASL, ASS1, ATF4, ATF5, AZIN1, AZIN2, BCAT1, BCAT2, CAD, CBS, CBSL, CCBL1, CCBL2, CCS, CEBPA, CEBPB, CEBPD, CEBPE, CEBPG, CH25H, COQ6, CPS1, CTH, CYP51A1, DECR1, DHFR, DHFRL1, DHODH, DHRS7, DHRS7B, DHRS7C, DPYD, DUT, ETFDH, FAXDC2, FDFT1, FDPS, FDXR, FH, FPGS, G6PD, GCAT, GCH1, GCLC, GFPT1, GFPT2, GLRX5, GLUL, GMPS, GPT, GPT2, GSX2, H6PD, HAAO, HLCS, HMBS, HMGCL, HMGCLL1, HMGCS1, HMGCS2, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HRSP12, HSD11B1, HSD11B1L, HSD17B12, HSD17B3, HSD17B7, HSD17B7P2, HSDL1, HSDL2, IBA57, IDO1, IDO2, IL4I1, ILVBL, IP6K1, IP6K2, IP6K3, IPMK, IREB2, ISCA1, ISCA1P1, ISCA2, KATNA1, KATNAL1, KATNAL2, KDM1B, KDSR, KMO, KYNU, LGSN, LSS, MARS, MARS2, MAX, MITF, MLX, MMS19, MPC1, MPC1L, MPI, MSMO1, MTHFD1, MTHFD1L, MTHFD2, MTHFD2L, MTHFR, MTRR, MVK, MYB, MYBL1, MYBL2, NAGS, ODC1, OTC, PAICS, PAOX, PAPSS1, PAPSS2, PDHB, PDX1, PFAS, PIN1, PLCB1, PLCB2, PLCB3, PLCB4, PLCD1, PLCD3, PLCD4, PLCE1, PLCG1, PLCG2, PLCH1, PLCH2, PLCL1, PLCL2, PLCZ1, PM20D1, PPAT, PSAT1, PSPH, PYCR1, PYCR2, QPRT, RDH8, RPUSD2, SCD, SCD5, SLC25A19, SLC25A26, SLC25A34, SLC25A35, SLC7A10, SLC7A11, SLC7A13, SLC7A5, SLC7A6, SLC7A7, SLC7A8, SLC7A9, SMOX, SMS, SNAPC4, SOD1, SOD3, SQLE, SRM, TAT, TFE3, TFEB, TFEC, THNSL1, THNSL2, TKT, TKTL1, TKTL2, UMPS, UROD, UROS, USF1, USF2, VPS33A, VPS33B, VPS36, VPS4A, and VPS4B.

20. The method of any one of claims 17-19, wherein the auxotrophy-inducing gene is uridine monophosphate synthetase (UMPS) and the one or more progenitor cell-specific miRNA target sites is present in a mRNA transcript transcribed from the UMPS gene.

21. The method of any one of claims 17-20, wherein the one or more progenitor cell-specific miRNA target sites is in the 3′ untranslated region (UTR) of a transcript transcribed from the auxotrophy-inducing gene.

22. The method of any one of claims 17-21, wherein the auxotrophic factor is uracil or a source of uracil.

23. The method of any one of claims 17-22, further comprising inserting into the genome of the progenitor cells a construct comprising a gene encoding a therapeutic factor, wherein expression of the therapeutic factor is controlled by the same promoter as the promoter controlling expression of the auxotrophy-inducing gene and the differentiated cells express the therapeutic factor.

24. The method of any one of claims 17-23, further comprising expressing the therapeutic factor as a cassette in-frame with the auxotrophy-inducing gene.

25. The method of claim 24, wherein the cassette comprises an internal ribosome entry site (IRES) or a peptide 2A sequence (P2A).

26. The method of any one of claims 20-25, wherein the DNA sequence encoding the one or more progenitor cell-specific miRNA target sites further comprises a homology arm targeting the auxotrophy-inducing gene.

27. The method of any one of claims 17-26, wherein the plurality of progenitor cells is selected from the group consisting of hematopoietic stem cells (HSCs), embryonic stem cells, transdifferentiated stem cells, neural progenitor cells, mesenchymal stem cells, osteoblasts, cardiomyocytes, and combinations thereof.

28. The method of any one of claims 17-27, wherein the stimulating differentiation of the progenitor cells produces differentiated cells of a cell or tissue type selected from the group consisting of adipose tissue, adrenal gland, ascites, bladder, blood, bone, bone marrow, brain, cervix, connective tissue, ear, embryonic tissue, esophagus, eye, heart, hematopoietic tissue, intestine, kidney, larynx, liver, lung, lymph, lymph node, mammary gland, mouth, muscle, nerve, ovary, pancreas, parathyroid, pharynx, pituitary gland, placenta, prostate, salivary gland, skin, spleen, stomach, testis, thymus, thyroid, tonsil, trachea, umbilical cord, uterus, endocrine, neuronal tissue, and vascular tissue.

29. The method of any one of claims 17-28, wherein the the population of differentiated cells comprises immune cells.

30. The method of claim 29, wherein the immune cells are selected from the group consisting of T cells, B cells, natural killer (NK) cells, and combinations thereof.

31. The method of any one of claims 17-30, wherein the gene is tagged with a conditional destabilization domain or a conditional ribozyme switch.

32. A method of treating a disease, disorder, or condition in a subject, the method comprising: administering to the subject the immune cells of any one of claims 13, 14, 29, and 30.

33. A method of alleviating auxotrophy by producing an auxotrophic factor upon differentiation, the method comprising:
(a) providing a plurality of auxotrophic progenitor cells which have been generated by knockout of an auxotrophy-inducing gene; and
(b) inserting a construct comprising an open reading frame of the auxotrophy-inducing gene into a tissue-specific gene locus, wherein expression of the tissue-specific gene is not disrupted, thereby producing the auxotrophic factor upon differentiation of the progenitor cells into the tissue associated with the tissue-specific gene locus.

34. The method of claim 33, wherein the progenitor cells are selected from induced pluripotent stem cells (iPSCs) or embryonic stem cells (ESCs).

35. The method of claim 33 or 34, wherein the gene is selected from the group consisting of: a gene selected from the group consisting of: AACS, AADAT, AASDHPPT, AASS, ACAT1, ACCS, ACCSL, ACO1, ACO2, ACSS3, ADSL, ADSS, ADSSL1, ALAD, ALAS1, ALAS2, ALDH1A1, ALDH1A2, ALDH1A3, ALDH1B1, ALDH2, AMD1, ASL, ASS1, ATF4, ATF5, AZIN1, AZIN2, BCAT1, BCAT2, CAD, CBS, CBSL, CCBL1, CCBL2, CCS, CEBPA, CEBPB, CEBPD, CEBPE, CEBPG, CH25H, COQ6, CPS1, CTH, CYP51A1, DECR1, DHFR, DHFRL1, DHODH, DHRS7, DHRS7B, DHRS7C, DPYD, DUT, ETFDH, FAXDC2, FDFT1, FDPS, FDXR, FH, FPGS, G6PD, GCAT, GCH1, GCLC, GFPT1, GFPT2, GLRX5, GLUL, GMPS, GPT, GPT2, GSX2, H6PD, HAAO, HLCS, HMBS, HMGCL, HMGCLL1, HMGCS1, HMGCS2, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HRSP12, HSD11B1, HSD11B1L, HSD17B12, HSD17B3, HSD17B7, HSD17B7P2, HSDL1, HSDL2, IBA57, IDO1, IDO2, IL4I1, ILVBL, IP6K1, IP6K2, IP6K3, IPMK, IREB2, ISCA1, ISCA1P1, ISCA2, KATNA1, KATNAL1, KATNAL2, KDM1B, KDSR, KMO, KYNU, LGSN, LSS, MARS, MARS2, MAX, MITF, MLX, MMS19, MPC1, MPC1L, MPI, MSMO1, MTHFD1, MTHFD1L, MTHFD2, MTHFD2L, MTHFR, MTRR, MVK, MYB, MYBL1, MYBL2, NAGS, ODC1, OTC, PAICS, PAOX, PAPSS1, PAPSS2, PDHB, PDX1, PFAS, PIN1, PLCB1, PLCB2, PLCB3, PLCB4, PLCD1, PLCD3, PLCD4, PLCE1, PLCG1, PLCG2, PLCH1, PLCH2, PLCL1, PLCL2, PLCZ1, PM20D1, PPAT, PSAT1, PSPH, PYCR1, PYCR2, QPRT, RDH8, RPUSD2, SCD, SCD5, SLC25A19, SLC25A26, SLC25A34, SLC25A35, SLC7A10, SLC7A11, SLC7A13, SLC7A5, SLC7A6, SLC7A7, SLC7A8, SLC7A9, SMOX, SMS, SNAPC4, SOD1, SOD3, SQLE, SRM, TAT, TFE3, TFEB, TFEC, THNSL1, THNSL2, TKT, TKTL1, TKTL2, UMPS, UROD, UROS, USF1, USF2, VPS33A, VPS33B, VPS36, VPS4A, and VPS4B.

36. The method of claim 35, wherein the gene is uridine monophosphate synthetase (UMPS).

37. The method of any one of claims 33-36, wherein the construct further comprises an internal ribosome entry site (IRES) or a peptide 2A sequence (P2A).

38. The method of any one of claims 33-37, wherein the tissue-specific gene locus is an insulin locus.

39. The method of any one of claims 33-38, further comprising differentiating the plurality of auxotrophic progenitor cells to immune cells.

40. The method of claim 39, wherein the immune cells are T cells, a B cells, or natural killer (NK) cells.

41. The method of any one of claims 33-40, wherein the tissue-specific gene is not replaced during the inserting step.

42. The method of claim 41, further comprising producing insulin upon differentiation of the progenitor cells.

43. The method of any one of claims 33-42, wherein the gene is tagged with a conditional destabilization domain or a conditional ribozyme switch.

44. A method of selecting cells with plasmid integration or episomal expression, the method comprising:
(a) providing a plurality of cells with a knockout of an auxotrophy-inducing gene resulting in an auxotrophy in the plurality of cells, wherein the plurality of cells with the auxotrophy is grown in a medium providing an auxotrophic factor to the plurality of cells;
(b) transfecting the plurality of cells with a delivery system selected from the group consisting of a plasmid, a lentivirus, an adeno-associated virus (AAV), and a nanoparticle, wherein the delivery system expresses the auxotrophic factor; and
(c) removing the medium, thereby selecting cells with plasmid integration or episomal expression.

45. The method of claim 44, wherein the delivery system expresses at least one transgene.

46. The method of claim 44 or 45, wherein the gene is selected from the group consisting of: AACS, AADAT, AASDHPPT, AASS, ACAT1, ACCS, ACCSL, ACO1, ACO2, ACSS3, ADSL, ADSS, ADSSL1, ALAD, ALAS1, ALAS2, ALDH1A1, ALDH1A2, ALDH1A3, ALDH1B1, ALDH2, AMD1, ASL, ASS1, ATF4, ATF5, AZIN1, AZIN2, BCAT1, BCAT2, CAD, CBS, CBSL, CCBL1, CCBL2, CCS, CEBPA, CEBPB, CEBPD, CEBPE, CEBPG, CH25H, COQ6, CPS1, CTH, CYP51A1, DECR1, DHFR, DHFRL1, DHODH, DHRS7, DHRS7B, DHRS7C, DPYD, DUT, ETFDH, FAXDC2, FDFT1, FDPS, FDXR, FH, FPGS, G6PD, GCAT, GCH1, GCLC, GFPT1, GFPT2, GLRX5, GLUL, GMPS, GPT, GPT2, GSX2, H6PD, HAAO, HLCS, HMBS, HMGCL, HMGCLL1, HMGCS1, HMGCS2, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HRSP12, HSD11B1, HSD11B1L, HSD17B12, HSD17B3, HSD17B7, HSD17B7P2, HSDL1, HSDL2, IBA57, IDO1, IDO2, IL4I1, ILVBL, IP6K1, IP6K2, IP6K3, IPMK, IREB2, ISCA1, ISCA1P1, ISCA2, KATNA1, KATNAL1, KATNAL2, KDM1B, KDSR, KMO, KYNU, LGSN, LSS, MARS, MARS2, MAX, MITF, MLX, MMS19, MPC1, MPC1L, MPI, MSMO1, MTHFD1, MTHFD1L, MTHFD2, MTHFD2L, MTHFR, MTRR, MVK, MYB, MYBL1, MYBL2, NAGS, ODC1, OTC, PAICS, PAOX, PAPSS1, PAPSS2, PDHB, PDX1, PFAS, PIN1, PLCB1, PLCB2, PLCB3, PLCB4, PLCD1, PLCD3, PLCD4, PLCE1, PLCG1, PLCG2, PLCH1, PLCH2, PLCL1, PLCL2, PLCZ1, PM20D1, PPAT, PSAT1, PSPH, PYCR1, PYCR2, QPRT, RDH8, RPUSD2, SCD, SCD5, SLC25A19, SLC25A26, SLC25A34, SLC25A35, SLC7A10, SLC7A11, SLC7A13, SLC7A5, SLC7A6, SLC7A7, SLC7A8, SLC7A9, SMOX, SMS, SNAPC4, SOD1, SOD3, SQLE, SRM, TAT, TFE3, TFEB, TFEC, THNSL1, THNSL2, TKT, TKTL1, TKTL2, UMPS, UROD, UROS, USF1, USF2, VPS33A, VPS33B, VPS36, VPS4A, and VPS4B.

47. The method of any one of claims 44-46, wherein the gene is tagged with a conditional destabilization domain or a conditional ribozyme switch.

48. A kit comprising the materials for performing the method of any one of claims 1-47.

49. A method of generating a population of differentiated cells comprising:
(a) contacting a plurality of progenitor cells with a CRISPR/Cas system comprising a guide RNA (gRNA) targeting biallelically a portion of an auxotrophy-inducing gene, the auxotrophy-inducing gene comprising at least a first and a second independent functional domain, resulting in the progenitor cells being auxotrophic for an auxotrophic factor;
(b) contacting the plurality of progenitor cells with a first homologous recombination construct and a second homologous recombination construct, the first homologous recombination construct comprising a first tissue-specific promoter and at least a portion of the first independent functional domain of the auxotrophy-inducing gene, and the second homologous recombination construct comprising a second tissue-specific promoter and at least a portion of the second independent functional domain of the auxotrophy-inducing gene;
(c) contacting the plurality of progenitor cells with the auxotrophic factor;
(d) stimulating differentiation of the progenitor cells into a cell type or tissue expressing the first and the second tissue-specific promoters, wherein the first and the second homologous recombination constructs are expressed in differentiated cells; and
(e) selecting for differentiated cells by removing the auxotrophic factor, thereby generating the population of differentiated cells.

50. The method of claim 49, wherein the auxotrophy-inducing gene further comprises:
a third independent functional domain; a third and a fourth independent functional domain; or a third, a fourth, and a fifth independent functional domain;
the method further comprising contacting the plurality of progenitor cells with, respectively: a third homologous recombination construct comprising a third tissue-specific promoter and at least a portion of the third independent functional domain; a third and a fourth homologous recombination construct comprising a third and a fourth tissue-specific promoter and at least a portion of the third and the fourth independent functional domain; or a third, a fourth, and a fifth homologous recombination construct comprising a third, a fourth, and a fifth tissue-specific promoter and at least a portion of the third, the fourth, and the fifth independent functional domain;
wherein the cell type or tissue expresses the third, the third and the fourth, or the third, the fourth, and fifth tissue-specific promoters, respectively, and the third, the third and the fourth, or the third, the fourth, and the fifth homologous recombination constructs are all expressed in differentiated cells.

51. The method of claim 49, wherein the auxotrophy-inducing gene is uridine monophosphate synthase (UMPS), the first independent functional domain comprises orotate phosphoribosyltransferase (OPRT), and the second independent functional domain comprises orotidine 5′-phosphate decarboxylase (ODC).

52. The method of claim 50, wherein the auxotrophy-inducing gene is carbamoyl-phosphate synthetase 2, aspartate transcarbamylase, and dihydroorotase (CAD), the first independent functional domain comprises carbamoyl-phosphate synthetase 2, the second independent functional domain comprises aspartate transcarbamylase, and the third independent functional domain comprises dihydroorotase.

53. The method of any one of claims 49-52, further comprising contacting the progenitor cells with 5-FOA.

54. The method of any one of claims 49-53, wherein one or more of the homologous recombination constructs is inserted into a safe harbor locus.

55. The method of claim 54, wherein the safe harbor locus is CCR5.

56. The method of any one of claims 49-55, wherein the auxotrophic factor is uridine.

57. The method of any one of claims 49-56, wherein one or more of the homologous recombination constructs further comprises a nucleotide sequence encoding a therapeutic factor.

58. The method of any one of claims 49-57, wherein one or more of the homologous recombination constructs is polycistronic.

59. The method of claim 58, wherein one or more of the polycistronic constructs comprises an internal ribosome entry site (IRES) or a peptide 2A sequence (P2A).

60. The method of any one of claims 49-59, wherein the plurality of progenitor cells is selected from the group consisting of hematopoietic stem cells (HSCs), embryonic stem cells, transdifferentiated stem cells, neural progenitor cells, mesenchymal stem cells, osteoblasts, cardiomyocytes, and combinations thereof.

61. The method of any one of claims 49-60, wherein the cell type or tissue is selected from the group consisting of adipose tissue, adrenal gland, ascites, bladder, blood, bone, bone marrow, brain, cervix, connective tissue, ear, embryonic tissue, esophagus, eye, heart, hematopoietic tissue, intestine, kidney, larynx, liver, lung, lymph, lymph node, mammary gland, mouth, muscle, nerve, ovary, pancreas, parathyroid, pharynx, pituitary gland, placenta, prostate, salivary gland, skin, spleen, stomach, testis, thymus, thyroid, tonsil, trachea, umbilical cord, uterus, endocrine, neuronal, and vascular.

62. The method of any one of claims 49-61, wherein the differentiated cells are immune cells.

63. The method of claim 62, wherein the immune cells are selected from the group consisting of T cells, B cells, natural killer (NK) cells, and combinations thereof.

64. The method of any one of claims 49-63, wherein the two or more tissue-specific promoters are selected from the group consisting of WAS proximal promoter; CD4 mini-promoter/enhancer; CD2 locus control region; CD4 minimal promoter and proximal enhancer and silencer; CD4 mini-promoter/enhancer; GATA-1 enhancer HS2 within the LTR; Ankyrin-1 and α-spectrin promoters combined or not with HS-40, GATA-1, ARE and intron 8 enhancers; Ankyrin-1 promoter/β-globin HS-40 enhancer; GATA-1 enhancer HS1 to HS2 within the retroviral LTR; Hybrid cytomegalovirus (CMV) enhancer/β-actin promoter; MCH II-specific HLA-DR promoter; Fascin promoter (pFascin); Dectin-2 gene promoter; 5′ untranslated region from the DC-STAMP; Heavy chain intronic enhancer (Ep) and matrix attachment regions; CD19 promoter; Hybrid immunoglobulin promoter (Igk promoter, intronic Enhancer and 3′ enhancer from Ig genes); CD68L promoter and first intron; Glycoprotein Ibα promoter; Apolipoprotein E (Apo E) enhancer/alpha1-antitrypsin (hAAT) promoter (ApoE/hAAT); HAAT promoter/Apo E locus control region; Albumin promoter; HAAT promoter/four copies of the Apo E enhancer; Albumin and hAAT promoters/al-microglobulin and prothrombin enhancers; HAAT promoter/Apo E locus control region; hAAT promoter/four copies of the Apo E enhancer; TBG promoter (thyroid hormone-binding globulin promoter and α1-microglobulin/bikunin enhancer); DC172 promoter (α1-antitrypsin promoter and α1-microglobulin enhancer); LCAT, kLSP-IVS, ApoE/hAAT and liver-fatty acid-binding protein promoters; RU486-responsive promoter; Creatine kinase promoter; Creatine kinase promoter; Synthetic muscle-specific promoter C5-12; Creatine kinase promoter; Hybrid enhancer/promoter regions of α-myosin and creatine kinase (MHCK7); Hybrid enhancer/promoter regions of α-myosin and creatine kinase; Synthetic muscle-specific promoter C5-12; Cardiac troponin-1 proximal promoter; E-selectin and KDR promoters; Prepro-endothelin-1 promoter; KDR promoter/hypoxia-responsive element; Flt-1 promoter; Flt-1 promoter; ICAM-2 promoter; Synthetic endothelial promoter; Endothelin-1 gene promoter; Amylase promoter; Insulin and human pdx-1 promoters; TRE-regulated insulin promoter; Enolase promoter; Enolase promoter; TRE-regulated synapsin promoter; Synapsin 1 promoter; PDGF-β promoter/CMV enhancer; PDGF-β, synapsin, tubulin-α and Ca2+/calmodulin-PK2 promoters combined with CMV enhancer; Phosphate-activated glutaminase and vesicular glutamate transporter-1 promoters; Glutamic acid decarboxylase-67 promoter; Tyrosine hydroxylase promoter; Neurofilament heavy gene promoter; Human red opsin promoter; Keratin-18 promoter; keratin-14 (K14) promoter; and Keratin-5 promoter.

65. The method of any one of claims 49-64, wherein one or more of the homologous recombination constructs further comprises a nucleotide sequence encoding a conditional destabilization domain or a conditional ribozyme switch.

66. A method of treating a disease, disorder, or condition in a subject, the method comprising: administering to the subject the immune cells of claim 62 or 63.

67. A method of alleviating auxotrophy comprising:
(a) providing a plurality of auxotrophic progenitor cells which have been generated by knockout or knockdown of an auxotrophy-inducing gene comprising at least a first and a second independent functional domain; and
(b) inserting into the genome of the auxotrophic progenitor cells a first construct comprising an open reading frame of the first independent functional domain into a first tissue-specific gene locus, and inserting a second construct comprising an open reading frame of the second independent functional domain into a second tissue-specific gene locus, wherein expression of the tissue-specific genes at the first and second loci is not disrupted, thereby alleviating the auxotrophy upon differentiation of the progenitor cells into a cell type or tissue expressing the first and the second tissue-specific genes at the first and second loci.

68. The method of claim 67, wherein the auxotrophy-inducing gene further comprises:
a third independent functional domain; a third and a fourth independent functional domain; or a third, a fourth, and a fifth independent functional domain;
the method further comprising inserting into the genome of the auxotrophic progenitor cells, respectively: a third construct comprising an open reading frame of the third independent functional domain; a third construct comprising an open reading frame of the third independent functional domain and a fourth construct comprising an open reading frame of the fourth independent functional domain; or a third construct comprising an open reading frame of the third independent functional domain, a fourth construct comprising an open reading frame of the fourth independent functional domain, and a fifth construct comprising an open reading frame of the fifth independent functional domain;
wherein the cell type or tissue expresses the third, the third and the fourth, or the third, the fourth, and fifth tissue-specific genes, respectively, and the third, the third and the fourth, or the third, the fourth, and the fifth constructs are all expressed in differentiated cells.

69. The method of claim 67 or 68, wherein the progenitor cells are induced pluripotent stem cells (iPSCs) or embryonic stem cells (ESCs).

70. The method of any one of claims 67-69, wherein the auxotrophy-inducing gene is uridine monophosphate synthase (UMPS), the first independent functional domain comprises orotate phosphoribosyltransferase (OPRT), and the second independent functional domain comprises orotidine 5′-phosphate decarboxylase (ODC).

71. The method of any one of claims 67-69, wherein the auxotrophy-inducing gene is carbamoyl-phosphate synthetase 2, aspartate transcarbamylase, and dihydroorotase (CAD), the first independent functional domain comprises carbamoyl-phosphate synthetase 2, the second independent functional domain comprises aspartate transcarbamylase, and the third independent functional domain comprises dihydroorotase.

72. The method of any one of claims 67-71, wherein one or more of the constructs further comprises an internal ribosome entry site (IRES) or a peptide 2A sequence (P2A).

73. The method of any one of claims 67-72, wherein the tissue-specific gene locus is an insulin locus.

74. The method of any one of claims 67-73, further comprising differentiating the plurality of auxotrophic progenitor cells to immune cells.

75. The method of claim 74, wherein the immune cells are T cells, B cells, or natural killer (NK) cells.

76. The method of any one of claims 67-75, wherein the tissue-specific genes are not replaced during the inserting step.

77. The method of claim 76, further comprising producing insulin upon differentiation of the progenitor cells.

78. The method of any one of claims 67-77, wherein one or more of the constructs comprises a nucleotide sequence encoding a conditional destabilization domain or a conditional ribozyme switch.

79. A method of selecting cells having functionally integrated at least a first exogenous gene and a second exogenous gene, the method comprising:
(a) providing a plurality of cells with a knockout or knockdown of an auxotrophy-inducing gene comprising at least a first and a second independent functional domain, resulting in auxotrophy for an auxotrophic factor in the plurality of cells;
(b) growing the plurality of cells in a medium providing the auxotrophic factor;
(c) transfecting the plurality of cells with a first delivery system comprising a nucleotide sequence encoding the first exogenous gene and a nucleotide sequence encoding the first independent functional domain and a second delivery system comprising a nucleotide sequence encoding the second exogenous gene and a nucleotide sequence encoding the second independent functional domain; and
(d) replacing the medium with a medium lacking the auxotrophic factor, thereby selecting cells that have functionally integrated the first and second exogenous genes.

80. The method of claim 79, wherein the auxotrophy-inducing gene further comprises:
a third independent functional domain; a third and a fourth independent functional domain; or a third, a fourth, and a fifth independent functional domain;
the method further comprising transfecting the plurality of cells with, respectively: a third delivery system comprising a nucleotide sequence encoding a third exogenous gene and a nucleotide sequence encoding the third independent functional domain; a third delivery system comprising a nucleotide sequence encoding a third exogenous gene and a nucleotide sequence encoding the third independent functional domain and a fourth delivery system comprising a nucleotide sequence encoding a fourth exogenous gene and a nucleotide sequence encoding the fourth independent functional domain; or a third delivery system comprising a nucleotide sequence encoding a third exogenous gene and a nucleotide sequence encoding the third independent functional domain, a fourth delivery system comprising a nucleotide sequence encoding a fourth exogenous gene and a nucleotide sequence encoding the fourth independent functional domain, and a fifth delivery system comprising a nucleotide sequence encoding a fifth exogenous gene and a nucleotide sequence encoding the fifth independent functional domain.

81. The method of claim 79 or 80, wherein one or more of the delivery systems comprises a plasmid, a lentivirus, an adeno-associated virus (AAV), or a nanoparticle.

82. The method of any one of claims 79-81, wherein the auxotrophy-inducing gene is uridine monophosphate synthase (UMPS), the first independent functional domain comprises orotate phosphoribosyltransferase (OPRT), and the second independent functional domain comprises orotidine 5′-phosphate decarboxylase (ODC).

83. The method of any one of claims 79-81, wherein the auxotrophy-inducing gene is carbamoyl-phosphate synthetase 2, aspartate transcarbamylase, and dihydroorotase (CAD), the first independent functional domain comprises carbamoyl-phosphate synthetase 2, the second independent functional domain comprises aspartate transcarbamylase, and the third independent functional domain comprises dihydroorotase.

84. The method of any one of claims 79-83, wherein one or more of the delivery systems comprises a nucleotide sequence encoding a conditional destabilization domain or a conditional ribozyme switch.

85. A method of generating a population of mature human beta cells comprising:
(a) contacting a plurality of progenitor cells with a CRISPR/Cas system comprising a gRNA targeting biallelically a portion of a human UMPS gene resulting in the progenitor cells being auxotrophic for uridine;
(b) contacting the plurality of progenitor cells with a first homologous recombination construct and a second homologous recombination construct, the first homologous recombination construct comprising a nucleotide sequence encoding insulin or an insulin-dependent expression control sequence operably linked to a first independent functional domain of UMPS, and the second homologous recombination construct comprising a nucleotide sequence encoding Nkx6.1 or an Nkx6.1-dependent expression control sequence operably linked to a second independent functional domain of UMPS, wherein the first and the second independent functional domains are selected from OPRT and ODC and are expressed only in progenitor cells expressing both insulin and Nkx6.1;
(c) contacting the plurality of progenitor cells with uridine;
(d) stimulating differentiation of the plurality of progenitor cells into mature beta cells; and
(e) selecting for mature beta cells expressing both insulin and Nkx6.1 by removing uridine, thereby generating the population of mature human beta cells.

86. A method of alleviating type 1 diabetes in a subject comprising: administering to the subject the mature human beta cells of claim 85.

87. A mature human beta cell selected from a population of in vitro differentiated progenitor cells, the mature human beta cell comprising a biallelic genetic modification of an auxotrophy-inducing gene resulting in auxotrophy for an auxotrophic factor and one or more transgenes re-expressing the auxotrophy-inducing gene or one or more independent functional domains of the auxotrophy-inducing gene.

88. The mature human beta cell of claim 87, wherein the auxotrophy-inducing gene is UMPS, the auxotrophic factor is uridine, the independent functional domains are selected from OPRT and ODC, and the one or more transgenes further comprise a nucleotide sequence encoding insulin or an insulin-dependent expression control sequence and a nucleotide sequence encoding Nkx6.1 or an Nkx6.1-dependent expression control sequence.

89. A method of generating a sub-population of human cardiomyocytes comprising:
(a) contacting a plurality of progenitor cells with a CRISPR/Cas system comprising a gRNA targeting biallelically a portion of a human UMPS gene resulting in the progenitor cells being auxotrophic for uridine;
(b) contacting the plurality of progenitor cells with a first homologous recombination construct and a second homologous recombination construct, the first homologous recombination construct comprising a nucleotide sequence encoding TBX5 or a TBX5-dependent expression control sequence operably linked to a first independent functional domain of UMPS, and the second homologous recombination construct comprising a nucleotide sequence encoding NKX2-5 or a NKX2-5-dependent expression control sequence operably linked to a second independent functional domain of UMPS, wherein the first and the second independent functional domains are selected from OPRT and ODC and are expressed only in progenitor cells expressing one or both of TBX5 and NKX2-5;
(c) contacting the plurality of progenitor cells with uridine;
(d) stimulating differentiation of the plurality of progenitor cells into cardiomyocytes; and
(e) selecting for a sub-population of cardiomyocytes expressing one or both of TBX5 and NKX2-5 by removing uridine, thereby generating the sub-population of human cardiomyocytes.

90. The method of claim 89, wherein cells expressing:
(a) both TBX5 and NKX2-5 represent a sub-population comprising ventricular cardiomyocytes;
(b) TBX5 but not NKX2-5 represent a sub-population comprising nodal cardiomyocytes;
(c) not TBX5 but NKX2-5 represent a sub-population comprising atrial cardiomyocytes; and
(d) neither TBX5 nor NKX2-5 represent endothelial cells.

91. A cardiomyocyte selected from a population of in vitro differentiated cardiomyocytes comprising a biallelic genetic modification of an auxotrophy-inducing gene resulting in auxotrophy for an auxotrophic factor and one or more transgenes re-expressing the auxotrophy-inducing gene or one or more independent functional domains of the auxotrophy-inducing gene.

92. The cardiomyocyte of claim 91, wherein the auxotrophy-inducing gene is UMPS, the auxotrophic factor is uridine, the independent functional domains are selected from OPRT and ODC, and the one or more transgenes further comprise a nucleotide sequence encoding TBX5 or a TBX5-dependent expression control sequence and a nucleotide sequence encoding NKX2-5 or a NKX2-5-dependent expression control sequence.

93. The cardiomyocyte of claim 91 or 92, wherein the cardiomyocyte belongs to a sub-population of cardiomyocytes selected from the group consisting of first heart field lineage cells, ventricular cardiomyocytes, epicardial lineage cells, nodal cardiomyocytes, second heart field lineage cells, and atrial cardiomyocytes.

94. Use of the cardiomyocyte of any one of claims 91-93 in a method of in vitro drug testing.

95. A method of generating a population of stable T reg cells comprising:
(a) contacting a plurality of progenitor cells with a CRISPR/Cas system comprising a gRNA targeting biallelically a portion of a human UMPS gene resulting in the progenitor cells being auxotrophic for uridine;
(b) contacting the plurality of progenitor cells with a first homologous recombination construct and a second homologous recombination construct, the first homologous recombination construct comprising a nucleotide sequence encoding FOXP3 or a FOXP3-dependent expression control sequence operably linked to a first independent functional domain of UMPS, and the second homologous recombination construct comprising a nucleotide sequence encoding a cell naïveté-associated promoter or an expression control sequence of a cell naïveté-associated promoter operably linked to a second independent functional domain of UMPS, wherein the first and the second independent functional domains are selected from OPRT and ODC and are expressed only in progenitor cells expressing both FOXP3 and a gene associated with the cell naïveté-associated promoter;
(c) contacting the plurality of progenitor cells with uridine;
(d) stimulating differentiation of the plurality of progenitor cells into stable T reg cells; and
(e) selecting for stable T reg cells expressing both FOXP3 and the gene associated with the cell naïveté-associated promoter by removing uridine, thereby generating the population of stable T reg cells.

96. The method of claim 95, wherein the cell naïveté-associated promoter is a promoter associated with PTPRC or CCR7.

97. A method of alleviating a disease, disorder, or condition in a subject comprising: administering to the subject the stable T reg cells produced by the method of claim 95 or 96, wherein the disease, disorder, or condition comprises an immune disease or cancer.

98. Use of the stable T reg cells produced by the method of claim 95 or 96 in a method for treating a disease, disorder, or condition in a subject, wherein the disease, disorder, or condition comprises an immune disease or cancer.

99. A population of stable T reg cells selected from a population T reg cells comprising a biallelic genetic modification of an auxotrophy-inducing gene resulting in auxotrophy for an auxotrophic factor and one or more transgenes re-expressing the auxotrophy-inducing gene or one or more independent functional domains of the auxotrophy-inducing gene.

100. The population of stable T reg cells of claim 99, wherein the auxotrophy-inducing gene is UMPS, the auxotrophic factor is uridine, the independent functional domains are selected from OPRT and ODC, and the one or more transgenes further comprise a nucleotide sequence encoding FOXP3 or a FOXP3-dependent expression control sequence and a nucleotide sequence encoding a cell naïveté-associated promoter or a gene associated with a cell naïveté-associated promoter, optionally wherein the cell naïveté-associated promoter is a promoter associated with PTPRC or CCR7.

101. A method of generating a population of cells having incorporated a first and a second expression cassette, the method comprising:
(a) culturing in the presence of uridine a plurality of cells genetically engineered to be auxotrophic for uridine;
(b) contacting the plurality of cells with a first expression construct and a second expression construct,
the first expression construct comprising a first expression cassette comprising a nucleotide sequence encoding a first payload and a second expression cassette comprising a nucleotide sequence encoding a first independent functional domain of UMPS, and
the second expression construct comprising a third expression cassette comprising a nucleotide sequence encoding a second payload and a fourth expression cassette comprising a nucleotide sequence encoding a second independent functional domain of UMPS; and
(c) withdrawing the uridine from the plurality of cells, thereby generating the population of cells having incorporated a first and a second expression cassette.

102. The method of claim 101, wherein the first expression construct is a homologous recombination construct targeting a specific genetic locus.

103. The method of claim 101 or 102, wherein the second expression construct is a homologous recombination construct targeting a specific genetic locus.

104. The method of claim 102 or 103, wherein the specific genetic locus is a safe harbor locus.

105. The method of claim 104, wherein the safe harbor locus is CCR5.

106. The method of any one of claims 101-105, wherein the plurality of cells genetically engineered to be auxotrophic for uridine comprises UMPS knockout cells.

107. The method of any one of claims 101-106, wherein the plurality of cells is derived from progenitor cells.

108. The method of any one of claims 101-107, wherein the nucleotide sequence encoding the first payload is under the transcriptional control of a tissue-specific promoter.

109. The method of any one of claims 101-107, wherein the nucleotide sequence encoding the second payload is under the transcriptional control of a tissue-specific promoter.

110. The method of any one of claims 101-109, wherein the nucleotide sequence encoding the first payload and the nucleotide sequence encoding the second payload are each under the transcriptional control of a tissue-specific promoter.

111. The method of any one of claims 101-110, wherein the nucleotide sequence encoding the first independent functional domain of UMPS is under the transcriptional control of a constitutive promoter.

112. The method of any one of claims 101-110, wherein the nucleotide sequence encoding the second independent functional domain of UMPS is under the transcriptional control of a constitutive promoter.

113. The method of any one of claims 101-112, wherein the nucleotide sequences encoding the first and the second independent functional domains of UMPS are each under the transcriptional control of a constitutive promoter.

114. The method of any one of claims 101-113, wherein the first and the second independent functional domains of UMPS are independently selected from OPRT and ODC.

115. The method of any one of claims 108-110, further comprising differentiating the cells in vitro to a desired cell type.

116. The method of claim 115, wherein the tissue-specific promoter is a megakaryocyte-specific promoter and the desired cell type is a megakaryocyte.

117. The method of claim 115 or 116, wherein differentiating the cells to the desired cell type leads to expression of the first payload, the second payload, or the first and the second payload.

118. A population of cells comprising a first and a second expression cassette generated by the method of any one of claims 101-116.

119. An engineered cell comprising a knockout of an auxotrophy-inducing gene and a first expression construct and a second expression construct, wherein the first expression construct and the second expression construct are stably integrated into the genome of the cell, and wherein the first expression construct and the second expression construct each comprises a nucleotide sequence encoding a first and a second independent functional domain of the auxotrophy-inducing gene.

120. The engineered cell of claim 119, wherein the first expression construct and the second expression construct are integrated into the genome of the cell by homologous recombination.

121. A method of generating megakaryocytes in vitro comprising:
(a) culturing in the presence of an auxotrophic factor a plurality of progenitor cells genetically engineered to be auxotrophic for the auxotrophic factor;
(b) differentiating the cells to megakaryocytes; and
(c) withdrawing the auxotrophic factor.

122. The method of claim 121, wherein the plurality of cells comprises progenitor cells.

123. The method of claim 121 or 122, wherein the plurality of cells comprises UMPS knockout cells.

124. The method of any one of claims 121-123, wherein the auxotrophic factor is uridine.

125. The method of any one of claims 121-124, wherein withdrawing the uridine causes proliferative cells to die or to fail to propagate.

126. The method of any one of claims 121-125, wherein the megakaryocytes generate platelets.

127. The method of any one of claims 121-126, wherein the platelets persist after withdrawing the auxotrophic factor.

128. The method of any one of claims 121-127, wherein a substantially pure population of platelets is generated.

129. A substantially pure population of platelets generated by the method of any one of claims 121-128.

130. A substantially pure population of platelets generated in vitro from a plurality of cells genetically engineered to be auxotrophic.

131. A method of generating a population of engineered platelets comprising:
(a) culturing in the presence of an auxotrophic factor a plurality of cells genetically engineered to be auxotrophic for the auxotrophic factor, the plurality of cells having a knockout of an auxotrophy-inducing gene;
(b) contacting the plurality of cells with a first expression construct and a second expression construct,
the first expression construct comprising a first expression cassette comprising a nucleotide sequence encoding a first payload and a second expression cassette comprising a nucleotide sequence encoding a first independent functional domain of the auxotrophy-inducing gene, and
the second expression construct comprising a third expression cassette comprising a nucleotide sequence encoding a second payload and a fourth expression cassette comprising a nucleotide sequence encoding a second independent functional domain of the auxotrophy-inducing gene; and
(c) withdrawing the uridine from the plurality of cells.

132. The method of claim 131, wherein the first expression, the second expression construct, or the first and the second expression construct is a homologous recombination construct targeting a specific genetic locus.

133. The method of claim 132, wherein the specific genetic locus is a safe harbor locus.

134. The method of claim 133, wherein the safe harbor locus is CCR5.

135. The method of any one of claims 131-134, wherein the auxotrophy-inducing gene is UMPS and the auxotrophic factor is uridine.

136. The method of claim 135, wherein the first and the second independent functional domains are selected from OPRT and ODC.

137. The method of any one of claims 131-136, wherein the plurality of cells is derived from progenitor cells.

138. The method of any one of claims 131-137, wherein the nucleotide sequence encoding the first payload is under the transcriptional control of a tissue-specific promoter.

139. The method of any one of claims 131-137, wherein the nucleotide sequence encoding the second payload is under the transcriptional control of a tissue-specific promoter.

140. The method of any one of claims 131-139, wherein the nucleotide sequence encoding the first payload and the nucleotide sequence encoding the second payload are each under the transcriptional control of a tissue-specific promoter.

141. The method of any one of claims 131-140, wherein the nucleotide sequence encoding the first independent functional domain is under the transcriptional control of a constitutive promoter.

142. The method of any one of claims 131-140, wherein the nucleotide sequence encoding the second independent functional domain is under the transcriptional control of a constitutive promoter.

143. The method of any one of claims 131-142, wherein the nucleotide sequences encoding the first and the second independent functional domains are each under the transcriptional control of a constitutive promoter.

144. The method of any one of claims 131-143, further comprising differentiating the cells in vitro to a desired cell type.

145. The method of claim 144, wherein the tissue-specific promoter is a megakaryocyte-specific promoter and the desired cell type is a megakaryocyte.

146. The method of any one of claim 144 or 145, wherein differentiating the cells to the desired cell type leads to expression of the first payload, the second payload, or the first and the second payload.

147. The method of claim 145 or 146, wherein the megakaryocytes produce platelets.

148. The method of claim 147, wherein the platelets are loaded with the first payload, the second payload, or the first and the second payload.

149. The method of any one of claims 144-148, wherein the differentiating the cells in vitro is in the presence of the auxotrophic factor.

150. The method of any one of claim 147, wherein the differentiated platelets do not express the first and the second independent functional domains.

151. The method of claim 150, further comprising adding 5-FOA.

152. The method of any one of claims 144-151, further comprising withdrawing the auxotrophic factor after differentiating the cells, wherein remaining nucleated, proliferating cells die or fail to propagate upon withdrawal of the auxotrophic factor.

153. An engineered cell comprising a knockout of UMPS, a first expression construct and a second expression construct, wherein the first expression construct and the second expression construct are stably integrated into the genome of the cell, and wherein the first expression construct and the second expression construct each comprises a nucleotide sequence encoding a first and a second independent functional domain of UMPS selected from OPRT and ODC.

154. The engineered cell of claim 153, wherein the first expression construct and the second expression construct are integrated into the genome of the cell by homologous recombination.

155. The engineered cell of claim 154, wherein the first expression construct and the second expression construct each comprises homology arms targeting to a specific genetic locus.

156. The engineered cell of claim 155, wherein the specific genetic locus is a safe harbor locus.

157. The engineered cell of claim 156, wherein the safe harbor locus is CCR5 and the homology arms are targeted to the CCR5 locus.

158. The engineered cell of any one of claims 153-157, wherein the first expression construct comprises an expression cassette further comprising a nucleotide sequence encoding a first payload.

159. The engineered cell of any one of claims 153-158, wherein the second expression construct comprises an expression cassette further comprising a nucleotide sequence encoding a second payload.

160. The engineered cell of claim 158 or 159, wherein the nucleotide sequence encoding the first payload comprises a nucleotide sequence encoding an antisense RNA, an siRNA, an aptamer, a microRNA mimic, an anti-miR, a synthetic mRNA, or a polypeptide.

161. The engineered cell of claim 160, comprising a first expression construct comprising a nucleotide sequence encoding a first payload and a second expression construct comprising a nucleotide sequence encoding a second payload.

162. The engineered cell of any one of claims 153-161, wherein the engineered cell is derived from or differentiated from a progenitor cell.

163. The engineered cell of claim 161, wherein the engineered cell is derived from or differentiated from a progenitor cell cultured in vitro.

164. The engineered cell of any one of claims 153-163 for use in a method of generating engineered platelets.

165. The engineered cell of any one of claims 160-163 for use in a method of generating engineered platelets.

166. The engineered cell of claim 165, wherein the engineered platelets are loaded with the first payload, the second payload, or the first and the second payload.

167. A substantially pure population of platelets prepared in vitro from cells engineered to be UMPS knockout cells.

168. The substantially pure population of platelets of claim 167, wherein the population of platelets is devoid or substantially devoid of nucleated or proliferative cells.

169. The substantially pure population of platelets of claim 167 or 168 for use in a method of treating a subject, the method comprising administering the platelets to the subject.

170. The substantially pure population of platelets of any one of claims 167-169 for use in a method of delivering a therapeutic payload to a subject in need thereof.


SEQUENCE LISTING (44 sequences)

SEQ ID NO: 1 - DNA, 1440 bp, Homo sapiens
atggcggtcgctcgtgcagctttggggccattggtgacgggtctgtacgacgtgcaggctttcaagtttggggacttcgtgctgaagagcgggctttcctcccccatctacatcgatctgcggggcatcgtgtctcgaccgcgtcttctgagtcaggttgcagatattttattccaaactgcccaaaatgcaggcatcagttttgacaccgtgtgtggagtgccttatacagctttgccattggctacagttatctgttcaaccaatcaaattccaatgcttattagaaggaaagaaacaaaggattatggaactaagcgtcttgtagaaggaactattaatccaggagaaacctgtttaatcattgaagatgttgtcaccagtggatctagtgttttggaaactgttgaggttcttcagaaggagggcttgaaggtcactgatgccatagtgctgttggacagagagcagggaggcaaggacaagttgcaggcgcacgggatccgcctccactcagtgtgtacattgtccaaaatgctggagattctcgagcagcagaaaaaagttgatgctgagacagttgggagagtgaagaggtttattcaggagaatgtctttgtggcagcgaatcataatggttctcccctttctataaaggaagcacccaaagaactcagcttcggtgcacgtgcagagctgcccaggatccacccagttgcatcgaagcttctcaggcttatgcaaaagaaggagaccaatctgtgtctatctgctgatgtttcactggccagagagctgttgcagctagcagatgctttaggacctagtatctgcatgctgaagactcatgtagatattttgaatgattttactctggatgtgatgaaggagttgataactctggcaaaatgccatgagttcttgatatttgaagaccggaagtttgcagatataggaaacacagtgaaaaagcagtatgaaggaggtatctttaaaatagcttcctgggcagatctagtaaatgctcacgtggtgccaggctcaggagttgtgaaaggcctgcaagaagtgggcctgcctttgcatcgggggtgcctccttattgcggaaatgagctccaccggctccctggccactggggactacactagagcagcggttagaatggctgaggagcactctgaatttgttgttggttttatttctggctcccgagtaagcatgaaaccagaatttcttcacttgactccaggagttcagttggaagcaggaggagataatcttggccaacagtacaatagcccacaagaagttattggcaaacgaggttccgatatcatcattgtaggtcgtggcataatctcagcagctgatcgtctggaagcagcagagatgtacagaaaagctgcttgggaagcgtatttgagtagacttggtgtt

SEQ ID NO: 2 - DNA, 1443 bp, Homo sapiens
atggcggtcgctcgtgcagctttggggccattggtgacgggtctgtacgacgtgcaggctttcaagtttggggacttcgtgctgaagagcgggctttcctcccccatctacatcgatctgcggggcatcgtgtctcgaccgcgtcttctgagtcaggttgcagatattttattccaaactgcccaaaatgcaggcatcagttttgacaccgtgtgtggagtgccttatacagctttgccattggctacagttatctgttcaaccaatcaaattccaatgcttattagaaggaaagaaacaaaggattatggaactaagcgtcttgtagaaggaactattaatccaggagaaacctgtttaatcattgaagatgttgtcaccagtggatctagtgttttggaaactgttgaggttcttcagaaggagggcttgaaggtcactgatgccatagtgctgttggacagagagcagggaggcaaggacaagttgcaggcgcacgggatccgcctccactcagtgtgtacattgtccaaaatgctggagattctcgagcagcagaaaaaagttgatgctgagacagttgggagagtgaagaggtttattcaggagaatgtctttgtggcagcgaatcataatggttctcccctttctataaaggaagcacccaaagaactcagcttcggtgcacgtgcagagctgcccaggatccacccagttgcatcgaagcttctcaggcttatgcaaaagaaggagaccaatctgtgtctatctgctgatgtttcactggccagagagctgttgcagctagcagatgctttaggacctagtatctgcatgctgaagactcatgtagatattttgaatgattttactctggatgtgatgaaggagttgataactctggcaaaatgccatgagttcttgatatttgaagaccggaagtttgcagatataggaaacacagtgaaaaagcagtatgaaggaggtatctttaaaatagcttcctgggcagatctagtaaatgctcacgtggtgccaggctcaggagttgtgaaaggcctgcaagaagtgggcctgcctttgcatcgggggtgcctccttattgcggaaatgagctccaccggctccctggccactggggactacactagagcagcggttagaatggctgaggagcactctgaatttgttgttggttttatttctggctcccgagtaagcatgaaaccagaatttcttcacttgactccaggagttcagttggaagcaggaggagataatcttggccaacagtacaatagcccacaagaagttattggcaaacgaggttccgatatcatcattgtaggtcgtggcataatctcagcagctgatcgtctggaagcagcagagatgtacagaaaagctgcttgggaagcgtatttgagtagacttggtgtttga

SEQ ID NO: 3 - protein, 480 aa, Homo sapiens
MAVARAALGPLVTGLYDVQAFKFGDFVLKSGLSSPIYIDLRGIVSRPRLLSQVADILFQTAQNAGISFDTVCGVPYTALPLATVICSTNQIPMLIRRKETKDYGTKRLVEGTINPGETCLIIEDVVTSGSSVLETVEVLQKEGLKVTDAIVLLDREQGGKDKLQAHGIRLHSVCTLSKMLEILEQQKKVDAETVGRVKRFIQENVFVAANHNGSPLSIKEAPKELSFGARAELPRIHPVASKLLRLMQKKETNLCLSADVSLARELLQLADALGPSICMLKTHVDILNDFTLDVMKELITLAKCHEFLIFEDRKFADIGNTVKKQYEGGIFKIASWADLVNAHVVPGSGVVKGLQEVGLPLHRGCLLIAEMSSTGSLATGDYTRAAVRMAEEHSEFVVGFISGSRVSMKPEFLHLTPGVQLEAGGDNLGQQYNSPQEVIGKRGSDIIIVGRGIISAADRLEAAEMYRKAAWEAYLSRLGV

SEQ ID NO: 4 - DNA, 642 bp, Homo sapiens
atggcggtcgctcgtgcagctttggggccattggtgacgggtctgtacgacgtgcaggctttcaagtttggggacttcgtgctgaagagcgggctttcctcccccatctacatcgatctgcggggcatcgtgtctcgaccgcgtcttctgagtcaggttgcagatattttattccaaactgcccaaaatgcaggcatcagttttgacaccgtgtgtggagtgccttatacagctttgccattggctacagttatctgttcaaccaatcaaattccaatgcttattagaaggaaagaaacaaaggattatggaactaagcgtcttgtagaaggaactattaatccaggagaaacctgtttaatcattgaagatgttgtcaccagtggatctagtgttttggaaactgttgaggttcttcagaaggagggcttgaaggtcactgatgccatagtgctgttggacagagagcagggaggcaaggacaagttgcaggcgcacgggatccgcctccactcagtgtgtacattgtccaaaatgctggagattctcgagcagcagaaaaaagttgatgctgagacagttgggagagtgaagaggtttattcaggagaatgtctttgtggcagcgaatcataatggttct

SEQ ID NO: 5 - protein, 214 aa, Homo sapiens
MAVARAALGPLVTGLYDVQAFKFGDFVLKSGLSSPIYIDLRGIVSRPRLLSQVADILFQTAQNAGISFDTVCGVPYTALPLATVICSTNQIPMLIRRKETKDYGTKRLVEGTINPGETCLIIEDVVTSGSSVLETVEVLQKEGLKVTDAIVLLDREQGGKDKLQAHGIRLHSVCTLSKMLEILEQQKKVDAETVGRVKRFIQENVFVAANHNGS

SEQ ID NO: 6 - DNA, 801 bp, Homo sapiens
cccctttctataaaggaagcacccaaagaactcagcttcggtgcacgtgcagagctgcccaggatccacccagttgcatcgaagcttctcaggcttatgcaaaagaaggagaccaatctgtgtctatctgctgatgtttcactggccagagagctgttgcagctagcagatgctttaggacctagtatctgcatgctgaagactcatgtagatattttgaatgattttactctggatgtgatgaaggagttgataactctggcaaaatgccatgagttcttgatatttgaagaccggaagtttgcagatataggaaacacagtgaaaaagcagtatgaaggaggtatctttaaaatagcttcctgggcagatctagtaaatgctcacgtggtgccaggctcaggagttgtgaaaggcctgcaagaagtgggcctgcctttgcatcgggggtgcctccttattgcggaaatgagctccaccggctccctggccactggggactacactagagcagcggttagaatggctgaggagcactctgaatttgttgttggttttatttctggctcccgagtaagcatgaaaccagaatttcttcacttgactccaggagttcagttggaagcaggaggagataatcttggccaacagtacaatagcccacaagaagttattggcaaacgaggttccgatatcatcattgtaggtcgtggcataatctcagcagctgatcgtctggaagcagcagagatgtacagaaaagctgcttgggaagcgtatttgagtagacttggtgtttaa

SEQ ID NO: 7 - protein, 266 aa, Homo sapiens
PLSIKEAPKELSFGARAELPRIHPVASKLLRLMQKKETNLCLSADVSLARELLQLADALGPSICMLKTHVDILNDFTLDVMKELITLAKCHEFLIFEDRKFADIGNTVKKQYEGGIFKIASWADLVNAHVVPGSGVVKGLQEVGLPLHRGCLLIAEMSSTGSLATGDYTRAAVRMAEEHSEFVVGFISGSRVSMKPEFLHLTPGVQLEAGGDNLGQQYNSPQEVIGKRGSDIIIVGRGIISAADRLEAAEMYRKAAWEAYLSRLGV

SEQ ID NO: 8 - RNA, 23 nt, Artificial Sequence
  Description of Artificial Sequence Synthetic Construct
ccccgcagaucgauguagauggg

SEQ ID NO: 9 - RNA, 23 nt, Artificial Sequence
  Description of Artificial Sequence Synthetic Construct
ggcggucgcucgugcagcuuugg

SEQ ID NO: 10 - RNA, 100 nt, Artificial Sequence
  Description of Artificial Sequence Synthetic Construct
gccccgcagaucgauguagaguuuuagagcuagaaauagcaaguuaaaauaaggcuaguccguuaucaacuugaaaaaguggcaccgagucggugcuuuu

SEQ ID NO: 11 - DNA, 802 bp, Artificial Sequence
  Description of Artificial Sequence Synthetic Construct
ttgtatctataaaaataaacaaaattagcttggtgtggtggcgcctgtagtccccagccacttggaggggtgaggtgagaggattgcttgagcccgggatgatccaggctgcagtgagccatgatcgtgccactgcactccagcctgggcgacagagtgagaccctgtctcacaacaacaacagcaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctctag

SEQ ID NO: 12 - DNA, 800 bp, Artificial Sequence
  Description of Artificial Sequence Synthetic Construct
tgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggctt

SEQ ID NO: 13 - DNA, 797 bp, Artificial Sequence
  Description of Artificial Sequence Synthetic Construct
ttgtatctataaaaataaacaaaattagcttggtgtggtggcgcctgtagtccccagccacttggaggggtgaggtgagaggattgcttgagcccgggatgatccaggctgcagtgagccatgatcgtgccactgcactccagcctgggcgacagagtgagaccctgtctcacaacaacaacagcaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttc

SEQ ID NO: 14 - DNA, 552 bp, Artificial Sequence
  Description of Artificial Sequence Synthetic Construct
cattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttc

SEQ ID NO: 15 - DNA, 541 bp, Artificial Sequence
  Description of Artificial Sequence Synthetic Construct
tgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcc

SEQ ID NO: 16 - DNA, 600 bp, Artificial Sequence
  Description of Artificial Sequence Synthetic Construct
tcgcccctcaaacaaatgccccgcagcccatttctccaccctcatttgatgaccgcagattcaagtgttttgttaagtaaagtcctgggtgacctggggtcacagggtgccccacgctgcctgcctctgggcgaacaccccatcacgcccggaggagggcgtggctgcctgcctgagtgggccagacccctgtcgccaggcctcacggcagctccatagtcaggagatggggaagatgctggggacaggccctggggagaagtactgggatcacctgttcaggctcccactgtgacgctgccccggggcgggggaaggaggtgggacatgtgggcgttggggcctgtaggtccacacccagtgtgggtgaccctccctctaacctgggtccagcccggctggagatgggtgggagtgcgacctagggctggcgggcaggcgggcactgtgtctccctgactgtgtcctcctgtgtccctctgcctcgccgctgttccggaacctgctctgcgcggcacgtcctggcagtggggcaggtggagctgggcgggggccctggtgcaggcagcctgcagcccttggccctggaggggtccctgc

SEQ ID NO: 17 - DNA, 74 bp, Artificial Sequence
  Description of Artificial Sequence Synthetic Construct
agaagagaggcatcgtggagcagtgttgcactagtatatgtagtctgtatcagttggaaaattattgtaactag

SEQ ID NO: 18 - DNA, 600 bp, Artificial Sequence
  Description of Artificial Sequence Synthetic Construct
agaagcgtggcattgtggaacaatgctgtaccagcatctgctccctctaccagctggagaactactgcaactagacgcagcccgcaggcagccccacacccgccgcctcctgcaccgagagagatggaataaagcccttgaaccagccctgctgtgccgtctgtgtgtcttgggggccctgggccaagccccacttcccggcactgttgtgagcccctcccagctctctccacgctctctgggtgcccacaggtgccaacgccggccaggcccagcatgcagtggctctccccaaagcggccatgcctgtcggctgcctgctgcccccaccctgtggctcagggtccagtatgggagctgcgggggtctctgaggggccaggggtggtggggccactgagaagtgacttcttgttcagtagctctggactcttggagtccccagagaccttgttcaggaaagggaatgagaacattccagcaattttccccccacctagccctcccaggttctatttttagagttatttctgatggagtccctgtggagggaggaggctgggctgagggagggggtcctgcagggcggggggctgggaag

SEQ ID NO: 19 - DNA, 54 bp, Artificial Sequence
  Description of Artificial Sequence Synthetic Construct
gagggcaggggcagcctgctgacctgcggcgacgtggaggagaaccccggcccc

SEQ ID NO: 20 - protein, 18 aa, Artificial Sequence
  Description of Artificial Sequence Synthetic Construct
EGRGSLLTCGDVEENPGP

SEQ ID NO: 21 - DNA, 84 bp, Artificial Sequence
  Description of Artificial Sequence Synthetic Construct
cgcaagcgccgcagcggcagcggcgagggccgcggcagcctgctgacctgcggcgacgtggaggagaaccccggccccaagctt

SEQ ID NO: 22 - protein, 28 aa, Artificial Sequence
  Description of Artificial Sequence Synthetic Construct
RKRRSGSGEGRGSLLTCGDVEENPGPKL

SEQ ID NO: 23 - DNA, 63 bp, Artificial Sequence
  Description of Artificial Sequence Synthetic Construct
ggaagcggagaaggcagaggaagcctgctcacctgcggcgatgttgaagaaaacccgggtcct

SEQ ID NO: 24 - protein, 21 aa, Artificial Sequence
  Description of Artificial Sequence Synthetic Construct
GSGEGRGSLLTCGDVEENPGP

SEQ ID NO: 25 - protein, 19 aa, Artificial Sequence
  Description of Artificial Sequence Synthetic Construct
ATNFSLLKQAGDVEENPGP

SEQ ID NO: 26 - protein, 22 aa, Artificial Sequence
  Description of Artificial Sequence Synthetic Construct
GSGATNFSLLKQAGDVEENPGP

SEQ ID NO: 27 - protein, 20 aa, Artificial Sequence
  Description of Artificial Sequence Synthetic Construct
QCTNYALLKLAGDVESNPGP

SEQ ID NO: 28 - protein, 23 aa, Artificial Sequence
  Description of Artificial Sequence Synthetic Construct
GSGQCTNYALLKLAGDVESNPGP

SEQ ID NO: 29 - protein, 22 aa, Artificial Sequence
  Description of Artificial Sequence Synthetic Construct
VKQTLNFDLLKLAGDVESNPGP

SEQ ID NO: 30 - protein, 25 aa, Artificial Sequence
  Description of Artificial Sequence Synthetic Construct
GSGVKQTLNFDLLKLAGDVESNPGP

SEQ ID NO: 31 - DNA, 1188 bp, Artificial Sequence
  Description of Artificial Sequence Synthetic Construct
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccgccacc

SEQ ID NO: 32 - DNA, 1193 bp, Artificial Sequence
  Description of Artificial Sequence Synthetic Construct
tctagggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccgccacc

SEQ ID NO: 33 - DNA, 596 bp, Artificial Sequence
  Description of Artificial Sequence Synthetic Construct
tccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaagaacacgatgataatatggccacaaccatgactagtgccacc

SEQ ID NO: 34 - DNA, 711 bp, Artificial Sequence
  Description of Artificial Sequence Synthetic Construct
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 35 - DNA, 708 bp, Artificial Sequence
  Description of Artificial Sequence Synthetic Construct
atggtatcaaagggtgaggaggacaatatggcgattataaaggagtttatgcgattcaaggtacacatggaaggatctgtgaacggacatgagtttgagatcgaaggcgagggggagggtcgaccttacgaaggaacacagacagcaaagctgaaggtcactaagggcggaccgctgcctttcgcctgggacatcctcagcccacagttcatgtacggttccaaggcatatgttaagcacccagcagatattccggactaccttaaacttagtttccctgaagggtttaaatgggaaagggtgatgaactttgaggatggaggcgtcgtaaccgttacacaggattcatctctgcaggatggcgagtttatctacaaagtaaaattgcggggtactaatttcccgagcgacggtcctgtaatgcagaaaaaaactatgggttgggaggccagctcagagcgaatgtacccagaagacggagccttgaagggggagattaaacagaggttgaagttgaaagacggcggtcattatgatgccgaagttaaaactacatacaaggcgaagaaacctgtccagcttccaggagcgtataatgtgaacataaaattggatattacgtcacataacgaggattacacaattgttgaacaatacgaaagggcggaggggaggcatagcacggggggtatggatgagctctataag

SEQ ID NO: 36 - protein, 236 aa, Artificial Sequence
  Description of Artificial Sequence Synthetic Construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 37 - DNA, 999 bp, Artificial Sequence
  Description of Artificial Sequence Synthetic Construct
atgcccccccccaggctgctgttcttcctgctgttcctgacccccatggaggtgaggcccgaggagcccctggtggtgaaggtggaggagggcgacaacgccgtgctgcagtgcctgaagggcaccagcgacggccccacccagcagctgacctggagcagggagagccccctgaagcccttcctgaagctgagcctgggcctgcccggcctgggcatccacatgaggcccctggccatctggctgttcatcttcaacgtgagccagcagatgggcggcttctacctgtgccagcccggcccccccagcgagaaggcctggcagcccggctggaccgtgaacgtggagggcagcggcgagctgttcaggtggaacgtgagcgacctgggcggcctgggctgcggcctgaagaacaggagcagcgagggccccagcagccccagcggcaagctgatgagccccaagctgtacgtgtgggccaaggacaggcccgagatctgggagggcgagcccccctgcctgccccccagggacagcctgaaccagagcctgagccaggacctgaccatggcccccggcagcaccctgtggctgagctgcggcgtgccccccgacagcgtgagcaggggccccctgagctggacccacgtgcaccccaagggccccaagagcctgctgagcctggagctgaaggacgacaggcccgccagggacatgtgggtgatggagaccggcctgctgctgcccagggccaccgcccaggacgccggcaagtactactgccacaggggcaacctgaccatgagcttccacctggagatcaccgccaggcccgtgctgtggcactggctgctgaggaccggcggctggaaggtgagcgccgtgaccctggcctacctgatcttctgcctgtgcagcctggtgggcatcctgcacctgcagagggccctggtgctgaggaggaagaggaagaggatgaccgaccccaccaggaggttc

SEQ ID NO: 38 - protein, 333 aa, Artificial Sequence
  Description of Artificial Sequence Synthetic Construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 39 - DNA, 255 bp, Artificial Sequence
  Description of Artificial Sequence Synthetic Construct
ctcgagggcgcgccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctagt

SEQ ID NO: 40 - DNA, 253 bp, Artificial Sequence
  Description of Artificial Sequence Synthetic Construct
aggatccaagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctttaattaacgaccgtacg

SEQ ID NO: 41 - DNA, 4337 bp, Artificial Sequence
  Description of Artificial Sequence Synthetic Construct
tcgcccctcaaacaaatgccccgcagcccatttctccaccctcatttgatgaccgcagattcaagtgttttgttaagtaaagtcctgggtgacctggggtcacagggtgccccacgctgcctgcctctgggcgaacaccccatcacgcccggaggagggcgtggctgcctgcctgagtgggccagacccctgtcgccaggcctcacggcagctccatagtcaggagatggggaagatgctggggacaggccctggggagaagtactgggatcacctgttcaggctcccactgtgacgctgccccggggcgggggaaggaggtgggacatgtgggcgttggggcctgtaggtccacacccagtgtgggtgaccctccctctaacctgggtccagcccggctggagatgggtgggagtgcgacctagggctggcgggcaggcgggcactgtgtctccctgactgtgtcctcctgtgtccctctgcctcgccgctgttccggaacctgctctgcgcggcacgtcctggcagtggggcaggtggagctgggcgggggccctggtgcaggcagcctgcagcccttggccctggaggggtccctgcagaagagaggcatcgtggagcagtgttgcactagtatatgtagtctgtatcagttggaaaattattgtaactagtccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaagaacacgatgataatatggccacaaccatgactagtgccaccatggtatcaaagggtgaggaggacaatatggcgattataaaggagtttatgcgattcaaggtacacatggaaggatctgtgaacggacatgagtttgagatcgaaggcgagggggagggtcgaccttacgaaggaacacagacagcaaagctgaaggtcactaagggcggaccgctgcctttcgcctgggacatcctcagcccacagttcatgtacggttccaaggcatatgttaagcacccagcagatattccggactaccttaaacttagtttccctgaagggtttaaatgggaaagggtgatgaactttgaggatggaggcgtcgtaaccgttacacaggattcatctctgcaggatggcgagtttatctacaaagtaaaattgcggggtactaatttcccgagcgacggtcctgtaatgcagaaaaaaactatgggttgggaggccagctcagagcgaatgtacccagaagacggagccttgaagggggagattaaacagaggttgaagttgaaagacggcggtcattatgatgccgaagttaaaactacatacaaggcgaagaaacctgtccagcttccaggagcgtataatgtgaacataaaattggatattacgtcacataacgaggattacacaattgttgaacaatacgaaagggcggaggggaggcatagcacggggggtatggatgagctctataagggaagcggagaaggcagaggaagcctgctcacctgcggcgatgttgaagaaaacccgggtcctatggcggtcgctcgtgcagctttggggccattggtgacgggtctgtacgacgtgcaggctttcaagtttggggacttcgtgctgaagagcgggctttcctcccccatctacatcgatctgcggggcatcgtgtctcgaccgcgtcttctgagtcaggttgcagatattttattccaaactgcccaaaatgcaggcatcagttttgacaccgtgtgtggagtgccttatacagctttgccattggctacagttatctgttcaaccaatcaaattccaatgcttattagaaggaaagaaacaaaggattatggaactaagcgtcttgtagaaggaactattaatccaggagaaacctgtttaatcattgaagatgttgtcaccagtggatctagtgttttggaaactgttgaggttcttcagaaggagggcttgaaggtcactgatgccatagtgctgttggacagagagcagggaggcaaggacaagttgcaggcgcacgggatccgcctccactcagtgtgtacattgtccaaaatgctggagattctcgagcagcagaaaaaagttgatgctgagacagttgggagagtgaagaggtttattcaggagaatgtctttgtggcagcgaatcataatggttctcccctttctataaaggaagcacccaaagaactcagcttcggtgcacgtgcagagctgcccaggatccacccagttgcatcgaagcttctcaggcttatgcaaaagaaggagaccaatctgtgtctatctgctgatgtttcactggccagagagctgttgcagctagcagatgctttaggacctagtatctgcatgctgaagactcatgtagatattttgaatgattttactctggatgtgatgaaggagttgataactctggcaaaatgccatgagttcttgatatttgaagaccggaagtttgcagatataggaaacacagtgaaaaagcagtatgaaggaggtatctttaaaatagcttcctgggcagatctagtaaatgctcacgtggtgccaggctcaggagttgtgaaaggcctgcaagaagtgggcctgcctttgcatcgggggtgcctccttattgcggaaatgagctccaccggctccctggccactggggactacactagagcagcggttagaatggctgaggagcactctgaatttgttgttggttttatttctggctcccgagtaagcatgaaaccagaatttcttcacttgactccaggagttcagttggaagcaggaggagataatcttggccaacagtacaatagcccacaagaagttattggcaaacgaggttccgatatcatcattgtaggtcgtggcataatctcagcagctgatcgtctggaagcagcagagatgtacagaaaagctgcttgggaagcgtatttgagtagacttggtgtttgaaggatccaagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctttaattaacgaccgtacgagaagcgtggcattgtggaacaatgctgtaccagcatctgctccctctaccagctggagaactactgcaactagacgcagcccgcaggcagccccacacccgccgcctcctgcaccgagagagatggaataaagcccttgaaccagccctgctgtgccgtctgtgtgtcttgggggccctgggccaagccccacttcccggcactgttgtgagcccctcccagctctctccacgctctctgggtgcccacaggtgccaacgccggccaggcccagcatgcagtggctctccccaaagcggccatgcctgtcggctgcctgctgcccccaccctgtggctcagggtccagtatgggagctgcgggggtctctgaggggccaggggtggtggggccactgagaagtgacttcttgttcagtagctctggactcttggagtccccagagaccttgttcaggaaagggaatgagaacattccagcaattttccccccacctagccctcccaggttctatttttagagttatttctgatggagtccctgtggagggaggaggctgggctgagggagggggtcctgcagggcggggggctgggaag

SEQ ID NO: 42 - DNA, 5250 bp, Srtificial Sequence
  Description of Artificial Sequence Synthetic Construct
ttgtatctataaaaataaacaaaattagcttggtgtggtggcgcctgtagtccccagccacttggaggggtgaggtgagaggattgcttgagcccgggatgatccaggctgcagtgagccatgatcgtgccactgcactccagcctgggcgacagagtgagaccctgtctcacaacaacaacagcaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctctagggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccgccaccatggcggtcgctcgtgcagctttggggccattggtgacgggtctgtacgacgtgcaggctttcaagtttggggacttcgtgctgaagagcgggctttcctcccccatctacatcgatctgcggggcatcgtgtctcgaccgcgtcttctgagtcaggttgcagatattttattccaaactgcccaaaatgcaggcatcagttttgacaccgtgtgtggagtgccttatacagctttgccattggctacagttatctgttcaaccaatcaaattccaatgcttattagaaggaaagaaacaaaggattatggaactaagcgtcttgtagaaggaactattaatccaggagaaacctgtttaatcattgaagatgttgtcaccagtggatctagtgttttggaaactgttgaggttcttcagaaggagggcttgaaggtcactgatgccatagtgctgttggacagagagcagggaggcaaggacaagttgcaggcgcacgggatccgcctccactcagtgtgtacattgtccaaaatgctggagattctcgagcagcagaaaaaagttgatgctgagacagttgggagagtgaagaggtttattcaggagaatgtctttgtggcagcgaatcataatggttctcccctttctataaaggaagcacccaaagaactcagcttcggtgcacgtgcagagctgcccaggatccacccagttgcatcgaagcttctcaggcttatgcaaaagaaggagaccaatctgtgtctatctgctgatgtttcactggccagagagctgttgcagctagcagatgctttaggacctagtatctgcatgctgaagactcatgtagatattttgaatgattttactctggatgtgatgaaggagttgataactctggcaaaatgccatgagttcttgatatttgaagaccggaagtttgcagatataggaaacacagtgaaaaagcagtatgaaggaggtatctttaaaatagcttcctgggcagatctagtaaatgctcacgtggtgccaggctcaggagttgtgaaaggcctgcaagaagtgggcctgcctttgcatcgggggtgcctccttattgcggaaatgagctccaccggctccctggccactggggactacactagagcagcggttagaatggctgaggagcactctgaatttgttgttggttttatttctggctcccgagtaagcatgaaaccagaatttcttcacttgactccaggagttcagttggaagcaggaggagataatcttggccaacagtacaatagcccacaagaagttattggcaaacgaggttccgatatcatcattgtaggtcgtggcataatctcagcagctgatcgtctggaagcagcagagatgtacagaaaagctgcttgggaagcgtatttgagtagacttggtgttgagggcaggggcagcctgctgacctgcggcgacgtggaggagaaccccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagggcgcgccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctagttgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggctt

SEQ ID NO: 43 - DNA, 4452 bp, Artificial Sequence
  Description of Artificial Sequence Synthetic Construct
ttgtatctataaaaataaacaaaattagcttggtgtggtggcgcctgtagtccccagccacttggaggggtgaggtgagaggattgcttgagcccgggatgatccaggctgcagtgagccatgatcgtgccactgcactccagcctgggcgacagagtgagaccctgtctcacaacaacaacagcaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctctagggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccgccaccatggcggtcgctcgtgcagctttggggccattggtgacgggtctgtacgacgtgcaggctttcaagtttggggacttcgtgctgaagagcgggctttcctcccccatctacatcgatctgcggggcatcgtgtctcgaccgcgtcttctgagtcaggttgcagatattttattccaaactgcccaaaatgcaggcatcagttttgacaccgtgtgtggagtgccttatacagctttgccattggctacagttatctgttcaaccaatcaaattccaatgcttattagaaggaaagaaacaaaggattatggaactaagcgtcttgtagaaggaactattaatccaggagaaacctgtttaatcattgaagatgttgtcaccagtggatctagtgttttggaaactgttgaggttcttcagaaggagggcttgaaggtcactgatgccatagtgctgttggacagagagcagggaggcaaggacaagttgcaggcgcacgggatccgcctccactcagtgtgtacattgtccaaaatgctggagattctcgagcagcagaaaaaagttgatgctgagacagttgggagagtgaagaggtttattcaggagaatgtctttgtggcagcgaatcataatggttctgagggcaggggcagcctgctgacctgcggcgacgtggaggagaaccccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagggcgcgccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctagttgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggctt

SEQ ID NO: 44 - DNA, 4425 bp, Artificial Sequence
  Description of Artificial Sequence Synthetic Construct
cattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctctagggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgcctcgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccgccaccatgcccccccccaggctgctgttcttcctgctgttcctgacccccatggaggtgaggcccgaggagcccctggtggtgaaggtggaggagggcgacaacgccgtgctgcagtgcctgaagggcaccagcgacggccccacccagcagctgacctggagcagggagagccccctgaagcccttcctgaagctgagcctgggcctgcccggcctgggcatccacatgaggcccctggccatctggctgttcatcttcaacgtgagccagcagatgggcggcttctacctgtgccagcccggcccccccagcgagaaggcctggcagcccggctggaccgtgaacgtggagggcagcggcgagctgttcaggtggaacgtgagcgacctgggcggcctgggctgcggcctgaagaacaggagcagcgagggccccagcagccccagcggcaagctgatgagccccaagctgtacgtgtgggccaaggacaggcccgagatctgggagggcgagcccccctgcctgccccccagggacagcctgaaccagagcctgagccaggacctgaccatggcccccggcagcaccctgtggctgagctgcggcgtgccccccgacagcgtgagcaggggccccctgagctggacccacgtgcaccccaagggccccaagagcctgctgagcctggagctgaaggacgacaggcccgccagggacatgtgggtgatggagaccggcctgctgctgcccagggccaccgcccaggacgccggcaagtactactgccacaggggcaacctgaccatgagcttccacctggagatcaccgccaggcccgtgctgtggcactggctgctgaggaccggcggctggaaggtgagcgccgtgaccctggcctacctgatcttctgcctgtgcagcctggtgggcatcctgcacctgcagagggccctggtgctgaggaggaagaggaagaggatgaccgaccccaccaggaggttccgcaagcgccgcagcggcagcggcgagggccgcggcagcctgctgacctgcggcgacgtggaggagaaccccggccccaagcttcccctttctataaaggaagcacccaaagaactcagcttcggtgcacgtgcagagctgcccaggatccacccagttgcatcgaagcttctcaggcttatgcaaaagaaggagaccaatctgtgtctatctgctgatgtttcactggccagagagctgttgcagctagcagatgctttaggacctagtatctgcatgctgaagactcatgtagatattttgaatgattttactctggatgtgatgaaggagttgataactctggcaaaatgccatgagttcttgatatttgaagaccggaagtttgcagatataggaaacacagtgaaaaagcagtatgaaggaggtatctttaaaatagcttcctgggcagatctagtaaatgctcacgtggtgccaggctcaggagttgtgaaaggcctgcaagaagtgggcctgcctttgcatcgggggtgcctccttattgcggaaatgagctccaccggctccctggccactggggactacactagagcagcggttagaatggctgaggagcactctgaatttgttgttggttttatttctggctcccgagtaagcatgaaaccagaatttcttcacttgactccaggagttcagttggaagcaggaggagataatcttggccaacagtacaatagcccacaagaagttattggcaaacgaggttccgatatcatcattgtaggtcgtggcataatctcagcagctgatcgtctggaagcagcagagatgtacagaaaagctgcttgggaagcgtatttgagtagacttggtgtttaactcgagggcgcgccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctagttgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcc